(19) United States
(12) Patent Application Publication
(10) Pub. No.: US 2022/0305049 A1
(43) Pub. Date: Sep. 29, 2022

(54) HEPARIN AND HEPARAN SULFATE FROM MODIFIED MST CELLS AND METHODS OF MAKING AND USING

(71) Applicant: TEGA Therapeutics, Inc., San Diego, CA (US)

(72) Inventors: Charles GLASS, San Diego, CA (US); Bryan THACKER, San Diego, CA (US); Jeffrey D. ESKO, San Diego, CA (US)

(21) Appl. No.: 17/638,760

(22) PCT Filed: Aug. 27, 2020
(86) PCT No.: PCT/US2020/048243

§ 371 Date: Feb. 25, 2022

Related U.S. Application Data
(60) Provisional application No. 63/005,146, filed on Apr. 3, 2020.
(60) Provisional application No. 62/892,477, filed on Aug. 27, 2019.

(51) Int. Cl. A61K 31/727; A61P 29/00; A61P 11/00; A61P 7/02; C12N 15/86; C12N 15/52; C12N 5/09; C12P 19/26

(52) U.S. Cl. CPC A61K 31/727; A61P 29/00; A61P 11/00; A61P 7/02; C12N 15/86; C12N 15/52; C12Y 208/02; C12N 5/0693; C12P 19/26; C12N 2510/02; C12N 2740/15043

(57) ABSTRACT

Abstract: Provided herein are methods of producing heparin and heparan sulfate from modified cells, such as modified MST cells and modified basophil neoplastic cells, and compositions comprising heparin and heparan sulfate isolated from modified cells. In some embodiments, methods herein comprise culturing a genetically modified cell line comprising at least one of a mastocytoma cell line and a basophil neoplastic cell line; and isolating the heparin or heparan sulfate from the cell line.

### CROSS-REFERENCE

This application claims the benefit of U.S. Provisional Application No. 62/892,477, filed Aug. 27, 2019, and U.S. Provisional Application No. 63/005,146, filed Apr. 3, 2020, each of which is incorporated herein by reference in its entirety.

### STATEMENT AS TO FEDERALLY SPONSORED RESEARCH

This invention was made with government support under GM33063 awarded by National Institutes of Health and 1622959 and 1842736 awarded by the National Science Foundation. The government has certain rights in the invention.

### BACKGROUND

Heparin remains an important and widely prescribed drug. Approximately 300,000 doses are administered per day in the United States. An aging population and increased incidence of diseases (e.g. heart failure and diabetes) leads to more procedures like cardiopulmonary bypass surgery where heparin remains the drug of choice. Risks of heparin induced thrombocytopenia (HIT) associated with high doses of heparin administered before and during surgery have led to the introduction of alternative anticoagulant therapies.

### SUMMARY

Provided herein are methods of producing a heparin or a heparan sulfate. In some embodiments, methods herein comprise culturing a genetically modified cell line comprising at least one of a mastocytoma cell line and a basophil neoplastic cell line; and isolating the heparin or heparan sulfate from the cell line. In some embodiments, wherein the mastocytoma cell line is selected from the group consisting of MST cells, P815 cells, MC/9 cells, SI/SI4 cells, 10P2 cells, 11P0-1 cells, and 10P12 cells. In some embodiments, the genetically modified cell line is RT4 cells, 682B cells, 751G cells, 1016T cells, KK-47, MGH-U1, MHG-U2, MGH-U3, or MGH-U4. In some embodiments, the genetically modified cell line is deficient for one or more of chondroitin sulfate synthase 1 (CHSY1), chondroitin sulfate N-acetylgalactosaminyltransferase 2 (CSGALNACT2), chondroitin sulfate N-acetylgalactosaminyltransferase 1 (CSGALNACT1), Heparan sulfate 2-O-sulfotransferase (HS2ST), and heparan sulfate C5-epimerase (GLCE). In some embodiments, the genetically modified cell line is deficient for a heparan sulfate catabolic enzyme. In some embodiments, the heparan sulfate catabolic enzyme comprises one or more of heparanase (HPSE), beta-glucoronidase (GUSB), sulfamidase (SGSH), heparan alpha-glucosaminide N-acetyltransferase (HGNAT), alpha-N-acetyl glucosaminidase (GSNAT), uronate-2-O-sulfatases (IDS and GDS), alpha-L-iduronidase (IDUA), and heparan sulfate 6-O-endosulfatase (SULF1-2). In some embodiments, the genetically modified cell line overexpresses one or more of heparanase, a protease, a polymerase, a sulfotransferase, an endosulfatase, and a proteoglycan protein core. In some embodiments, the genetically modified cell line overexpresses one or more sulfotransferases. In some embodiments, the protease comprises one or more of matrix metalloproteinase (MMP), MMP2, MMP3, MMPI, MMP9, MT1-MMP, MT3-MMP, ADAM17, ADAMTS1, ADAMTS4, trypsin, and chymotrypsin. In some embodiments, the proteoglycan protein core comprises one or more of a serglycin, a syndecan, a glypican, perlecan, and CD44 core protein (CD44E). In some embodiments, the serglycin comprises a human serglycin, a pig serglycin, a mouse serglycin. In some embodiments, the number of GAG attachment sites in the serglycin is modified. In some embodiments, the syndecan is selected from the group consisting of syndecan-1, syndecan-2, syndecan-3, syndecan-4, and syndecan ectodomains. In some embodiments, the glypican is selected from the group consisting of glypican 1, glypican 2, glypican 3, glypican 4, glypican 5, and glypican 6 and ectodomains. In some embodiments, the genetically modified cell line overexpresses one or more of Hs3st1, Hs6st1, Hs6st2, Ndst2, or Sulf2. In some embodiments, the genetically modified cell line overexpresses Hs3st1. In some embodiments, the genetically modified cell line overexpresses Hs3st1 and Hs6st1. In some embodiments, the genetically modified cell line overexpresses Hs3st1, Hs6st1, and Sulf2. In some embodiments, the method comprises degranulating the cell line. In some embodiments, degranulating the cell line comprises a method selected from the group consisting of Antigen-IgE induced FcεRI aggregation on the degranulating cell surface, contacting the cell line to a degranulating agent, altering the culture temperature, altering the culture medium pH, altering the culture medium salt concentration, and agitation. In some embodiments, the degranulating agent is selected from the group consisting of calcium ionophore A23187, compound 48/80, tetradecanoyl phorbol acetate (TPA), and substance P. In some embodiments, the genetically modified cell line is cultured in CDM4NS0 medium. In some embodiments, the genetically modified cell line is cultured in a medium supplemented with xylosides. In some embodiments, the genetically modified cell line is cultured in suspension culture or in a hollow fiber bioreactor.

Also provided herein are high throughput methods of quantifying heparan sulfate in a group of samples. In some embodiments, the methods comprise: (a) binding each sample to a well of a multi-well chromatography column; (b) digesting the samples bound to the column with an enzyme; (c) eluting the samples from the column with a solution comprising a salt; and (d) measuring the heparan sulfate in the sample using liquid chromatography. In some embodiments, the chromatography column is selected from at least one of an ion exchange column and a size exclusion column. In some embodiments, the enzyme is selected from at least one of a nuclease and a protease. In some embodiments, the salt is a volatile salt. In some embodiments, the liquid chromatography is an ultra performance liquid chromatography. In some embodiments, the method comprises liquid chromatography with fluorescently tagged heparan sulfate disaccharides.

Also provided herein are high throughput methods of quantifying heparan sulfate in a group of samples, the methods comprising: (a) contacting each sample to a well in a multi-well plate, wherein each well is coated with a guanidinylated antibiotic, thereby binding the heparan sulfate in the sample to the plate; (b) contacting the bound heparan sulfate to a heparan sulfate binding protein; (c) contacting the bound heparan sulfate binding protein to a detection reagent; and (d) measuring a signal from the detection reagent, wherein the signal from the detection reagent corresponds to the amount of heparan sulfate in the sample. In some embodiments, the guanidinylated antibiotic comprises guanidinylated neomycin. In some embodiments, the heparan sulfate binding protein is selected from at least one of FGF-2, PF4, ATIII, and VEGF. In some embodiments, the signal is selected from at least one of a fluorescent signal; a luminescent signal; and a colorimetric signal. In some embodiments, the signal is generated enzymatically.

Further provided herein are methods of culturing a stem cell, the methods comprising contacting a stem cell to a composition comprising a heparin or a heparan sulfate isolated from a genetically modified cell line. In some embodiments, the genetically modified cell line comprises at least one of a mastocytoma (MST) cell line and a basophil neoplastic cell line. In some embodiments, the genetically modified cell line is deficient for one or more of chondroitin sulfate synthase 1 (CHSY1), chondroitin sulfate N-acetylgalactosaminyltransferase 2 (CSGALNACT2), chondroitin sulfate N-acetylgalactosaminyltransferase 1 (CSGALNACT1), Heparan sulfate 2-O-sulfotransferase (HS2ST), and heparan sulfate C5-epimerase (GLCE). In some embodiments, the genetically modified cell line is deficient for a heparan sulfate catabolic enzyme. In some embodiments, the heparan sulfate catabolic enzyme comprises one or more of heparanase (HPSE), beta-glucoronidase (GUSB), sulfamidase (SGSH), heparan alpha-glucosaminide N-acetyltransferase (HGNAT), alpha-N-acetyl glucosaminidase (GSNAT), uronate-2-O-sulfatases (IDS), alpha-L-iduronidase (IDUA), and heparan sulfate 6-O-endosulfatase (SULF1-2). In some embodiments, the genetically modified cell line overexpresses one or more of heparanase, a protease, a polymerase, a sulfotransferase, an endosulfatase, and a proteoglycan protein core. In some embodiments, the protease comprises one or more of matrix metalloproteinase (MMP), MMP2, MMP3, MMP7, MMP9, MT1-MMP, MT3-MMP, ADAM17, ADAMTS1, ADAMTS4, trypsin, and chymotrypsin. In some embodiments, the proteoglycan protein core comprises one or more of a serglycin, a syndecan, a glypican, perlecan, and CD44 core protein (CD44E). In some embodiments, the serglycin comprises a human serglycin, a pig serglycin, a mouse serglycin. In some embodiments, the number of GAG attachment sites in the serglycin is modified. In some embodiments, the syndecan is selected from the group consisting of syndecan-1, syndecan-2, syndecan-3, syndecan-4, and syndecan ectodomains. In some embodiments, the glypican is selected from the group consisting of glypican 1, glypican 2, glypican 3, glypican 4, glypican 5, and glypican 6 and ectodomains.

Also provided herein are methods of treating a wound in a subject. In some embodiments, the methods comprise contacting the wound to a composition comprising a heparin or a heparan sulfate isolated from a genetically modified cell line. In some embodiments, the genetically modified cell line comprises at least one of a mastocytoma (MST) cell line and a basophil neoplastic cell line. In some embodiments, the wound is selected from at least one of a diabetic wound, an incision, a laceration, an abrasion, an avulsion, a puncture wound, a penetration wound, a gunshot wound, a hematoma, and a crush injury. In some embodiments, the genetically modified cell line is deficient for one or more of chondroitin sulfate synthase 1 (CHSY1), chondroitin sulfate N-acetylgalactosaminyltransferase 2 (CSGALNACT2), chondroitin sulfate N-acetylgalactosaminyltransferase 1 (CSGALNACT1), Heparan sulfate 2-O-sulfotransferase (HS2ST), and heparan sulfate C5-epimerase (GLCE). In some embodiments, the genetically modified cell line is deficient for a heparan sulfate catabolic enzyme. In some embodiments, the heparan sulfate catabolic enzyme comprises one or more of heparanase (HPSE), beta-glucoronidase (GUSB), sulfamidase (SGSH), heparan alpha-glucosaminide N-acetyltransferase (HGNAT), alpha-N-acetyl glucosaminidase (GSNAT), uronate-2-O-sulfatase (IDS and GDS), alpha-L-iduronidase (IDUA), and heparan sulfate 6-O-endosulfatase (SULF1-2). In some embodiments, the genetically modified MST cell line overexpresses one or more of heparanase, a protease, a polymerase, a sulfotransferase, an endosulfatase, and a proteoglycan protein core. In some embodiments, the protease comprises one or more of matrix metalloproteinase (MMP), MMP2, MMP3, MMP7, MMP9, MT1-MMP, MT3-MMP, ADAM17, ADAMTS1, ADAMTS4, trypsin, and chymotrypsin. In some embodiments, the proteoglycan protein core comprises one or more of a serglycin, a syndecan, a glypican, perlecan, and CD44 core protein (CD44E). In some embodiments, the serglycin comprises a human serglycin, a pig serglycin, a mouse serglycin. In some embodiments, the number of GAG attachment sites in the serglycin is modified. In some embodiments, the syndecan is selected from the group consisting of syndecan-1, syndecan-2, syndecan-3, syndecan-4, and syndecan ectodomains. In some embodiments, the glypican is selected from the group consisting of glypican 1, glypican 2, glypican 3, glypican 4, glypican 5, and glypican 6 and ectodomains.

Further provided herein are methods of treating a bone fracture in a subject. In some embodiments, the methods comprise administering an effective amount of a composition comprising a heparin or a heparan sulfate isolated from a genetically modified cell line. In some embodiments, the genetically modified cell line comprises at least one of a mastocytoma (MST) cell line and a basophil neoplastic cell line. In some embodiments, the genetically modified cell line is deficient for one or more of chondroitin sulfate synthase 1 (CHSY1), chondroitin sulfate N-acetylgalactosaminyltransferase 2 (CSGALNACT2), chondroitin sulfate N-acetylgalactosaminyltransferase 1 (CSGALNACT1), Heparan sulfate 2-O-sulfotransferase (HS2ST), and heparan sulfate C5-epimerase (GLCE). In some embodiments, the genetically modified cell line is deficient for a heparan sulfate catabolic enzyme. In some embodiments, the heparan sulfate catabolic enzyme comprises one or more of heparanase (HPSE), beta-glucoronidase (GUSB), sulfamidase (SGSH), heparan alpha-glucosaminide N-acetyltransferase (HGNAT), alpha-N-acetyl glucosaminidase (GSNAT), uronate-2-O-sulfatase (IDS and GDS), alpha-L-iduronidase (IDUA), and heparan sulfate 6-O-endosulfatase (SULF1-2). In some embodiments, the genetically modified cell line overexpresses one or more of heparanase, a protease, a polymerase, a sulfotransferase, an endosulfatase, and a proteoglycan protein core. In some embodiments, the protease comprises one or more of matrix metalloproteinase (MMP), MMP2, MMP3, MMP7, MMP9, MT1-MMP, MT3-MMP, ADAM17, ADAMTS1, ADAMTS4, trypsin, and chymotrypsin. In some embodiments, the proteoglycan protein core comprises one or more of a serglycin, a syndecan, a glypican, perlecan, and CD44 core protein (CD44E). In some embodiments, the serglycin comprises a human serglycin, a pig serglycin, a mouse serglycin. In some embodiments, the number of GAG attachment sites in the serglycin is modified. In some embodiments, the syndecan is selected from the group consisting of syndecan-1, syndecan-2, syndecan-3, syndecan-4, and syndecan ectodomains. In some embodiments, the glypican is selected from the group consisting of glypican 1, glypican 2, glypican 3, glypican 4, glypican 5, and glypican 6 and ectodomains.

Also provided herein are methods of reducing the appearance of a skin condition. In some embodiments, the methods comprise contacting the skin condition to a composition comprising a heparin or a heparan sulfate isolated from a genetically modified cell line. In some embodiments, the genetically modified cell line comprises at least one of a mastocytoma (MST) cell line and a basophil neoplastic cell line. In some embodiments, the skin condition is at least one of a wrinkle, a pimple, a hyperpigmentation, an age-related skin condition, dryness, lack of skin elasticity, lack of skin firmness, and fine lines. In some embodiments, the genetically modified cell line is deficient for one or more of chondroitin sulfate synthase 1 (CHSY1), chondroitin sulfate N-acetylgalactosaminyltransferase 2 (CSGALNACT2), chondroitin sulfate N-acetylgalactosaminyltransferase 1 (CSGALNACT1), Heparan sulfate 2-O-sulfotransferase (HS2ST), and heparan sulfate C5-epimerase (GLCE). In some embodiments, the genetically modified cell line is deficient for a heparan sulfate catabolic enzyme. In some embodiments, the heparan sulfate catabolic enzyme comprises one or more of heparanase (HPSE), beta-glucoronidase (GUSB), sulfamidase (SGSH), heparan alpha-glucosaminide N-acetyltransferase (HGNAT), alpha-N-acetyl glucosaminidase (GSNAT), uronate-2-O-sulfatases (IDS and GDS), alpha-L-iduronidase (IDUA), and heparan sulfate 6-O-endosulfatase (SULF1-2). In some embodiments, the genetically modified cell line overexpresses one or more of heparanase, a protease, a polymerase, a sulfotransferase, an endosulfatase, and a proteoglycan protein core. In some embodiments, the protease comprises one or more of matrix metalloproteinase (MMP), MMP2, MMP3, MMP7, MMP9, MT1-MMP, MT3-MMP, ADAM17, ADAMTS1, ADAMTS4, trypsin, and chymotrypsin. In some embodiments, the proteoglycan protein core comprises one or more of a serglycin, a syndecan, a glypican, perlecan, and CD44 core protein (CD44E). In some embodiments, the serglycin comprises a human serglycin, a pig serglycin, a mouse serglycin. In some embodiments, the number of GAG attachment sites in the serglycin is modified. In some embodiments, the syndecan is selected from the group consisting of syndecan-1, syndecan-2, syndecan-3, syndecan-4, and syndecan ectodomains. In some embodiments, the glypican is selected from the group consisting of glypican 1, glypican 2, glypican 3, glypican 4, glypican 5, and glypican 6 and ectodomains.

Further provided herein are methods of treating a cancer in a subject. In some embodiments, the methods comprise to the subject an effective amount of a composition comprising a heparin or a heparan sulfate isolated from a genetically modified cell line. In some embodiments, the genetically modified cell line comprises at least one of a mastocytoma (MST) cell line and a basophil neoplastic cell line. In some embodiments, the cancer is selected from the group consisting of a skin cancer, a lung cancer, a breast cancer, a prostate cancer, a colorectal cancer, a bladder cancer, a melanoma, a lymphoma, a kidney cancer, and a leukemia. In some embodiments, the genetically modified cell line is deficient for one or more of chondroitin sulfate synthase 1 (CHSY1), chondroitin sulfate N-acetylgalactosaminyltransferase 2 (CSGALNACT2), chondroitin sulfate N-acetylgalactosaminyltransferase 1 (CSGALNACT1), Heparan sulfate 2-O-sulfotransferase (HS2ST), and heparan sulfate C5-epimerase (GLCE). In some embodiments, the genetically modified cell line is deficient for a heparan sulfate catabolic enzyme. In some embodiments, the heparan sulfate catabolic enzyme comprises one or more of heparanase (HPSE), beta-glucoronidase (GUSB), sulfamidase (SGSH), heparan alpha-glucosaminide N-acetyltransferase (HGNAT), alpha-N-acetyl glucosaminidase (GSNAT), uronate-2-O-sulfatases (IDS and GDS), alpha-L-iduronidase (IDUA), and heparan sulfate 6-O-endosulfatase (SULF1-2). In some embodiments, the genetically modified cell line overexpresses one or more of heparanase, a protease, a polymerase, a sulfotransferase, an endosulfatase, and a proteoglycan protein core. In some embodiments, the protease comprises one or more of matrix metalloproteinase (MMP), MMP2, MMP3, MMP7, MMP9, MT1-MMP, MT3-MMP, ADAM17, ADAMTS1, ADAMTS4, trypsin, and chymotrypsin. In some embodiments, the proteoglycan protein core comprises one or more of a serglycin, a syndecan, a glypican, perlecan, and CD44 core protein (CD44E). In some embodiments, the serglycin comprises a human serglycin, a pig serglycin, a mouse serglycin. In some embodiments, the number of GAG attachment sites in the serglycin is modified. In some embodiments, the syndecan is selected from the group consisting of syndecan-1, syndecan-2, syndecan-3, syndecan-4, and syndecan ectodomains. In some embodiments, the glypican is selected from the group consisting of glypican 1, glypican 2, glypican 3, glypican 4, glypican 5, and glypican 6 and ectodomains.

Also provided herein are methods of modulating angiogenesis in a subject. In some embodiments, the methods comprise to the subject an effective amount of a composition comprising a heparin or a heparan sulfate isolated from a genetically modified cell line. In some embodiments, the genetically modified cell line comprises at least one of a mastocytoma (MST) cell line and a basophil neoplastic cell line. In some embodiments, the method increases angiogenesis in the subject. In some embodiments, the method decreases angiogenesis in the subject. In some embodiments, the genetically modified cell line is deficient for one or more of chondroitin sulfate synthase 1 (CHSY1), chondroitin sulfate N-acetylgalactosaminyltransferase 2 (CSGALNACT2), chondroitin sulfate N-acetylgalactosaminyltransferase 1 (CSGALNACT1), Heparan sulfate 2-O-sulfotransferase (HS2ST), and heparan sulfate C5-epimerase (GLCE). In some embodiments, the genetically modified cell line is deficient for a heparan sulfate catabolic enzyme. In some embodiments, the heparan sulfate catabolic enzyme comprises one or more of heparanase (HPSE), beta-glucoronidase (GUSB), sulfamidase (SGSH), heparan alpha-glucosaminide N-acetyltransferase (HGNAT), alpha-N-acetyl glucosaminidase (GSNAT), uronate-2-O-sulfatases (IDS and GDS), alpha-L-iduronidase (IDUA), and heparan sulfate 6-O-endosulfatase (SULF1-2). In some embodiments, the genetically modified cell line overexpresses one or more of heparanase, a protease, a polymerase, a sulfotransferase, an endosulfatase, and a proteoglycan protein core. In some embodiments, the protease comprises one or more of matrix metalloproteinase (MMP), MMP2, MMP3, MMP7, MMP9, MT1-MMP, MT3-MMP, ADAM17, ADAMTS1, ADAMTS4, trypsin, and chymotrypsin. In some embodiments, the proteoglycan protein core comprises one or more of a serglycin, a syndecan, a glypican, perlecan, and CD44 core protein (CD44E). In some embodiments, the serglycin comprises a human serglycin, a pig serglycin, a mouse serglycin. In some embodiments, the number of GAG attachment sites in the serglycin is modified. In some embodiments, the syndecan is selected from the group consisting of syndecan-1, syndecan-2, syndecan-3, syndecan-4, and syndecan ectodomains. In some embodiments, the glypican is selected from the group consisting of glypican 1, glypican 2, glypican 3, glypican 4, glypican 5, and glypican 6 and ectodomains.

Also provided herein are methods of treating an inflammatory disease in a subject. In some embodiments, the methods comprise to the subject an effective amount of a composition comprising a heparin or a heparan sulfate isolated from a genetically modified cell line. In some embodiments, the genetically modified cell line comprises at least one of a mastocytoma (MST) cell line and a basophil neoplastic cell line. In some embodiments, the inflammatory disease is selected from at least one of the group consisting of chronic obstructive pulmonary disease (COPD), acute respiratory distress syndrome (ARDS), cystic fibrosis, alpha-1 antitrypsin deficiency, a diabetes, an arthritis, psoriasis, multiple sclerosis, systemic lupus erythematosus, an inflammatory bowel disease, Graves' disease, Addison's disease, Sjogren's syndrome, Hashimoto's thyroiditis, Myasthenia gravis, vasculitis, an anemia, and celiac disease. In some embodiments, the genetically modified cell line is deficient for one or more of chondroitin sulfate synthase 1 (CHSY1), chondroitin sulfate N-acetylgalactosaminyltransferase 2 (CSGALNACT2), chondroitin sulfate N-acetylgalactosaminyltransferase 1 (CSGALNACT1), Heparan sulfate 2-O-sulfotransferase (HS2ST), and heparan sulfate C5-epimerase (GLCE). In some embodiments, the genetically modified cell line is deficient for a heparan sulfate catabolic enzyme.

Also included herein are methods of treating wherein the heparan sulfate catabolic enzyme comprises one or more of heparanase (HPSE), beta-glucoronidase (GUSB), sulfamidase (SGSH), heparan alpha-glucosaminide N-acetyltransferase (HGNAT), alpha-N-acetyl glucosaminidase (GSNAT), uronate-2-O-sulfatases (IDS and GDS), alpha-L-iduronidase (IDUA), and heparan sulfate 6-O-endosulfatase (SULF1-2). In some embodiments, the genetically modified cell line overexpresses one or more of heparanase, a protease, a polymerase, a sulfotransferase, an endosulfatase, and a proteoglycan protein core. In some embodiments, the protease comprises one or more of matrix metalloproteinase (MMP), MMP2, MMP3, MMP7, MMP9, MT1-MMP, MT3-MMP, ADAM17, ADAMTS1, ADAMTS4, trypsin, and chymotrypsin. In some embodiments, the proteoglycan protein core comprises one or more of a serglycin, a syndecan, a glypican, perlecan, and CD44 core protein (CD44E). In some embodiments, the serglycin comprises a human serglycin, a pig serglycin, a mouse serglycin. In some embodiments, the number of GAG attachment sites in the serglycin is modified. In some embodiments, the syndecan is selected from the group consisting of syndecan-1, syndecan-2, syndecan-3, syndecan-4, and syndecan ectodomains. In some embodiments, the glypican is selected from the group consisting of glypican 1, glypican 2, glypican 3, glypican 4, glypican 5, and glypican 6 and ectodomains.

Also provided herein are methods of treating a neurological disease or condition in a subject. In some embodiments, the methods comprise to the subject an effective amount of a composition comprising a heparin or a heparan sulfate isolated from a genetically modified cell line. In some embodiments, the genetically modified cell line comprises at least one of a mastocytoma (MST) cell line and a basophil neoplastic cell line. In some embodiments, the neurological disease or condition is selected from at least one of the group consisting of Alzheimer's disease, Parkinson's disease, Huntington's disease, Amyotrophic lateral sclerosis, Dementia, Transmissible spongiform encephalopathy, Dentatorubro-pallidoluysian atrophy, Spinal and bulbar muscular atrophy, Spinocerebellar ataxia Type 1, Spinocerebellar ataxia Type 2, Spinocerebellar ataxia Type 3, Spinocerebellar ataxia Type 6, Spinocerebellar ataxia Type 7, and Spinocerebellar ataxia Type 17. In some embodiments, the genetically modified cell line is deficient for one or more of chondroitin sulfate synthase 1 (CHSY1), chondroitin sulfate N-acetylgalactosaminyltransferase 2 (CSGALNACT2), chondroitin sulfate N-acetylgalactosaminyltransferase 1 (CSGALNACT1), Heparan sulfate 2-O-sulfotransferase (HS2ST), and heparan sulfate C5-epimerase (GLCE). In some embodiments, the genetically modified cell line is deficient for a heparan sulfate catabolic enzyme. In some embodiments, the heparan sulfate catabolic enzyme comprises one or more of heparanase (HPSE), beta-glucoronidase (GUSB), sulfamidase (SGSH), heparan alpha-glucosaminide N-acetyltransferase (HGNAT), alpha-N-acetyl glucosaminidase (GSNAT), uronate-2-O-sulfatases (IDS and GDS), alpha-L-iduronidase (IDUA), and heparan sulfate 6-O-endosulfatase (SULF1-2). In some embodiments, the genetically modified cell line overexpresses one or more of heparanase, a protease, a polymerase, a sulfotransferase, an endosulfatase, and a proteoglycan protein core. In some embodiments, the protease comprises one or more of matrix metalloproteinase (MMP), MMP2, MMP3, MMP7, MMP9, MT1-MMP, MT3-MMP, ADAM17, ADAMTS1, ADAMTS4, trypsin, and chymotrypsin. In some embodiments, the proteoglycan protein core comprises one or more of a serglycin, a syndecan, a glypican, perlecan, and CD44 core protein (CD44E). In some embodiments, the serglycin comprises a human serglycin, a pig serglycin, a mouse serglycin. In some embodiments, the number of GAG attachment sites in the serglycin is modified. In some embodiments, the syndecan is selected from the group consisting of syndecan-1, syndecan-2, syndecan-3, syndecan-4, and syndecan ectodomains. In some embodiments, the glypican is selected from the group consisting of glypican 1, glypican 2, glypican 3, glypican 4, glypican 5, and glypican 6 and ectodomains.

Also provided herein are compositions comprising a heparin or a heparan sulfate, wherein the heparin or heparan sulfate has reduced 2-O-sulfation and wherein the heparin or heparan sulfate has reduced affinity for platelet factor 4 (PF4) than an unfractionated heparin preparation. In some embodiments, the composition is purified from a mastocytoma (MST) cell line or a basophil neoplastic cell line genetically modified to be deficient for Heparan sulfate 2-O-sulfotransferase (HS2ST). In some embodiments, the composition is purified from a MST cell line or a basophil neoplastic cell line genetically modified to overexpress Heparan sulfate-6-O-endosulfatase 2 (Sulf-2).

Further provided herein are methods of reducing blood clots in a subject. In some embodiments, the methods comprise administering a composition comprising a heparin or a heparan sulfate having reduced affinity for platelet factor 4 compared to an unfractionated heparin preparation. In some embodiments, the composition is purified from a mastocytoma (MST) cell line or a basophil neoplastic cell line genetically modified to be deficient for Heparan sulfate 2-O-sulfotransferase (HS2ST). In some embodiments, the composition is purified from a MST cell line or a basophil neoplastic cell line genetically modified to overexpress Heparan sulfate-6-O-endosulfatase 2 (Sulf-2). In some embodiments, the composition is purified from a MST cell line or a basophil neoplastic cell line genetically modified to be deficient for one or more of chondroitin sulfate synthase 1 (CHSY1), chondroitin sulfate N-acetylgalactosaminyltransferase 2 (CSGALNACT2), chondroitin sulfate N-acetylgalactosaminyltransferase 1 (CSGALNACT1), Heparan sulfate 2-O-sulfotransferase (HS2ST), and heparan sulfate C5-epimerase (GLCE). In some embodiments, the composition is purified from a MST cell line or a basophil neoplastic cell line genetically modified to be deficient for a heparan sulfate catabolic enzyme. In some embodiments, the heparan sulfate catabolic enzyme comprises one or more of heparanase (HPSE), beta-glucoronidase (GUSB), sulfamidase (SGSH), heparan alpha-glucosaminide N-acetyltransferase (HGNAT), alpha-N-acetyl glucosaminidase (GSNAT), uronate-2-O-sulfatases (IDS and GDS), alpha-L-iduronidase (IDUA), and heparan sulfate 6-O-endosulfatase (SULF1-2). In some embodiments, the composition is purified from a MST cell line or a basophil neoplastic cell line genetically modified to overexpress one or more of heparanase, a protease, a polymerase, a sulfotransferase, an endosulfatase, and a proteoglycan protein core. In some embodiments, the protease comprises one or more of matrix metalloproteinase (MMP), MMP2, MMP3, MMP7, MMP9, MT1-MMP, MT3-MMP, ADAM17, ADAMTS1, ADAMTS4, trypsin, and chymotrypsin. In some embodiments, the proteoglycan protein core comprises one or more of a serglycin, a syndecan, a glypican, perlecan, and CD44 core protein (CD44E). In some embodiments, the serglycin comprises a human serglycin, a pig serglycin, a mouse serglycin. In some embodiments, the number of GAG attachment sites in the serglycin is modified. In some embodiments, the syndecan is selected from the group consisting of syndecan-1, syndecan-2, syndecan-3, syndecan-4, and syndecan ectodomains. In some embodiments, the glypican is selected from the group consisting of glypican 1, glypican 2, glypican 3, glypican 4, glypican 5, and glypican 6 and ectodomains.

Further provided herein are compositions comprising a heparin or a heparan sulfate. In some embodiments, the heparin or heparan sulfate has increased 2-O-sulfate compared to pharmaceutical heparin. In some embodiments, the heparin or the heparan sulfate has improved anti-inflammatory activity compared with pharmaceutical heparin. In some embodiments, the composition is purified from a mastocytoma (MST) cell line or a basophil neoplastic cell line genetically modified to be overexpress Heparan sulfate 2-O-sulfotransferase (HS2ST).

Additionally provided herein are methods of treating a lung inflammatory disease in a subject. In some embodiments, the method comprising to the subject an effective amount of a composition comprising a heparin or a heparan sulfate isolated from a genetically modified cell line. In some embodiments, the genetically modified cell line comprises at least one of a mastocytoma (MST) cell line and a basophil neoplastic cell line. In some embodiments, the inflammatory disease is selected from at least one of the group consisting of chronic obstructive pulmonary disease (COPD), acute respiratory distress syndrome (ARDS), cystic fibrosis, alpha-1 antitrypsin deficiency, and an asthma. In some embodiments, the genetically modified cell line is deficient for one or more of chondroitin sulfate synthase 1 (CHSY1), chondroitin sulfate N-acetylgalactosaminyltransferase 2 (CSGALNACT2), chondroitin sulfate N-acetylgalactosaminyltransferase 1 (CSGALNACT1), and heparan sulfate C5-epimerase (GLCE). In some embodiments, the genetically modified cell line is deficient for a heparan sulfate catabolic enzyme. In some embodiments, the heparan sulfate catabolic enzyme comprises one or more of heparanase (HPSE), beta-glucoronidase (GUSB), sulfamidase (SGSH), heparan alpha-glucosaminide N-acetyltransferase (HGNAT), alpha-N-acetyl glucosaminidase (GSNAT), uronate-2-O-sulfatases (IDS and GDS), alpha-L-iduronidase (IDUA), and heparan sulfate 6-O-endosulfatase (SULF1-2). In some embodiments, the genetically modified cell line overexpresses one or more of heparanase, a protease, a polymerase, a sulfotransferase, an endosulfatase, and a proteoglycan protein core. In some embodiments, the sulfotransferase is Heparan Sulfate 2-O-Sulfotransferase 1.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

An understanding of the features and advantages of the present invention will be obtained by reference to the following detailed description that sets forth illustrative embodiments, in which the principles of the invention are utilized, and the accompanying drawings of which:

FIG. 1 illustrates an AT3 binding pentasaccharide.

FIG. 2 illustrates MST viable cell density results in 1 L flasks.

FIG. 3 illustrates MST viable cell density results in 125 ml flasks.

FIG. 4 illustrates GAG yield from MST cells cultured in 1 L shaker flasks.

FIG. 5 illustrates GAG yield from MST cells cultured in 125 ml shaker flasks.

FIG. 6 illustrates MST GAG yield.

FIG. 7 illustrates MST HS disaccharide content.

FIG. 8 illustrates Factor Xa inactivation results.

FIG. 9 illustrates the disaccharide composition of HS from RT4 cells as determined by UPLC. The types of disaccharides are shown on the x-axis using the disaccharide structural code.

FIGS. 10A-10D illustrate LCMS analysis of CS knockout cell lines. FIG. 10A and FIG. 10B show WT MST cells and FIG. 10C and FIG. 10D show MST17B10. LCMS analysis detects HS and CS in the purified material.

FIG. 11 illustrates dissacharide composition and FXa analysis of transduced cell populations.

FIGS. 12A-12F illustrate characterization of MST38 rHS. FIG. 12A shows anti-FXa activity. FIG. 12B shows anti-FIla activity. FIG. 12C shows protamine neutralization of the FXa assay. FIG. 12D shows the sulfate content of MST38 rHS compared with pharmaceutical heparin. FIG. 12E shows results of administration of PBS, MST38 rHS, or pharmaceutical heparin to C57B16 mice. FIG. 12F shows plate binding of heparin and MST38 rHS to biotin-PF4.

FIGS. 13A-13C shows characterization of rHS from cells overexpressing Sulf2. FIG. 13A shows HS yield. FIG. 13B shows PF4 binding. FIG. 13C shows anti-FXa activity.

FIGS. 14A-14D show test rHS production in serum free media. FIG. 14A and FIG. 14B show cell growth of ChA27 and MST17B10 in various media. FIG. 14C shows HS yield of cells in various media. FIG. 14D shows HS sulfate content of cells in various media.

FIGS. 15A-15C show IVCD (FIG. 15A), rHS yield (FIG. 15B), and anti-FXa activity (FIG. 15C) of cells grown in serum free medium.

FIGS. 16A-16B shows HS yield of ChA27 cells (FIG. 16A) and MST cells (FIG. 16B) grown in culture medium supplemented with xylosides.

FIGS. 17A-17B show measurement of intracellular rHS by flow cytometry measuring rHS bound to ATIII (FIG. 17A) or FGF2 (FIG. 17B).

FIG. 18 shows rHS sulfate content was determined for MST cells grown in medium supplemented with 1 mM Na2SO4.

FIG. 19 shows inhibition of SARS-CoV-2 binding.

FIG. 20 shows a molecular model of COPD. Heparin is a known inhibitor of factors in red.

FIG. 21 shows 3-O-sulfate (red) containing pentasaccharide responsible for the anticoagulant activity of heparin.

FIG. 22 shows sulfate content of selected non-anticoagulant rHSs and commercially available heparin derivatives. Disaccharide composition for rHS or commercial heparins was determined by exhaustive depolymerization of the polysaccharide followed by UPLC analysis. Compositions identified by rHS number. H: heparin. ODSH: 2-O-, 3-O-desulfated heparin. Each bar from left to right is no sulfate, N-sulfate, 2-O-sulfate, or 6-O-sulfate for each drug candidate tested.

FIG. 23 shows inhibition and competition assays for selected inflammatory targets. Pharmaceutical heparin (UFH), ODSH, rHS09 and rHS01 were tested for their ability to inhibit 80 nM neutrophil elastase (top panel) and compete 10 nM IL8 (middle panel) or CXCL1 (bottom panel) away from 50 ng immobilized heparin. Curves were fit to the data and IC50 values were calculated. Experiments were performed twice with duplicate wells for each experiment. A representative experiment is shown.

### DETAILED DESCRIPTION

Recombinant heparin provides an opportunity to reduce the risk of HIT by engineering the molecular structure of cell-produced heparin. Provided herein are engineered cells and cell lines derived from mastocytoma cells for commercial production of heparin. In vivo, heparin is produced exclusively in mast cells and stored in cytoplasmic granules with histamine and a large number of other inflammatory mediators. Pharmaceutical heparin consists of highly processed material purified from animal tissues. Its anticoagulant properties have made pharmaceutical heparin an extremely important and widely prescribed drug. Heparin is used routinely for the treatment and prophylaxis of thrombosis. Currently, pharmaceutical heparin is purified from porcine intestinal mucosa, sourced predominately from pig slaughterhouses. Such sourcing has led to quality control issues in the heparin supply. Pig blue ear disease significantly reduced the pig population resulting in a shortage of crude heparin and adulteration of the supply resulted in the deaths of over 80 people in the United States and many more worldwide. These occurrences highlight the difficulty in monitoring and controlling the heparin supply chain. A source of bioengineered heparin that is produced using mammalian cell culture under GMP conditions would largely de-risk the heparin supply chain.

In addition to a safer supply chain, producing heparin in cell culture provides an opportunity to genetically engineer improved properties into the molecule, influencing pharmacokinetics, potency and side effects. For example, heparin induced thrombocytopenia (HIT) is a side effect experienced by up to 3 percent of patients treated with heparin. These patients experience an increased risk of thrombosis and thrombocytopenia. Research has shown that the anticoagulant activity in heparin can be separated from the HIT activity, which is feasible by genetically engineering the cell line used to produce the heparin (Pempe, E. H., Burch, T. C., Law, C. J., and Liu, J. (2012) Substrate specificity of 6-O-endosulfatase (Sulf-2) and its implications in synthesizing anticoagulant heparan sulfate. Glycobiology 22, 1353-1362; Stringer, S. E., and Gallagher, J. T. (1997) Specific binding of the chemokine platelet factor 4 to heparan sulfate. J Biol Chem 272, 20508-20514).

Heparin consists of long polysaccharide chains made up of repeating disaccharides, highly modified by epimerization and addition of sulfate groups to certain positions of the sugar residues. The configuration and density of the sulfate groups gives heparin its potent anticoagulant activity. Heparin and heparan sulfate are produced by a biosynthetic pathway comprising over twenty enzymes that are responsible for polymerization, epimerization, sulfation, and phosphorylation. While, enzymes in the pathway are known and have been cloned cell based production of heparin and heparan sulfate remains difficult. Further, model systems are used for heparin research because isolation, propagation, and maintenance of primary connective tissue mast cells is extremely challenging. The Furth murine mastocytoma was an early model used to study heparin biosynthesis. Stable clonal mastocytoma cell lines (MST cells) that produce heparin and chondroitin sulfate chains on the core protein serglycin in cytoplasmic granules were isolated from a tumor cell suspension. Heparin chains produced by the MST cells have structures similar to pharmaceutical heparin except for reduced levels of the 3-O-sulfate modification required for anticoagulant activity. To assess whether this deficiency could be overcome, the MST cells were stably transfected with the heparan sulfate 3-O-sulfotransferase-1 (Hs3st1) enzyme sequence. The transfected cell line (MST-10H) produces the heparin structures required for anticoagulant activity and greatly increased anticoagulant activity (Factor Xa inhibition) (Gasimli, L., Glass, C. A., Datta, P., Yang, B., Li, G., Gemmill, T. R., Baik, J. Y., Sharfstein, S. T., Esko, J. D., and Linhardt, R. J. (2014) Bioengineering murine mastocytoma cells to produce anticoagulant heparin. Glycobiology 24, 272-280). Provided herein are cell culture conditions and processing methods that increase production of heparin by cell lines. Also provided herein are genetic engineering methods that increase potency and production of heparin in cell culture that also reduce the risk of HIT.

### Methods of Producing Heparin and Heparan Sulfate

Provided herein are methods of producing heparin and heparan sulfate in cultured cells and isolating the heparin or heparan sulfate from the cells. Exemplary cultured cells for such methods include mastocytoma cell lines, such as MST cells, P815 cells, MC/9 cells SI/SI4 cells, 10P2 cells, 11P0-1 cells and 10P12 cells; RT4 cells; 682B cells; 751G cells; 1016T cells; KK-47; MGH-U1; MHG-U2; MGH-U3; MGH-U4; and basophil neoplastic cell lines. Specifically contemplated herein are genetically modified cell lines comprising gene deletions and/or gene overexpression to optimize the amount and type of heparin or heparan sulfate produced by the cell lines.

Genetic modifications suitable in mastocytoma cells, MST cells, and basophil neoplastic cell lines herein increase the amount of heparin and/or heparan sulfate, decrease the amount of contaminating substances, and alter the properties of the heparin and/or heparan sulfate produced by the cell lines. In some cases, genetically modified cell lines are deficient in one or more of chondroitin sulfate synthase 1 (CHSY1), chondroitin sulfate N-acetylgalactosaminyltransferase 2 (CSGALNACT2), chondroitin sulfate N-acetylgalactosaminyltransferase 1 (CSGALNACT1), heparan sulfate 2-O-sulfotransferase (Hs2st), and heparan sulfate C5-epimerase (GLCE). In some cases, the genetically modified cell line is deficient for a heparan sulfate catabolic enzyme. Non-limiting exemplary heparan sulfate catabolic enzyme comprise one or more of heparanase (HPSE), beta-glucoronidase (GUSB), sulfamidase (SGSH), heparan alpha-glucosaminide N-acetyltransferase (HGNAT), uronate-2-O-sulfatases (IDS and GDS), alpha-L-iduronidase (IDUA), and heparan sulfate 6-O-endosulfatase (SULF1-2). In addition, the genetically modified cell lines are contemplated to overexpresses one or more of heparanase, a protease, a heparan sulfate copolymerase, a sulfotransferase, an endosulfatase, and a proteoglycan protein core. Non-limiting exemplary proteases comprise one or more of matrix metalloproteases (MMP), MMP2, MMP3, MMP7, MMP9, MT1-MMP, MT3-MMP, ADAM17, ADAMTS1 and ADAMTS4, as well as trypsin, and chymotrypsin. Exemplary proteoglycan protein core include but are not limited to serglycin, a syndecan, a glypican, CD44 isoforms (CD44E), perlecan, collagen XVIII, and agrin, and recombinant versions thereof. In some cases, serglycin comprises a human serglycin, a pig serglycin, a mouse serglycin. In addition, it is contemplated that the number of GAG attachment sites in the serglycin is modified. Exemplary syndecans include but are not limited to a syndecan selected from the group consisting of syndecan-1, syndecan-2, syndecan-3, syndecan-4, and syndecan ectodomains. Non-limiting examples of glypicans include glypican 1, glypican 2, glypican 3, glypican 4, glypican 5, and glypican 6 and ectodomains.

Additional genetic modifications of mastocytoma cells, MST cells, and basophil neoplastic cell lines are provided in Table 1 and Table 2 below.

TABLE 1
Genetic Modifications for Cellular Production
of Heparin and Heparan Sulfate

| Gene KO | Gene KI | Enzymes |
|---|---|---|
| Chsy1 | | CS deficient |
| Chsy1 | | CS deficient |
| Hs2st | | HS 2-O-sulfation deficient |
| Chsy1 | | CS deficient |
| Glce | | HS epimerization deficient |
| Chsy1 | | CS deficient |
| Ndst1 | | NDST1-sulfation deficient |
| Chsy1 | | CS deficient |
| Ndst2 | | NDST2-sulfation deficient |
| Chsy1 | | CS deficient |
| Ndst1 | | NDST1-sulfation deficient |
| Ndst2 | | NDST2-sulfation deficient |
| Chsy1 | | CS deficient |
| Ndst3 | | NDST3-sulfation deficient |
| Chsy1 | | CS deficient |
| Ndst4 | | NDST4-sulfation deficient |
| Chsy1 | | CS deficient |
| Sulf1 | | Sulfatase 1 deficient |
| Chsy1 | | CS deficient |
| Sulf2 | | Sulfatase 2 deficient |
| Chsy1 | | CS deficient |
| Sulf1/2 | | Sulfatase 1 deficient |
| | | Sulfatase 2 deficient |
| Chsy1 | Ndst3 | CS deficient |
| Ndst1 | | NDST1-sulfation deficient |
| Ndst2 | | NDST2-sulfation deficient |
| | | NDST3-sulfation Added |
| Chsy1 | Ndst4 | CS deficient |
| Ndst1 | | NDST1-sulfation deficient |
| Ndst2 | | NDST2-sulfation deficient |
| | | NDST4-sulfation Added |
| Chsy1 | Hs6st1 | CS deficient |
| | | HS6ST1-sulfation Added |
| Chsy1 | Hs6st2 | CS deficient |
| | | HS6ST2-sulfation Added |
| Chsy1 | Hs6st3 | CS deficient |
| | | HS6ST3-sulfation Added |
| Chsy1 | Hs6st1 | CS deficient |
| | Hs6st2 | HS6ST1-sulfation Added |
| | | HS6ST2-sulfation Added |
| Chsy1 | Hs3st1 | CS deficient |
| | | HS3ST1-sulfation Added |
| Chsy1 | Hs3st2 | CS deficient |
| | | HS3ST-2sulfation Added |
| Chsy1 | Hs3st3a | CS deficient |
| | | HS3ST3a-sulfation Added |
| Chsy1 | Hs3st3b | CS deficient |
| | | HS3ST3b-sulfation Added |
| Chsy1 | Hs3st4 | CS deficient |
| | | HS3ST4-sulfation Added |
| Chsy1 | Hs3st5 | CS deficient |
| | | HS3ST5-sulfation Added |
| Chsy1 | Hs3st6 | CS deficient |
| | | HS 3ST6-sulfation Added |
| Chsy1 | Hs6st1 | CS deficient |
| | Hs6st2 | HS6ST1-sulfation Added |
| | Hs3st1 | HS6ST2-sulfation Added |
| | | HS3ST1-sulfation Added |
| Chsy1 | Hs6st1 | CS deficient |
| | Hs6st2 | HS6ST1-sulfation Added |
| | Hs3st2 | HS6ST2-sulfation Added |
| | | HS3ST2-sulfation Added |
| Chsy1 | Hs6st1 | CS deficient |
| | Hs6st2 | HS6ST1-sulfation Added |
| | Hs3st3a or 3b | HS6ST2-sulfation Added |
| | | HS3ST3-sulfation Added |
| Chsy1 | Hs6st1 | CS deficient |
| | Hs6st2 | HS6ST1-sulfation Added |
| | Hs3st4 | HS6ST2-sulfation Added |
| | | HS3ST4-sulfation Added |
| Chsy1 | Hs6st1 | CS deficient |
| | Hs6st2 | HS6ST1-sulfation Added |
| | Hs3st5 | HS6ST2-sulfation Added |
| | | HS3ST5-sulfation Added |
| Chsy1 | Hs6st1 | CS deficient |
| | Hs6st2 | HS6ST1-sulfation Added |
| | Hs3st6 | HS6ST2-sulfation Added |
| | | HS3ST6-sulfation Added |
| Chsy1 | Ndst3 | CS deficient |
| Ndst1 | Hs6st1 | NDST1-sulfation deficient |
| Ndst2 | | NDST2-sulfation deficient |
| | | NDST3-sulfation Added |
| | | HS6ST1-sulfation Added |
| Chsy1 | Ndst3 | CS deficient |
| Ndst1 | Hs6st2 | NDST1-sulfation deficient |
| Ndst2 | | NDST2-sulfation deficient |
| | | NDST3-sulfation Added |
| | | HS6ST2-sulfation Added |
| Chsy1 | Ndst3 | CS deficient |
| Ndst1 | Hs6st3 | NDST1-sulfation deficient |
| Ndst2 | | NDST2-sulfation deficient |
| | | NDST3-sulfation Added |
| | | HS6ST3-sulfation Added |
| Chsy1 | Ndst4 | CS deficient |
| Ndst1 | Hs6st1 | NDST1-sulfation deficient |
| Ndst2 | | NDST2-sulfation deficient |
| | | NDST4-sulfation Added |
| | | HS6ST1-sulfation Added |
| Chsy1 | Ndst4 | CS deficient |
| Ndst1 | Hs6st2 | NDST1-sulfation deficient |
| Ndst2 | | NDST2-sulfation deficient |
| | | NDST4-sulfation Added |
| | | HS6ST2-sulfation Added |
| Chsy1 | Ndst4 | CS deficient |
| Ndst1 | Hs6st3 | NDST1-sulfation deficient |
| Ndst2 | | NDST2-sulfation deficient |
| | | NDST4-sulfation Added |
| | | HS6ST3-sulfation Added |
| Chsy1 | Ndst3 | CS deficient |
| Ndst1 | Hs3st1 | NDST1-sulfation deficient |
| Ndst2 | | NDST2-sulfation deficient |
| | | NDST3-sulfation Added |
| | | HS3ST1-sulfation Added |
| Chsy1 | Ndst3 | CS deficient |
| Ndst1 | Hs3st2 | NDST1-sulfation deficient |
| Ndst2 | | NDST2-sulfation deficient |
| | | NDST3-sulfation Added |
| | | HS3ST2-sulfation Added |
| Chsy1 | Ndst3 | CS deficient |
| Ndst1 | 3a or 3b | NDST1-sulfation deficient |
| Ndst2 | | NDST2-sulfation deficient |
| | | NDST3-sulfation Added |
| | | HS3ST3-sulfation Added |
| Chsy1 | Ndst3 | CS deficient |
| Ndst1 | Hs3st4 | NDST1-sulfation deficient |
| Ndst2 | | NDST2-sulfation deficient |
| | | NDST3-sulfation Added |
| | | HS3ST4-sulfation Added |
| Chsy1 | Ndst3 | CS deficient |
| Ndst1 | Hs3st5 | NDST1-sulfation deficient |
| Ndst2 | | NDST2-sulfation deficient |
| | | NDST3-sulfation Added |
| | | HS3ST5-sulfation Added |
| Chsy1 | Ndst3 | CS deficient |
| Ndst1 | Hs3st6 | NDST1-sulfation deficient |
| Ndst2 | | NDST2-sulfation deficient |
| | | NDST3-sulfation Added |
| | | HS3ST6-sulfation Added |
| Chsy1 | Ndst4 | CS deficient |
| Ndst1 | Hs3st1 | NDST1-sulfation deficient |
| Ndst2 | | NDST2-sulfation deficient |
| | | NDST4-sulfation Added |
| | | HS3ST1-sulfation Added |
| Chsy1 | Ndst4 | CS deficient |
| Ndst1 | Hs3st2 | NDST1-sulfation deficient |
| Ndst2 | | NDST2-sulfation deficient |
| | | NDST4-sulfation Added |
| | | HS3ST2-sulfation Added |
| Chsy1 | Ndst4 | CS deficient |
| Ndst1 | Hs3st3a or 3b | NDST1-sulfation deficient |
| Ndst2 | | NDST2-sulfation deficient |
| | | NDST4-sulfation Added |
| | | HS3ST3-sulfation Added |
| Chsy1 | Ndst4 | CS deficient |
| Ndst1 | Hs3st4 | NDST1-sulfation deficient |
| Ndst2 | | NDST2-sulfation deficient |
| | | NDST4-sulfation Added |
| | | HS3ST4-sulfation Added |
| Chsy1 | Ndst4 | CS deficient |
| Ndst1 | Hs3st5 | NDST1-sulfation deficient |
| Ndst2 | | NDST2-sulfation deficient |
| | | NDST4-sulfation Added |
| | | HS3ST5-sulfation Added |
| Chsy1 | Ndst4 | CS deficient |
| Ndst1 | Hs3st6 | NDST1-sulfation deficient |
| Ndst2 | | NDST2-sulfation deficient |
| | | NDST4-sulfation Added |
| | | HS3ST6-sulfation Added |
| Chsy1 | Ndst3 | CS deficient |
| Ndst1 | Hs6st1/2 | NDST1-sulfation deficient |
| Ndst2 | Hs3st1 | NDST2-sulfation deficient |
| | | NDST3-sulfation Added |
| | | HS6ST1/2-sulfation Added |
| | | HS3ST1-sulfation Added |
| Chsy1 | Ndst3 | CS deficient |
| Ndst1 | Hs6st1/2 | NDST1-sulfation deficient |
| Ndst2 | Hs3st2 | NDST2-sulfation deficient |
| | | NDST3-sulfation Added |
| | | HS6ST1/2-sulfation Added |
| | | HS3ST2-sulfation Added |
| Chsy1 | Ndst3 | CS deficient |
| Ndst1 | Hs6st1/2 | NDST1-sulfation deficient |
| Ndst2 | Hs3st3a or 3b | NDST2-sulfation deficient |
| | | NDST3-sulfation Added |
| | | HS6ST1/2-sulfation Added |
| | | HS3ST3-sulfation Added |
| Chsy1 | Ndst3 | CS deficient |
| Ndst1 | Hs6st1/2 | NDST1-sulfation deficient |
| Ndst2 | Hs3stT4 | NDST2-sulfation deficient |
| | | NDST3-sulfation Added |
| | | HS6ST1/2-sulfation Added |
| | | HS3ST4-sulfation Added |
| Chsy1 | Ndst3 | CS deficient |
| Ndst1 | Hs6st1/2 | NDST1-sulfation deficient |
| Ndst2 | Hs3st5 | NDST2-sulfation deficient |
| | | NDST3-sulfation Added |
| | | HS6ST1/2-sulfation Added |
| | | HS3ST5-sulfation Added |
| Chsy1 | Ndst3 | CS deficient |
| Ndst1 | Hs6st1/2 | NDST1-sulfation deficient |
| Ndst2 | Hs3st6 | NDST2-sulfation deficient |
| | | NDST3-sulfation Added |
| | | HS6ST1/2-sulfation Added |
| | | HS3ST6-sulfation Added |
| Chsy1 | Ndst4 | CS deficient |
| Ndst1 | Hs6stT1/2 | NDST1-sulfation deficient |
| Ndst2 | Hs3st1 | NDST2-sulfation deficient |
| | | NDST4-sulfation Added |
| | | HS6ST1/2-sulfation Added |
| | | HS3ST1-sulfation Added |
| Chsy1 | Ndst3 | CS deficient |
| Ndst1 | Hs6st1/2 | NDST1-sulfation deficient |
| Ndst2 | Hs3st2 | NDST2-sulfation deficient |
| | | NDST4-sulfation Added |
| | | HS6ST1/2-sulfation Added |
| | | HS3ST2-sulfation Added |
| Chsy1 | Ndst3 | CS deficient |
| Ndst1 | Hs6st1/2 | NDST1-sulfation deficient |
| Ndst2 | Hs3st3a or 3b | NDST2-sulfation deficient |
| | | NDST4-sulfation Added |
| | | HS6ST1/2-sulfation Added |
| | | HS3ST3-sulfation Added |
| Chsy1 | Ndst3 | CS deficient |
| Ndst1 | Hs6st1/2 | NDST1-sulfation deficient |
| Ndst2 | HSs3st4 | NDST2-sulfation deficient |
| | | NDST4-sulfation Added |
| | | HS6ST1/2-sulfation Added |
| | | HS3ST4-sulfation Added |
| Chsy1 | Ndst3 | CS deficient |
| Ndst1 | Hs6st1/2 | NDST1-sulfation deficient |
| Ndst2 | Hs3st5 | NDST2-sulfation deficient |
| | | NDST4-sulfation Added |
| | | HS6ST1/2-sulfation Added |
| | | HS3ST5-sulfation Added |
| Chsy1 | Ndst3 | CS deficient |
| Ndst1 | Hs6st1/2 | NDST1-sulfation deficient |
| Ndst2 | Hs3st6 | NDST2-sulfation deficient |
| | | NDST4-sulfation Added |
| | | HS6ST1/2-sulfation Added |
| | | HS3ST6-sulfation Added |
| Chsy1 | Ndst1 | CS deficient |
| | | NDST1 over-sulfation |
| Chsy1 | Ndst2 | CS deficient |
| | | NDST2 over-sulfation |
| Chsy1 | Ndst1 & 2 | CS deficient |
| | | NDST1 & 2 over-sulfation |
| Chsy1 | Ndst1 | CS deficient |
| | Hs2st | NDST over-sulfation |
| | | HS2ST over-sulfation |
| Chsy1 | Ndst2 | CS deficient |
| | Hs2st | NDST2 over-sulfation |
| | | HS2ST over-sulfation |
| Chsy1 | Ndst1 & 2 | CS deficient |
| | Hs2st | NDST1 & 2 over-sulfation |
| | | HS2ST over-sulfation |
| Chsy1 | Ndst1 | CS deficient |
| | Glce | NDST1 over-sulfation |
| | | Epimerase over-epimerization |
| Chsy1 | Ndst2 | CS deficient |
| | Glce | NDST2 over-sulfation |
| | | Epimerase over-epimerization |
| Chsy1 | Ndst1 & 2 | CS deficient |
| | Glce | NDST1 & 2 over-sulfation |
| | | Epimerase over-epimerization |
| Chsy1 | Ndst1 | CS deficient |
| | Hs2st | NDST1 over-sulfation |
| | Glce | HS2ST over-sulfation |
| | | Epimerase over-epimerization |
| Chsy1 | Ndst2 | CS deficient |
| | Hs2st | NDST2 over-sulfation |
| | Glce | HS2ST over-sulfation |
| | | Epimerase over-epimerization |
| Chsy1 | Ndst1 & 2 | CS deficient |
| | Hs2st | NDST1 & 2 over-sulfation |
| | Glce | HS2ST over-sulfation |
| | | Epimerase over-epimerization |
| Chsy1 | Ndst1 | CS deficient |
| | Hs6st 1, 2 or 1 & 2 | NDST1 over-sulfation |
| | | HS6ST over-sulfation |
| Chsy1 | Ndst2 | CS deficient |
| | Hs6st 1, 2 or 1 & 2 | NDST2 over-sulfation |
| Chsy1 | Ndst1 & 2 | CS deficient |
| | Hs6st 1, 2 or 1 & 2 | NDST1 & 2 over-sulfation |
| Chsy1 | Ndst1 | CS deficient |
| | HS2st | NDST over-sulfation |
| | Hs6st 1, 2 or 1 & 2 | HS2ST over-sulfation |
| | | HS6ST over-sulfation |
| Chsy1 | Ndst2 | CS deficient |
| | Hs2st | NDST2 over-sulfation |
| | Hs6st 1, 2 or 1 & 2 | HS2ST over-sulfation |
| Chsy1 | Ndst1 & 2 | CS deficient |
| | Hs2st | NDST1 & 2 over-sulfation |
| | Hs6st 1, 2 or 1 & 2 | HS2ST over-sulfation |
| Chsy1 | Ndst1 | CS deficient |
| | Glce | NDST1 over-sulfation |
| | Hs6st 1, 2 or 1 & 2 | Epimerase over-epimerization |
| Chsy1 | Ndst2 | CS deficient |
| | Glce | NDST2 over-sulfation |
| | Hs6st 1, 2 or 1 & 2 | Epimerase over-epimerization |
| Chsy1 | Ndst1 & 2 | CS deficient |
| | Glce | NDST1 & 2 over-sulfation |
| | Hs6st 1, 2 or 1 & 2 | Epimerase over-epimerization |
| Chsy1 | Ndst1 | CS deficient |
| | Hs2st | NDST over-sulfation |
| | Glce | HS2ST over-sulfation |
| | Hs6st 1, 2 or 1 & 2 | Epimerase over-epimerization |
| Chsy1 | Ndst2 | CS deficient |
| | Hs2st | NDST2 over-sulfation |
| | Glce | HS2ST over-sulfation |
| | Hs6st 1, 2 or 1 & 2 | Epimerase over-epimerization |
| Chsy1 | Ndst1 & 2 | CS deficient |
| | Hs2st | NDST1 & 2 over-sulfation |
| | Glce | HS2ST over-sulfation |
| | Hs6st 1, 2 or 1 & 2 | Epimerase over-epimerization |
| | | HS6ST over-sulfation |
| Chsy1 | Ndst1 | CS deficient |
| | Hs3st | NDST1 over-sulfation |
| | | HS3ST over-sulfation |
| Chsy1 | Ndst2 | CS deficient |
| | Hs3st | NDST2 over-sulfation |
| | | HS3ST over-sulfation |
| Chsy1 | Ndst1 & 2 | CS deficient |
| | Hs3st | NDST1 & 2 over-sulfation |
| | | HS3ST over-sulfation |
| Chsy1 | Ndst1 | CS deficient |
| | Hs2st | NDST over-sulfation |
| | Hs3st | HS2ST over-sulfation |
| | | HS3ST over-sulfation |
| Chsy1 | Ndst2 | CS deficient |
| | Hs2st | NDST2 over-sulfation |
| | Hs3st | HS2ST over-sulfation |
| | | HS3ST over-sulfation |
| Chsy1 | Ndst1 & 2 | CS deficient |
| | Hs2st | NDST1 & 2 over-sulfation |
| | Hs3st | HS2ST over-sulfation |
| | | HS3ST over-sulfation |
| Chsy1 | Ndst1 | CS deficient |
| | Glce | NDST1 over-sulfation |
| | Hs3st | Epimerase over-epimerization |
| | | HS3ST over-sulfation |
| Chsy1 | Ndst2 | CS deficient |
| | Glce | NDST2 over-sulfation |
| | Hs3st | Epimerase over-epimerization |
| | | HS3ST over-sulfation |
| Chsy1 | Ndst1 & 2 | CS deficient |
| | Glce | NDST1 & 2 over-sulfation |
| | Hs3st | Epimerase over-epimerization |
| | | HS3ST over-sulfation |
| Chsy1 | Ndst1 | CS deficient |
| | Hs2st | NDST over-sulfation |
| | Glce | HS2ST over-sulfation |
| | Hs3st | Epimerase over-epimerization |
| | | HS3ST over-sulfation |
| Chsy1 | Ndst2 | CS deficient |
| | Hs2st | NDST2 over-sulfation |
| | Glce | HS2ST over-sulfation |
| | Hs3st | Epimerase over-epimerization |
| | | HS3ST over-sulfation |
| Chsy1 | Ndst1 & 2 | CS deficient |
| | Hs2st | NDST1 & 2 over-sulfation |
| | Glce | HS2ST over-sulfation |
| | Hs3st | Epimerase over-epimerization |
| | | HS3ST over-sulfation |
| Chsy1 | Ndst1 | CS deficient |
| | Hs6st 1, 2 or 1 & 2 | NDST1 over-sulfation |
| | Hs3st | HS6ST over-sulfation |
| | | HS3ST over-sulfation |
| Chsy1 | Ndst2 | CS deficient |
| | Hs6st 1, 2 or 1 & 2 | NDST2 over-sulfation |
| | Hs3st | HS3ST over-sulfation |
| | | HS3ST over-sulfation |
| Chsy1 | Ndst1 & 2 | CS deficient |
| | Hs6st 1, 2 or 1 & 2 | NDST1 & 2 over-sulfation |
| | Hs3st | HS3ST over-sulfation |
| Chsy1 | Ndst1 | CS deficient |
| | HS2st | NDST over-sulfation |
| | Hs6st 1, 2 or 1 & 2 | HS2ST over-sulfation |
| | Hs3st | HS6ST over-sulfation |
| | | HS3ST over-sulfation |
| Chsy1 | Ndst2 | CS deficient |
| | Hs2st | NDST2 over-sulfation |
| | Hs6st 1, 2 or 1 & 2 | HS2ST over-sulfation |
| | Hs3st | HS3ST over-sulfation |
| Chsy1 | Ndst1 & 2 | CS deficient |
| | Hs2st | NDST1 & 2 over-sulfation |
| | Hs6st 1, 2 or 1 & 2 | HS2ST over-sulfation |
| | Hs3st | HS3ST over-sulfation |
| Chsy1 | Ndst1 | CS deficient |
| | Glce | NDST1 over-sulfation |
| | Hs6st 1, 2 or 1 & 2 | Epimerase over-epimerization |
| | Hs3st | HS3ST over-sulfation |
| Chsy1 | Ndst2 | CS deficient |
| | Glce | NDST2 over-sulfation |
| | Hs6st 1, 2 or 1 & 2 | Epimerase over-epimerization |
| | Hs3st | HS3ST over-sulfation |
| Chsy1 | Ndst1 & 2 | CS deficient |
| | Glce | NDST1 & 2 over-sulfation |
| | Hs6st 1, 2 or 1 & 2 | Epimerase over-epimerization |
| | Hs3st | HS3ST over-sulfation |
| Chsy1 | Ndst1 | CS deficient |
| | Hs2st | NDST over-sulfation |
| | Glce | HS2ST over-sulfation |
| | Hs6st 1, 2 or 1 & 2 | Epimerase over-epimerization |
| | Hs3st | HS3ST over-sulfation |
| Chsy1 | Ndst2 | CS deficient |
| | Hs2st | NDST2 over-sulfation |
| | Glce | HS2ST over-sulfation |
| | Hs6st 1, 2 or 1 & 2 | Epimerase over-epimerization |
| | Hs3st | HS3ST over-sulfation |
| Chsy1 | Ndst1 & 2 | CS deficient |
| | Hs2st | NDST1 & 2 over-sulfation |
| | Glce | HS2ST over-sulfation |
| | Hs6st 1, 2 or 1 & 2 | Epimerase over-epimerization |
| | Hs3st | HS6ST over-sulfation |
| | | HS3ST over-sulfation |
| Chsy1 | Ndst1 | CS deficient |
| | Srgn | NDST1 over-sulfation |
| | | Increased expression of serglycin |
| Chsy1 | Ndst2 | CS deficient |
| | Srgn | NDST2 over-sulfation |
| | | Increased expression of serglycin |
| Chsy1 | Ndst1 & 2 | CS deficient |
| | Srgn | NDST1 & 2 over-sulfation |
| | | Increased expression of serglycin |
| Chsy1 | Ndst1 | CS deficient |
| | Hs2st | NDST over-sulfation |
| | Srgn | HS2ST over-sulfation |
| | | Increased expression of serglycin |
| Chsy1 | Ndst2 | CS deficient |
| | Hs2st | NDST2 over-sulfation |
| | Srgn | HS2ST over-sulfation |
| | | Increased expression of serglycin |
| Chsy1 | Ndst1 & 2 | CS deficient |
| | Hs2st | NDST1 & 2 over-sulfation |
| | Srgn | HS2ST over-sulfation |
| | | Increased expression of serglycin |
| Chsy1 | Ndst1 | CS deficient |
| | Glce | NDST1 over-sulfation |
| | Srgn | Epimerase over-epimerization |
| | | Increased expression of serglycin |
| Chsy1 | Ndst2 | CS deficient |
| | Glce | NDST2 over-sulfation |
| | Srgn | Epimerase over-epimerization |
| | | Increased expression of serglycin |
| Chsy1 | Ndst1 & 2 | CS deficient |
| | Glce | NDST1 & 2 over-sulfation |
| | Srgn | Epimerase over-epimerization |
| | | Increased expression of serglycin |
| Chsy1 | Ndst1 | CS deficient |
| | Hs2st | NDST over-sulfation |
| | Glce | HS2ST over-sulfation |
| | Srgn | Epimerase over-epimerization |
| | | Increased expression of serglycin |
| Chsy1 | Ndst2 | CS deficient |
| | Hs2st | NDST2 over-sulfation |
| | Glce | HS2ST over-sulfation |
| | Srgn | Epimerase over-epimerization |
| | | Increased expression of serglycin |
| Chsy1 | Ndst1 & 2 | CS deficient |
| | Hs2st | NDST1 & 2 over-sulfation |
| | Glce | HS2ST over-sulfation |
| | Srgn | Epimerase over-epimerization |
| | | Increased expression of serglycin |
| Chsy1 | Ndst1 | CS deficient |
| | Hs6st 1, 2 or 1 & 2 | NDST1 over-sulfation |
| | Srgn | HS6ST over-sulfation |
| | | Increased expression of serglycin |
| Chsy1 | Ndst2 | CS deficient |
| | Hs6st 1, 2 or 1 & 2 | NDST2 over-sulfation |
| | Srgn | HS6ST over-sulfation |
| | | Increased expression of serglycin |
| Chsy1 | Ndst1 & 2 | CS deficient |
| | Hs6st 1, 2 or 1 & 2 | NDST1 & 2 over-sulfation |
| | Srgn | HS6ST over-sulfation |
| | | Increased expression of serglycin |
| Chsy1 | Ndst1 | CS deficient |
| | HS2st | NDST over-sulfation |
| | Hs6st 1, 2 or 1 & 2 | HS2ST over-sulfation |
| | Srgn | HS6ST over-sulfation |
| | | Increased expression of serglycin |
| Chsy1 | Ndst2 | CS deficient |
| | Hs2st | NDST2 over-sulfation |
| | Hs6st 1, 2 or 1 & 2 | HS2ST over-sulfation |
| | Srgn | Increased expression of serglycin |
| Chsy1 | Ndst1 & 2 | CS deficient |
| | Hs2st | NDST1 & 2 over-sulfation |
| | Hs6st 1, 2 or 1 & 2 | HS2ST over-sulfation |
| | Srgn | Increased expression of serglycin |
| Chsy1 | Ndst1 | CS deficient |
| | Glce | NDST1 over-sulfation |
| | Hs6st 1, 2 or 1 & 2 | Epimerase over-epimerization |
| | Srgn | HS6ST over-sulfation |
| | | Increased expression of serglycin |
| Chsy1 | Ndst2 | CS deficient |
| | Glce | NDST2 over-sulfation |
| | Hs6st 1, 2 or 1 & 2 | Epimerase over-epimerization |
| | Srgn | HS6ST over-sulfation |
| | | Increased expression of serglycin |
| Chsy1 | Ndst1 & 2 | CS deficient |
| | Glce | NDST1 & 2 over-sulfation |
| | Hs6st 1, 2 or 1 & 2 | Epimerase over-epimerization |
| | Srgn | HS6ST over-sulfation |
| | | Increased expression of serglycin |
| Chsy1 | Ndst1 | CS deficient |
| | Hs2st | NDST over-sulfation |
| | Glce | HS2ST over-sulfation |
| | Hs6st 1, 2 or 1 & 2 | Epimerase over-epimerization |
| | Srgn | HS6ST over-sulfation |
| | | Increased expression of serglycin |
| Chsy1 | Ndst2 | CS deficient |
| | Hs2st | NDST2 over-sulfation |
| | Glce | HS2ST over-sulfation |
| | Hs6st 1, 2 or 1 & 2 | Epimerase over-epimerization |
| | Srgn | HS6ST over-sulfation |
| | | Increased expression of serglycin |
| Chsy1 | Ndst1 & 2 | CS deficient |
| | Hs2st | NDST1 & 2 over-sulfation |
| | Glce | HS2ST over-sulfation |
| | Hs6st 1, 2 or 1 & 2 | Epimerase over-epimerization |
| | Srgn | HS6ST over-sulfation |
| | | Increased expression of serglycin |
| Chsy1 | Ndst1 | CS deficient |
| | Hs3st | NDST1 over-sulfation |
| | Srgn | HS3ST over-sulfation |
| | | Increased expression of serglycin |
| Chsy1 | Ndst2 | CS deficient |
| | Hs3st | NDST2 over-sulfation |
| | Srgn | HS3ST over-sulfation |
| | | Increased expression of serglycin |
| Chsy1 | Ndst1 & 2 | CS deficient |
| | Hs3st | NDST1 & 2 over-sulfation |
| | Srgn | HS3ST over-sulfation |
| | | Increased expression of serglycin |
| Chsy1 | Ndst1 | CS deficient |
| | Hs2st | NDST over-sulfation |
| | Hs3st | HS2ST over-sulfation |
| | Srgn | HS3ST over-sulfation |
| | | Increased expression of serglycin |
| Chsy1 | Ndst2 | CS deficient |
| | Hs2st | NDST2 over-sulfation |
| | Hs3st | HS2ST over-sulfation |
| | Srgn | HS3ST over-sulfation |
| | | Increased expression of serglycin |
| Chsy1 | Ndst1 & 2 | CS deficient |
| | Hs2st | NDST1 & 2 over-sulfation |
| | Hs3st | HS2ST over-sulfation |
| | Srgn | HS3ST over-sulfation |
| | | Increased expression of serglycin |
| Chsy1 | Ndst1 | CS deficient |
| | Glce | NDST1 over-sulfation |
| | Hs3st | Epimerase over-epimerization |
| | Srgn | HS3ST over-sulfation |
| | | Increased expression of serglycin |
| Chsy1 | Ndst2 | CS deficient |
| | Glce | NDST2 over-sulfation |
| | Hs3st | Epimerase over-epimerization |
| | Srgn | HS3ST over-sulfation |
| | | Increased expression of serglycin |
| Chsy1 | Ndst1 & 2 | CS deficient |
| | Glce | NDST1 & 2 over-sulfation |
| | Hs3st | Epimerase over-epimerization |
| | Srgn | HS3ST over-sulfation |
| | | Increased expression of serglycin |
| Chsy1 | Ndst1 | CS deficient |
| | Hs2st | NDST over-sulfation |
| | Glce | HS2ST over-sulfation |
| | Hs3st | Epimerase over-epimerization |
| | Srgn | HS3ST over-sulfation |
| | | Increased expression of serglycin |
| Chsy1 | Ndst2 | CS deficient |
| | Hs2st | NDST2 over-sulfation |
| | Glce | HS2ST over-sulfation |
| | Hs3st | Epimerase over-epimerization |
| | Srgn | HS3ST over-sulfation |
| | | Increased expression of serglycin |
| Chsy1 | Ndst1 & 2 | CS deficient |
| | Hs2st | NDST1 & 2 over-sulfation |
| | Glce | HS2ST over-sulfation |
| | Hs3st | Epimerase over-epimerization |
| | Srgn | HS3ST over-sulfation |
| | | Increased expression of serglycin |
| Chsy1 | Ndst1 | CS deficient |
| | Hs6st 1, 2 or 1 & 2 | NDST1 over-sulfation |
| | Hs3st | HS6ST over-sulfation |
| | Srgn | HS3ST over-sulfation |
| | | Increased expression of serglycin |
| Chsy1 | Ndst2 | CS deficient |
| | Hs6st 1, 2 or 1 & 2 | NDST2 over-sulfation |
| | Hs3st | HS3ST over-sulfation |
| | Srgn | HS3ST over-sulfation |
| | | Increased expression of serglycin |
| Chsy1 | Ndst1 & 2 | CS deficient |
| | Hs6st 1, 2 or 1 & 2 | NDST1 & 2 over-sulfation |
| | Hs3st | HS6ST over-sulfation |
| | Srgn | HS3ST over-sulfation |
| | | Increased expression of serglycin |
| Chsy1 | Ndst1 | CS deficient |
| | HS2st | NDST over-sulfation |
| | Hs6st 1, 2 or 1 & 2 | HS2ST over-sulfation |
| | Hs3st | HS6ST over-sulfation |
| | Srgn | HS3ST over-sulfation |
| | | Increased expression of serglycin |
| Chsy1 | Ndst2 | CS deficient |
| | Hs2st | NDST2 over-sulfation |
| | Hs6st 1, 2 or 1 & 2 | HS6ST over-sulfation |
| | Hs3st | HS2ST over-sulfation |
| | Srgn | HS3ST over-sulfation |
| | | Increased expression of serglycin |
| Chsy1 | Ndst1 & 2 | CS deficient |
| | Hs2st | NDST1 & 2 over-sulfation |
| | Hs6st 1, 2 or 1 & 2 | HS2ST over-sulfation |
| | Hs3st | HS6ST over-expression |
| | Srgn | HS3ST over-sulfation |
| | | Increased expression of serglycin |
| Chsy1 | Ndst1 | CS deficient |
| | Glce | NDST1 over-sulfation |
| | Hs6st 1, 2 or 1 & 2 | Epimerase over-epimerization |
| | Hs3st | HS6ST over-expression |
| | Srgn | HS3ST over-sulfation |
| | | Increased expression of serglycin |
| Chsy1 | Ndst2 | CS deficient |
| | Glce | NDST2 over-sulfation |
| | Hs6st 1, 2 or 1 & 2 | Epimerase over-epimerization |
| | Hs3st | HS6ST over-expression |
| | Srgn | HS3ST over-sulfation |
| | | Increased expression of serglycin |
| Chsy1 | Ndst1 & 2 | CS deficient |
| | Glce | NDST1 & 2 over-sulfation |
| | Hs6st 1, 2 or 1 & 2 | Epimerase over-epimerization |
| | Hs3st | HS6ST oversulfation |
| | Srgn | HS3ST over-sulfation |
| | | Increased expression of serglycin |
| Chsy1 | Ndst1 | CS deficient |
| | Hs2st | NDST over-sulfation |
| | Glce | HS2ST over-sulfation |
| | Hs6st 1, 2 or 1 & 2 | Epimerase over-epimerization |
| | Hs3st | HS6ST over-sulfation |
| | Srgn | HS3ST over-sulfation |
| | | Increased expression of serglycin |
| Chsy1 | Ndst2 | CS deficient |
| | Hs2st | NDST2 over-sulfation |
| | Glce | HS2ST over-sulfation |
| | Hs6st 1, 2 or 1 & 2 | Epimerase over-epimerization |
| | Hs3st | HS6ST over-expression |
| | Srgn | HS3ST over-sulfation |
| | | Increased expression of serglycin |
| Chsy1 | Ndst1 & 2 | CS deficient |
| | Hs2st | NDST1 & 2 over-sulfation |
| | Glce | HS2ST over-sulfation |
| | Hs6st 1, 2 or 1 & 2 | Epimerase over-epimerization |
| | Hs3st | HS6ST over-sulfation |
| | Srgn | HS3ST over-sulfation |
| | | Increased expression of serglycin |
| Chsy1 | Ndst1 | CS deficient |
| | Srgn | NDST1 over-sulfation |
| | EXT2, 1, L3 | Increased expression of serglycin |
| | | HS over-polymerization |
| Chsy1 | Ndst2 | CS deficient |
| | Srgn | NDST2 over-sulfation |
| | EXT2, 1, L3 | Increased expression of serglycin |
| | | HS over-polymerization |
| Chsy1 | Ndst1 & 2 | CS deficient |
| | Srgn | NDST1 & 2 over-sulfation |
| | EXT2, 1, L3 | Increased expression of serglycin |
| | | HS over-polymerization |
| Chsy1 | Ndst1 | CS deficient |
| | Hs2st | NDST over-sulfation |
| | Srgn | HS2ST over-sulfation |
| | EXT2, 1, L3 | Increased expression of serglycin |
| | | HS over-polymerization |
| Chsy1 | Ndst2 | CS deficient |
| | Hs2st | NDST2 over-sulfation |
| | Srgn | HS2ST over-sulfation |
| | EXT2, 1, L3 | Increased expression of serglycin |
| | | HS over-polymerization |
| Chsy1 | Ndst1 & 2 | CS deficient |
| | Hs2st | NDST1 & 2 over-sulfation |
| | Srgn | HS2ST over-sulfation |
| | EXT2, 1, L3 | Increased expression of serglycin |
| | | HS over-polymerization |
| Chsy1 | Ndst1 | CS deficient |
| | Glce | NDST1 over-sulfation |
| | Srgn | Epimerase over-epimerization |
| | EXT2, 1, L3 | Increased expression of serglycin |
| | | HS over-polymerization |
| Chsy1 | Ndst2 | CS deficient |
| | Glce | NDST2 over-sulfation |
| | Srgn | Epimerase over-epimerization |
| | EXT2, 1, L3 | Increased expression of serglycin |
| | | HS over-polymerization |
| Chsy1 | Ndst1 & 2 | CS deficient |
| | Glce | NDST1 & 2 over-sulfation |
| | Srgn | Epimerase over-epimerization |
| | EXT2, 1, L3 | Increased expression of serglycin |
| | | HS over-polymerization |
| Chsy1 | Ndst1 | CS deficient |
| | Hs2st | NDST over-sulfation |
| | Glce | HS2ST over-sulfation |
| | Srgn | Epimerase over-epimerization |
| | EXT2, 1, L3 | Increased expression of serglycin |
| | | HS over-polymerization |
| Chsy1 | Ndst2 | CS deficient |
| | Hs2st | NDST2 over-sulfation |
| | Glce | HS2ST over-sulfation |
| | Srgn | Epimerase over-epimerization |
| | EXT2, 1, L3 | Increased expression of serglycin |
| | | HS over-polymerization |
| Chsy1 | Ndst1 & 2 | CS deficient |
| | Hs2st | NDST1 & 2 over-sulfation |
| | Glce | HS2ST over-sulfation |
| | Srgn | Epimerase over-epimerization |
| | EXT2, 1, L3 | Increased expression of serglycin |
| | | HS over-polymerization |
| Chsy1 | Ndst1 | CS deficient |
| | Hs6st 1, 2 or 1 & 2 | NDST1 over-sulfation |
| | Srgn | HS6ST over-sulfation |
| | EXT2, 1, L3 | Increased expression of serglycin |
| | | HS over-polymerization |
| Chsy1 | Ndst2 | CS deficient |
| | Hs6st 1, 2 or 1 & 2 | NDST2 over-sulfation |
| | Srgn | HS6ST over-sulfation |
| | EXT2, 1, L3 | Increased expression of serglycin |
| | | HS over-polymerization |
| Chsy1 | Ndst1 & 2 | CS deficient |
| | Hs6st 1, 2 or 1 & 2 | NDST1 & 2 over-sulfation |
| | Srgn | HS6ST over-sulfation |
| | EXT2, 1, L3 | Increased expression of serglycin |
| | | HS over-polymerization |
| Chsy1 | Ndst1 | CS deficient |
| | Hs2st | NDST1 over-sulfation |
| | Hs6st 1, 2 or 1 & 2 | HS2ST over-sulfation |
| | Srgn | HS6ST over-sulfation |
| | EXT2, 1, L3 | Increased expression of serglycin |
| | | HS over-polymerization |
| Chsy1 | Ndst2 | CS deficient |
| | Hs2st | NDST2 over-sulfation |
| | Hs6st 1, 2 or 1 & 2 | HS2ST over-sulfation |
| | Srgn | HS6ST over-sulfation |
| | EXT2, 1, L3 | Increased expression of serglycin |
| | | HS over-polymerization |
| Chsy1 | Ndst1 & 2 | CS deficient |
| | Hs2st | NDST1 & 2 over-sulfation |
| | Hs6st 1, 2 or 1 & 2 | HS2ST over-sulfation |
| | Srgn | HS6ST over-sulfation |
| | EXT2, 1, L3 | Increased expression of serglycin |
| | | HS over-polymerization |
| Chsy1 | Ndst1 | CS deficient |
| | Glce | NDST1 over-sulfation |
| | Hs6st 1, 2 or 1 & 2 | Epimerase over-epimerization |
| | Srgn | HS6ST over-sulfation |
| | EXT2, 1, L3 | Increased expression of serglycin |
| | | HS over-polymerization |
| Chsy1 | Ndst2 | CS deficient |
| | Glce | NDST2 over-sulfation |
| | Hs6st 1, 2 or 1 & 2 | Epimerase over-epimerization |
| | Srgn | HS6ST over-sulfation |
| | EXT2, 1, L3 | Increased expression of serglycin |
| | | HS over-polymerization |
| Chsy1 | Ndst1 & 2 | CS deficient |
| | Glce | NDST1 & 2 over-sulfation |
| | Hs6st 1, 2 or 1 & 2 | Epimerase over-epimerization |
| | Srgn | HS6ST over-sulfation |
| | EXT2, 1, L3 | Increased expression of serglycin |
| | | HS over-polymerization |
| Chsy1 | Ndst1 | CS deficient |
| | Hs2st | NDST over-sulfation |
| | Glce | HS2ST over-sulfation |
| | Hs6st 1, 2 or 1 & 2 | Epimerase over-epimerization |
| | Srgn | HS6ST over-sulfation |
| | EXT2, 1, L3 | Increased expression of serglycin |
| | | HS over-polymerization |
| Chsy1 | Ndst2 | CS deficient |
| | Hs2st | NDST2 over-sulfation |
| | Glce | HS2ST over-sulfation |
| | Hs6st 1, 2 or 1 & 2 | Epimerase over-epimerization |
| | Srgn | HS6ST over-sulfation |
| | EXT2, 1, L3 | Increased expression of serglycin |
| | | HS over-polymerization |
| Chsy1 | Ndst1 & 2 | CS deficient |
| | Hs2st | NDST1 & 2 over-sulfation |
| | Glce | HS2ST over-sulfation |
| | Hs6st 1, 2 or 1 & 2 | Epimerase over-epimerization |
| | Srgn | HS6ST over-sulfation |
| | EXT2, 1, L3 | Increased expression of serglycin |
| | | HS over-polymerization |
| Chsy1 | Ndst1 | CS deficient |
| | Hs3st | NDST1 over-sulfation |
| | Srgn | HS3ST over-sulfation |
| | EXT2, 1, L3 | Increased expression of serglycin |
| | | HS over-polymerization |
| Chsy1 | Ndst2 | CS deficient |
| | Hs3st | NDST2 over-sulfation |
| | Srgn | HS3ST over-sulfation |
| | EXT2, 1, L3 | Increased expression of serglycin |
| | | HS over-polymerization |
| Chsy1 | Ndst1 & 2 | CS deficient |
| | Hs3st | NDST1 & 2 over-sulfation |
| | Srgn | HS3ST over-sulfation |
| | EXT2, 1, L3 | Increased expression of serglycin |
| | | HS over-polymerization |
| Chsy1 | Ndst1 | CS deficient |
| | Hs2st | NDST over-sulfation |
| | Hs3st | HS2ST over-sulfation |
| | Srgn | HS3ST over-sulfation |
| | EXT2, 1, L3 | Increased expression of serglycin |
| | | HS over-polymerization |
| Chsy1 | Ndst2 | CS deficient |
| | Hs2st | NDST2 over-sulfation |
| | Hs3st | HS2ST over-sulfation |
| | Srgn | HS3ST over-sulfation |
| | EXT2, 1, L3 | Increased expression of serglycin |
| | | HS over-polymerization |
| Chsy1 | Ndst1 & 2 | CS deficient |
| | Hs2st | NDST1 & 2 over-sulfation |
| | Hs3st | HS2ST over-sulfation |
| | Srgn | HS3ST over-sulfation |
| | EXT2, 1, L3 | Increased expression of serglycin |
| | | HS over-polymerization |
| Chsy1 | Ndst1 | CS deficient |
| | Glce | NDST1 over-sulfation |
| | Hs3st | Epimerase over-epimerization |
| | Srgn | HS3ST over-sulfation |
| | EXT2, 1, L3 | Increased expression of serglycin |
| | | HS over-polymerization |
| Chsy1 | Ndst2 | CS deficient |
| | Glce | NDST2 over-sulfation |
| | Hs3st | Epimerase over-epimerization |
| | Srgn | HS3ST over-sulfation |
| | EXT2, 1, L3 | Increased expression of serglycin |
| | | HS over-polymerization |
| Chsy1 | Ndst1 & 2 | CS deficient |
| | Glce | NDST1 & 2 over-sulfation |
| | Hs3st | Epimerase over-epimerization |
| | Srgn | HS3ST over-sulfation |
| | EXT2, 1, L3 | Increased expression of serglycin |
| | | HS over-polymerization |
| Chsy1 | Ndst1 | CS deficient |
| | Hs2st | NDST over-sulfation |
| | Glce | HS2ST over-sulfation |
| | Hs3st | Epimerase over-epimerization |
| | Srgn | HS3ST over-sulfation |
| | EXT2, 1, L3 | Increased expression of serglycin |
| | | HS over-polymerization |
| Chsy1 | Ndst2 | CS deficient |
| | Hs2st | NDST2 over-sulfation |
| | Glce | HS2ST over-sulfation |
| | Hs3st | Epimerase over-epimerization |
| | Srgn | HS3ST over-sulfation |
| | EXT2, 1, L3 | Increased expression of serglycin |
| | | HS over-polymerization |
| Chsy1 | Ndst1 & 2 | CS deficient |
| | Hs2st | NDST1 & 2 over-sulfation |
| | Glce | HS2ST over-sulfation |
| | Hs3st | Epimerase over-epimerization |
| | Srgn | HS3ST over-sulfation |
| | EXT2, 1, L3 | Increased expression of serglycin |
| | | HS over-polymerization |
| Chsy1 | Ndst1 | CS deficient |
| | Hs6st 1, 2 or 1 & 2 | NDST1 over-sulfation |
| | Hs3st | HS6ST over-sulfation |
| | Srgn | HS3ST over-sulfation |
| | EXT2, 1, L3 | Increased expression of serglycin |
| | | HS over-polymerization |
| Chsy1 | Ndst2 | CS deficient |
| | Hs6st 1, 2 or 1 & 2 | NDST2 over-sulfation |
| | Hs3st | HS3ST over-sulfation |
| | Srgn | HS3ST over-sulfation |
| | EXT2, 1, L3 | Increased expression of serglycin |
| | | HS over-polymerization |
| Chsy1 | Ndst1 & 2 | CS deficient |
| | Hs6st 1, 2 or 1 & 2 | NDST1 & 2 over-sulfation |
| | Hs3st | HS3ST over-sulfation |
| | Srgn | Increased expression of serglycin |
| | EXT2, 1, L3 | HS over-polymerization |
| Chsy1 | Ndst1 | CS deficient |
| | HS2st | NDST over-sulfation |
| | Hs6st 1, 2 or 1 & 2 | HS2ST over-sulfation |
| | Hs3st | HS6ST over-sulfation |
| | Srgn | HS3ST over-sulfation |
| | EXT2, 1, L3 | Increased expression of serglycin |
| | | HS over-polymerization |
| Chsy1 | Ndst2 | CS deficient |
| | Hs2st | NDST2 over-sulfation |
| | Hs6st 1, 2 or 1 & 2 | HS2ST over-sulfation |
| | Hs3st | HS6ST over-sulfation |
| | Srgn | HS3ST over-sulfation |
| | EXT2, 1, L3 | Increased expression of serglycin |
| | | HS over-polymerization |
| Chsy1 | Ndst1 & 2 | CS deficient |
| | Hs2st | NDST1 & 2 over-sulfation |
| | Hs6st 1, 2 or 1 & 2 | HS2ST over-sulfation |
| | Hs3st | HS6ST over-sulfation |
| | Srgn | HS3ST over-sulfation |
| | EXT2, 1, L3 | Increased expression of serglycin |
| | | HS over-polymerization |
| Chsy1 | Ndst1 | CS deficient |
| | Glce | NDST1 over-sulfation |
| | Hs6st 1, 2 or 1 & 2 | Epimerase over-epimerization |
| | Hs3st | HS6ST over-sulfation |
| | Srgn | HS3ST over-sulfation |
| | EXT2, 1, L3 | Increased expression of serglycin |
| | | HS over-polymerization |
| Chsy1 | Ndst2 | CS deficient |
| | Glce | NDST2 over-sulfation |
| | Hs6st 1, 2 or 1 & 2 | Epimerase over-epimerization |
| | Hs3st | HS6ST over-sulfation |
| | Srgn | HS3ST over-sulfation |
| | EXT2, 1, L3 | Increased expression of serglycin |
| | | HS over-polymerization |
| Chsy1 | Ndst1 & 2 | CS deficient |
| | Glce | NDST1 & 2 over-sulfation |
| | Hs6st 1, 2 or 1 & 2 | Epimerase over-epimerization |
| | Hs3st | HS6ST over-sulfation |
| | Srgn | HS3ST over-sulfation |
| | EXT2, 1, L3 | Increased expression of serglycin |
| | | HS over-polymerization |
| Chsy1 | Ndst1 | CS deficient |
| | Hs2st | NDST over-sulfation |
| | Glce | HS2ST over-sulfation |
| | Hs6st 1, 2 or 1 & 2 | Epimerase over-epimerization |
| | Hs3st | HS6ST over-sulfation |
| | Srgn | HS3ST over-sulfation |
| | EXT2, 1, L3 | Increased expression of serglycin |
| | | HS over-polymerization |
| Chsy1 | Ndst2 | CS deficient |
| | Hs2st | NDST2 over-sulfation |
| | Glce | HS2ST over-sulfation |
| | Hs6st 1, 2 or 1 & 2 | Epimerase over-epimerization |
| | Hs3st | HS6ST over-sulfation |
| | Srgn | HS3ST over-sulfation |
| | EXT2, 1, L3 | Increased expression of serglycin |
| | | HS over-polymerization |
| Chsy1 | Ndst1 & 2 | CS deficient |
| | Hs2st | NDST1 & 2 over-sulfation |
| | Glce | HS2ST over-sulfation |
| | Hs6st 1, 2 or 1 & 2 | Epimerase over-epimerization |
| | Hs3st | HS6ST over-sulfation |
| | Srgn | HS3ST over-sulfation |
| | EXT2, 1, L3 | Increased expression of serglycin |
| | | HS over-polymerization |

TABLE 2
Genes for Genetically Modified Cells Lines Producing Heparin and Heparan Sulfate

| Gene (Accession | | |
|---|---|---|
| Number) | Protein Name | Enzymatic Activity |
| SLC26A2 | DTD ST sulfate anion | Transport of sulfate across plasma |
| (NP_000103) | transporter 1 | membrane |
| PAPSS1 (Y10387) | PAPS synthetase-1 (ATP | PAPS formation |
| | sulfurylase/APS kinase 1) | KI: increase sulfation |
| | | KO: decrease sulfation |
| PAPSS2 | PAPS synthetase-2 (ATP | PAPS formation |
| (AF074331) | sulfurylase/APS kinase 2) | KI: increase sulfation |
| | | KO: decrease sulfation |
| AB107958 | PAPS transporter | Import of PAPS into Golgi |
| | | KI: increase sulfation |
| | | KO: decrease sulfation |
| SLC35B2 | PAPS transporter 1 | Import of PAPS into Golgi |
| (Q8TB61.1) | | KI: increase sulfation |
| | | KO: decrease sulfation |
| SLC35B3 | PAPS transporter 2 | Import of PAPS into Golgi |
| (Q9H1N7.1) | | KI: increase sulfation |
| | | KO: decrease sulfation |
| SLC35D1 | UDP-GlcA transporter | Import of UDP-GlcA/UDP-GalNAc |
| (NP_055954) | | KI: increase GAG production |
| | | KO: decrease GAG production |
| SLC35A3 | UDP-GlcNAc transporter | Import of UDP-GlcNAc |
| (NP_036375) | | KI: increase GAG production |
| | | KO: decrease GAG production |
| SLC35A2 | UDP-Gal transporter | Import of UDP-Gal |
| (P78381) | | KI: increase GAG production |
| | | KO: decrease GAG production |
| UGDH | UDP glucose dehydrogenase | Conversion of UDP-Glc to UDP-GlcA |
| (AAC36095) | | KI: increase GAG production |
| | | KO: decrease GAG production |
| UXS | UDP-glucose decarboxylase | Conversion of UDP-GlcA to UDP-Xyl |
| (AAK85410) | | KI: increase GAG production |
| | | KO: decrease GAG production |
| XTI | Xylosyltransferase I | Attachment of Xylose to Ser of core protein |
| (AJ277441) | | KI: increase GAG production |
| | | KO: decrease GAG production |
| XTII | Xylosyltransferase II | Attachment of Xylose to Ser of core protein |
| (AJ277442) | | KI: increase GAG production |
| | | KO: decrease GAG production |
| XGALT-1 | Galactosyltransferase I | Addition of Galβ4 to Xylβ4Ser |
| (AB028600) | (β4GalT7) | KI: increase GAG production |
| | | KO: decrease GAG production |
| GALT-2 | Galactosyltransferase II | Addition of Galβ3 to Galβ4Xyl |
| (AY050570) | (β3GalT6) | KI: increase GAG production |
| | | KO: decrease GAG production |
| GLCAT-1 | Glucuronosyltransferase I | Addition of GlcAβ3 to Galβ3Galβ4Xyl |
| (AB009598) | | KI: increase GAG production |
| | | KO: decrease GAG production |
| EXTL1 | α-N- | Addition of GlcNAcα4 to Galβ3Galβ4Xyl |
| (AH007206) | acetylglucosaminyltransferase I | KI: increase GAG production |
| | | KO: decrease GAG production |
| EXTL2 | α-N- | Addition of GlcNAcα4 to Galβ3Galβ4Xyl |
| (AF000416) | acetylglucosaminyltransferase I | KI: increase GAG production |
| | | KO: decrease GAG production |
| EXTL3 | α-N- | Addition of GlcNAcα4 to Galβ3Galβ4Xyl |
| (AF001690) | acetylglucosaminyltransferase I | KI: increase GAG production |
| | | KO: decrease GAG production |
| EXT1 | Exostosin glycosyltransferase 1 | GlcNAcα4 & GlcAβ4 |
| (S79639) | | transferases: HS copolymerase |
| | | KI: increase GAG production |
| | | KO: decrease GAG production |
| EXT2 | Exostosin glycosyltransferase 2 | GlcNAcα4 & GlcAβ4 |
| (NM_000401) | | transferases: HS copolymerase |
| | | KI: increase GAG production |
| | | KO: decrease GAG production |
| NDST1 | N-deacetylase N-sulfotransferase | N-deacetylation and N-sulfation of GlcNAc |
| (U18918) | 1 | KI: increase N-sulfation |
| | | KO: decrease N-sulfation |
| NDST2 | N-deacetylase N-sulfotransferase | N-deacetylation and N-sulfation of GlcNAc |
| (NM_003635) | 2 | KI: increase N-sulfation |
| | | KO: decrease N-sulfation |
| NDST3 | N-deacetylase N-sulfotransferase | N-deacetylation and N-sulfation of GlcNAc |
| (AF074924) | 3 | KI: increase N-sulfation |
| | | KO: decrease N-sulfation |
| NDST4 | N-deacetylase N-sulfotransferase | N-deacetylation and N-sulfation of GlcNAc |
| (AB036429) | 4 | KI: increase N-sulfation |
| | | KO: decrease N-sulfation |
| C5EPI | C5 glucuronyl epimerase | Conversion of D-GlcA to L-IdoA |
| (XM_035390) | | KI: increase epimerization |
| | | KO: decrease epimerization |
| HS2ST | Heparan sulfate 2-O- | 2-O-sulfation of IdoA |
| (AB024568) | sulfotransferase | KI: increase 2-O-sulfation |
| | | KO: decrease 2-O-sulfation |
| HS6ST1 | Heparan sulfate glucosamine | 6-O-sulfation of glucosamine |
| (AB006179) | 6-O-sulfotransferase 1 | KI: increase 6-O-sulfation |
| | | KO: decrease 6-O-sulfation |
| HS6ST2 | Heparan sulfate glucosamine | 6-O-sulfation of glucosamine |
| (NM_147174) | 6-O-sulfotransferase 2 | KI: increase 6-O-sulfation |
| | | KO: decrease 6-O-sulfation |
| HS6ST2 | Heparan sulfate glucosamine | 6-O-sulfation of glucosamine |
| (NM_147175) | 6-O-sulfotransferase 2 short | KI: increase 6-O-sulfation |
| | | KO: decrease 6-O-sulfation |
| HS6ST3 | Heparan sulfate glucosamine | 6-O-sulfation of glucosamine |
| (AF539426) | 6-O-sulfotransferase 3 | KI: increase 6-O-sulfation |
| | | KO: decrease 6-O-sulfation |
| HS3ST1 | Heparan sulfate glucosamine | 3-O-sulfation of glucosamine |
| (AF019386) | 3-O-sulfotransferase 1 | (GlcAGlcNS(±6S) |
| | | KI: increase 3-O-sulfation |
| | | KO: decrease 3-O-sulfation |
| HS3ST2 | Heparan sulfate glucosamine | 3-O-sulfation of glucosamine in restricted |
| (AF105375) | 3-O-sulfotransferase 2 | domains |
| | | KI: increase 3-O-sulfation |
| | | KO: decrease 3-O-sulfation |
| HS3ST3A1 | Heparan sulfate glucosamine | 3-O-sulfation of glucosamine in restricted |
| (AF105376) | 3-O-sulfotransferase 3A | domains |
| | | KI: increase 3-O-sulfation |
| | | KO: decrease 3-O-sulfation |
| HS3ST3B1 | Heparan sulfate glucosamine | 3-O-sulfation of glucosamine in restricted |
| (AF105377) | 3-O-sulfotransferase 3B | domains |
| | | KI: increase 3-O-sulfation |
| | | KO: decrease 3-O-sulfation |
| HS3ST4 | Heparan sulfate glucosamine | 3-O-sulfation of glucosamine in restricted |
| (AF105378) | 3-O-sulfotransferase 4 | domains |
| HS3ST5 | Heparan sulfate glucosamine | 3-O-sulfation of glucosamine in restricted |
| (AF503292) | 3-O-sulfotransferase 5 | domains |
| | | KI: increase 3-O-sulfation |
| | | KO: decrease 3-O-sulfation |
| HS3ST6 | Heparan sulfate glucosamine | 3-O-sulfation of glucosamine in restricted |
| (AE00640) | 3-O-sulfotransferase 6 | domains |
| | | KI: increase 3-O-sulfation |
| | | KO: decrease 3-O-sulfation |
| SDC-1 | Syndecan-1 | Membrane spanning proteoglycan |
| (HS.82109) | | KI: increase GAG production |
| | | KO: decrease GAG production |
| SDC-2 | Syndecan-2 (fibroglycan) | Membrane spanning proteoglycan |
| (HS.1501) | | KI: increase GAG production |
| | | KO: decrease GAG production |
| SDC-3 | Syndecan-3 (N-syndecan) | Membrane spanning proteoglycan |
| (HS.158287) | | KI: increase GAG production |
| | | KO: decrease GAG production |
| SDC-4 | Syndecan-4 (ryudocan, | Membrane spanning proteoglycan |
| (HS.252189) | amphiglycan) | KI: increase GAG production |
| | | KO: decrease GAG production |
| GPC-1 | Glypican-1 | GPI-anchored membrane proteoglycan |
| (HS.328232) | | KI: increase GAG production |
| | | KO: decrease GAG production |
| GPC-2 | Glypican-2 (cerebroglycan) | GPI-anchored membrane proteoglycan |
| (HS.211701) | | KI: increase GAG production |
| | | KO: decrease GAG production |
| GPC-3 | Glypican-3 | GPI-anchored membrane proteoglycan |
| (HS.119651) | | KI: increase GAG production |
| | | KO: decrease GAG production |
| GPC-4 | Glypican-4 | GPI-anchored membrane proteoglycan |
| (HS.58367) | | KI: increase GAG production |
| | | KO: decrease GAG production |
| GPC-5 | Glypican-5 | GPI-anchored membrane proteoglycan |
| (HS.76828) | | KI: increase GAG production |
| | | KO: decrease GAG production |
| GPC-6 | Glypican-6 | GPI-anchored membrane proteoglycan |
| (HS.118407) | | KI: increase GAG production |
| | | KO: decrease GAG production |
| CD44 | CD44 (epican) | Membrane spanning proteoglycan |
| (HS.502328) | | KI: increase GAG production |
| | | KO: decrease GAG production |
| HSPG2 | Perlecan (HSPG2) | Secreted basement membrane proteoglycan |
| (HS.211573) | | KI: increase GAG production |
| | | KO: decrease GAG production |
| AGRN | Agrin | Secreted basement membrane proteoglycan |
| (HS. 273330) | | KI: increase GAG production |
| | | KO: decrease GAG production |
| SGCN | Serglycin | Intracellular granule proteoglycan |
| (HS.451015) | | KI: increase GAG production |
| | | KO: decrease GAG production |

Additional steps contemplated in methods herein include treating the genetically modified cells to degranulate the cell line. Multiple suitable methods of degranulating cells include but are not limited to methods selected from the group consisting of Antigen -IgE induced FcεRI aggregation on the degranulating cell surface, contacting the cell line to a degranulating agent, altering the culture temperature, altering the culture medium pH, altering the culture medium salt concentration, and agitation. Suitable degranulating agents include but are not limited to calcium ionophore A23187, compound 48/80, tetradecanoyl phorbol acetate (TPA), and substance P.

### Methods of Culturing Stem Cells

Also provided herein are methods of culturing stem cells, comprising culturing the stem cells in a media comprising heparin or heparan sulfate that has been produced in methods disclosed herein. In some cases, methods herein comprise contacting a stem cell to a composition comprising a heparin or a heparan sulfate isolated from a genetically modified cell line. Suitable stem cells for culturing with media comprising heparin and/or heparan sulfate produced using methods herein include but are not limited to hematopoietic stem cells, mesenchymal stem cells, neural stem cells, epithelial stem cells, skin stem cells, embryonic stem cells, and induced pluripotent stem cells.

Heparin and heparan sulfate produced by methods disclosed herein comprises culturing genetically modified cell lines such as at least one of a mastocytoma cell line and a basophil neoplastic cell line. Non-limiting exemplary mastocytoma cell lines for methods herein include MST cells, P815 cells, MC/9 cells, SI/SI4 cells, 10P2 cells, 11P0-1 cells, and 10P12 cells. In some embodiments, the genetically modified cell line is RT4 cells, 682B cells, 751G cells, 1016T cells, KK-47, MGH-U1, MHG-U2, MGH-U3, or MGH-U4. In some cases, the genetically modified cell line is deficient for one or more of chondroitin sulfate synthase 1 (CHSY1), chondroitin sulfate N-acetylgalactosaminyltransferase 2 (CSGALNACT2), chondroitin sulfate N-acetylgalactosaminyltransferase 1 (CSGALNACT1), Heparan sulfate 2-O-sulfotransferase (HS2ST), and heparan sulfate C5-epimerase (GLCE). Alternately or in addition, the genetically modified cell line is deficient for a heparan sulfate catabolic enzyme. Heparan sulfate catabolic enzyme are contemplated to comprise one or more of heparanase (HPSE), beta-glucoronidase (GUSB), sulfamidase (SGSH), heparan alpha-glucosaminide N-acetyltransferase (HGNAT), alpha-N-acetyl glucosaminidase (GSNAT), uronate-2-O-sulfatases (IDS and GDS), alpha-L-iduronidase (IDUA), and heparan sulfate 6-O-endosulfatase (SULF1-2). In further embodiments, the genetically modified cell line overexpresses one or more of heparanase, a protease, a polymerase, a sulfotransferase, an endosulfatase, and a proteoglycan protein core. Proteases for methods herein include one or more of matrix metalloproteinase (MMP), MMP2, MMP3, MMP7, MMP9, MT1-MMP, MT3-MMP, ADAM17, ADAMTS1, ADAMTS4, trypsin, and chymotrypsin. Suitable proteoglycan protein cores include one or more of serglycin, a syndecan, a glypican, CD44 isoforms (CD44E), perlecan, collagen XVIII, and agrin, and recombinant versions thereof. In some cases, the serglycin comprises a human serglycin, a pig serglycin, a mouse serglycin. Modifications to serglycan include modification of the number of GAG attachment sites in the serglycin. In some cases, the syndecan is selected from the group consisting of syndecan-1, syndecan-2, syndecan-3, syndecan-4, and syndecan ectodomains. Exemplary glypicans are selected from the group consisting of glypican 1, glypican 2, glypican 3, glypican 4, glypican 5, and glypican 6 and ectodomains.

### Methods of Treatment

Therefore, disclosed herein are methods of treating diseases and conditions in subjects in need thereof by administering an effective amount of one or more heparin and/or heparan sulfate purified from mastocytoma or basophil neoplastic cell lines by methods provided herein. Mastocytoma cell lines are contemplated to include MST cells, P815 cells, MC/9 cells, SI/SI4 cells, 10P2 cells, 11P0-1 cells, and 10P12 cells. In some embodiments, the genetically modified cell line is RT4 cells, 682B cells, 751G cells, 1016T cells, KK-47, MGH-U1, MHG-U2, MGH-U3, or MGH-U4. In some embodiments, the disease or condition comprises one or more of wounds, bone fractures, skin conditions, cancers, angiogenesis, inflammatory diseases, neurological diseases, and other suitable diseases.

In some embodiments, the compositions or pharmaceutical compositions disclosed herein are administered to the subject by any suitable route, found to be effective in treating thrombosis, inflammation, cancer, microbial infections, neurodegenerative disorders and wound healing among others. In some embodiments, the compositions or pharmaceutical compositions disclosed herein are administered orally, rectally, sublingually, sublabially, buccally, epidurally, entracerebrally, intracerebroventricalarly, topically, transdermally, nasally, intraarterially, intraarticularly, intracardiacally, intradermally, subcutaneously, intralesionally, intramuscular, intraocularly, intraosseously, intraperitoneally, intrathecally, intravenously, transmucosally, or any other route of administration known by one of skill in the art.

Methods of treatment herein comprise administering an effective amount of a composition comprising one or more heparin and/or heparan sulfate preparations provided herein to a subject in need thereof. In some cases, the method comprises identifying a patient in need of treatment. In some cases, the method comprises monitoring the patient for an improvement in one or more symptoms. In some cases, the method comprises administration of one, two three, four, five, six, seven, eight, nine, ten, or more additional doses of the composition until the subject experiences improvement in one or more symptoms. In some cases, the method comprises continuous or chronic administration of the composition to improve one or more symptoms.

### Methods of Treating Thrombosis

In some embodiments, there is provided a method of treating thrombosis in a subject in need thereof comprising administering to the subject an effective amount of one or more heparin, and/or heparan sulfate prepared from genetically modified mastocytoma cells (e.g. MST cells) or basophil neoplastic cells via methods provided herein. In some embodiments, the thrombosis comprises, venous thrombosis, deep vein thrombosis, portal vein thrombosis, renal vein thrombosis, jugular vein thrombosis, Budd-Chiari syndrome, Paget-Schroetter disease, Cerebral venous sinus thrombosis, Cavernous sinus thrombosis, arterial thrombosis, stroke, myocardial infarction, Hepatic artery thrombosis, acute coronary syndrome atrial fibrillation, or pulmonary embolism. In some embodiments, treatment of the thrombosis reduces swelling, pain, tenderness, skin discoloration, shortness of breath, chest pain, rapid heart rate, cough, or other symptom of thrombosis. In some embodiments, the method prevents or eliminates a blood clot. In some embodiments, the method prevents or eliminates a blood clot without causing heparin-induced thrombocytopenia.

In methods provided herein, treatment of thrombosis with heparin or heparan sulfate compositions produced using methods herein have reduced risk of complications and side effects that often result with treatment using unfractionated heparin. In some cases, heparin or heparan sulfate compositions produced using methods herein useful in methods of treating thrombosis herein have reduced binding to platelet factor 4 (PF4). In some cases, the composition is purified from a mastocytoma (MST) cell line or a basophil neoplastic cell line genetically modified to be deficient for heparan sulfate 2-O-sulfotransferase (HS2ST). In some cases, the composition is purified from a MST cell line or a basophil neoplastic cell line genetically modified to overexpress heparan sulfate-6-O-endosulfatase 1 or 2 (SULF1-2).

Methods of treating thrombosis in a subject in need thereof provided herein comprise administering a composition comprising a heparin or a heparan sulfate isolated from a genetically modified cell line. In some cases, the genetically modified cell line comprises at least one of a mastocytoma (MST) cell line or a basophil neoplastic cell line. In some cases, the genetically modified cell line is deficient for one or more of chondroitin sulfate synthase 1 (CHSY1), chondroitin sulfate N-acetylgalactosaminyltransferase 2 (CSGALNACT2), chondroitin sulfate N-acetylgalactosaminyltransferase 1 (CSGALNACT1), Heparan sulfate 2-O-sulfotransferase (HS2ST), and heparan sulfate C5-epimerase (GLCE). In some cases, the genetically modified cell line is deficient for a heparan sulfate catabolic enzyme. In some cases, the heparan sulfate catabolic enzyme comprises one or more of heparanase (HPSE), beta-glucoronidase (GUSB), sulfamidase (SGSH), heparan alpha-glucosaminide N-acetyltransferase (HGNAT), alpha-N-acetyl glucosaminidase (GSNAT), uronate-2-O-sulfatases (IDS and GDS), alpha-L-iduronidase (IDUA), and heparan sulfate 6-O-endosulfatase (SULF1-2). In some cases, the genetically modified MST cell line overexpresses one or more of heparanase, a protease, a polymerase, a sulfotransferase, an endosulfatase, and a proteoglycan protein core. In some cases, the protease comprises one or more of matrix metalloproteinase (MMP), MMP2, MMP3, MMP7, MMP9, MT1-MMP, MT3-MMP, ADAM17, ADAMTS1, ADAMTS4, trypsin, and chymotrypsin. In some cases, the proteoglycan protein core comprises one or more of serglycin, a syndecan, a glypican, perlecan, and CD44 core protein (CD44E). In some cases, the serglycin comprises a human serglycin, a pig serglycin, a mouse serglycin. In some cases, the number of GAG attachment sites in the serglycin is modified. In some cases, the syndecan is selected from the group consisting of syndecan-1, syndecan-2, syndecan-3, syndecan-4, and syndecan ectodomains. In some cases, the glypican is selected from the group consisting of glypican 1, glypican 2, glypican 3, glypican 4, glypican 5, and glypican 6 and ectodomains.

### Methods of Treating Wounds

In some embodiments, there is provided a method of treating a wound in a subject in need thereof comprising administering to the subject an effective amount of heparin, and/or heparan sulfate prepared from genetically modified mastocytoma cells (e.g. MST cells) or basophil neoplastic cells via methods provided herein. In some embodiments, the wound comprises an incision, a laceration, an abrasion, an avulsion, a puncture wound, a penetration wound, a gunshot wound, a hematoma, or a crush injury. In some embodiments, the method reduces symptoms or complications related to a wound, such as drainage, pus, fever, or lymph node swelling. In some embodiments, the method speeds the healing time of a wound. In some embodiments, the method treats diabetic wounds. In some embodiments, the method treats a nerve injury. In some embodiments, the method treats a spinal cord injury.

Methods of treating wounds in a subject in need thereof provided herein comprise administering a composition comprising a heparin or a heparan sulfate isolated from a genetically modified cell line. In some cases, the genetically modified cell line comprises at least one of a mastocytoma (MST) cell line and a basophil neoplastic cell line. In some cases, the genetically modified cell line is deficient for one or more of chondroitin sulfate synthase 1 (CHSY1), chondroitin sulfate N-acetylgalactosaminyltransferase 2 (CSGALNACT2), chondroitin sulfate N-acetylgalactosaminyltransferase 1 (CSGALNACT1), Heparan sulfate 2-O-sulfotransferase (HS2ST), and heparan sulfate C5-epimerase (GLCE). In some cases, the genetically modified cell line is deficient for a heparan sulfate catabolic enzyme. In some cases, the heparan sulfate catabolic enzyme comprises one or more of heparanase (HPSE), beta-glucoronidase (GUSB), sulfamidase (SGSH), heparan alpha-glucosaminide N-acetyltransferase (HGNAT), alpha-N-acetyl glucosaminidase (GSNAT), uronate-2-O-sulfatases (IDS and GDS), alpha-L-iduronidase (IDUA), and heparan sulfate 6-O-endosulfatase (SULF1-2). In some cases, the genetically modified MST cell line overexpresses one or more of heparanase, a protease, a polymerase, a sulfotransferase, an endosulfatase, and a proteoglycan protein core. In some cases, the protease comprises one or more of matrix metalloproteinase (MMP), MMP2, MMP3, MMP7, MMP9, MT1-MMP, MT3-MMP, ADAM17, ADAMTS1, ADAMTS4, trypsin, and chymotrypsin. In some cases, the proteoglycan protein core comprises one or more of serglycin, a syndecan, a glypican, perlecan, and CD44 core protein (CD44E). In some cases, the serglycin comprises a human serglycin, a pig serglycin, a mouse serglycin. In some cases, the number of GAG attachment sites in the serglycin is modified. In some cases, the syndecan is selected from the group consisting of syndecan-1, syndecan-2, syndecan-3, syndecan-4, and syndecan ectodomains. In some cases, the glypican is selected from the group consisting of glypican 1, glypican 2, glypican 3, glypican 4, glypican 5, and glypican 6 and ectodomains.

### Treatment of Inflammation

In some embodiments, there is provided a method of treating inflammation in a subject in need thereof comprising administering to the subject an effective amount of heparin, and/or heparan sulfate prepared from genetically modified mastocytoma cells (e.g. MST cells) or basophil neoplastic cells via methods provided herein. Inflammation herein is contemplated to comprise inflammatory diseases often caused by aberrant immune responses, such as autoimmunity and other diseases associated with an over-active immune response or inappropriate immune response. In some embodiments, the inflammation comprises rheumatoid arthritis, juvenile rheumatoid arthritis, osteoarthritis, psoriatic arthritis, multiple sclerosis (MS), encephalomyelitis, myasthenia gravis, systemic lupus erythematosus (SLE), asthma, allergic asthma, autoimmune thyroiditis, atopic dermatitis, eczematous dermatitis, psoriasis, Sjögren's Syndrome, Crohn's disease, aphthous ulcer, iritis, conjunctivitis, keratoconjunctivitis, ulcerative colitis (UC), inflammatory bowel disease (IBD), cutaneous lupus erythematosus, scleroderma, vaginitis, proctitis, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, interstitial lung fibrosis, chronic obstructive pulmonary disease (COPD), cystic fibrosis, alpha-1 antitrypsin deficiency, acute respiratory distress syndrome (ARDS), Hashimoto's thyroiditis, autoimmune polyglandular syndrome, insulin-dependent diabetes mellitus (IDDM, type I diabetes), insulin-resistant diabetes mellitus (type 2 diabetes), immune-mediated infertility, autoimmune Addison's disease, pemphigus vulgaris, pemphigus foliaceus, dermatitis herpetiformis, autoimmune alopecia, vitiligo, autoimmune hemolytic anemia, autoimmune thrombocytopenic purpura, pernicious anemia, Guillain-Barre syndrome, stiff-man syndrome, acute rheumatic fever, sympathetic ophthalmia, Goodpasture's syndrome, systemic necrotizing vasculitis, antiphospholipid syndrome or an allergy, Behcet's disease, X-linked lymphoproliferative syndrome (SH2D1A/SAP deficiency), hyper IgE syndrome or Graft vs. Host Disease (GVHD). In some embodiments, treatment of the inflammation reduces pain, redness, swelling, loss of joint function, fever, chills, fatigue, headache, loss of appetite, muscle stiffness, or other symptom associated with inflammation or inflammatory disease.

Methods of treating inflammation in a subject in need thereof provided herein comprise administering a composition comprising a heparin or a heparan sulfate isolated from a genetically modified cell line. In some cases, the genetically modified cell line comprises at least one of a mastocytoma (MST) cell line and a basophil neoplastic cell line. In some cases, the genetically modified cell line is deficient for one or more of chondroitin sulfate synthase 1 (CHSY1), chondroitin sulfate N-acetylgalactosaminyltransferase 2 (CSGALNACT2), chondroitin sulfate N-acetylgalactosaminyltransferase 1 (CSGALNACT1), Heparan sulfate 2-O-sulfotransferase (HS2ST), and heparan sulfate C5-epimerase (GLCE). In some cases, the genetically modified cell line is deficient for a heparan sulfate catabolic enzyme. In some cases, the heparan sulfate catabolic enzyme comprises one or more of heparanase (HPSE), beta-glucoronidase (GUSB), sulfamidase (SGSH), heparan alpha-glucosaminide N-acetyltransferase (HGNAT), alpha-N-acetyl glucosaminidase (GSNAT), uronate-2-O-sulfatases (IDS and GDS), alpha-L-iduronidase (IDUA), and heparan sulfate 6-O-endosulfatase (SULF1-2). In some cases, the genetically modified MST cell line overexpresses one or more of heparanase, a protease, a polymerase, a sulfotransferase, an endosulfatase, and a proteoglycan protein core. In some cases, the sulfotransferase is heparan sulfate 2-O-sulfotransferase. In some cases, the protease comprises one or more of matrix metalloproteinase (MMP), MMP2, MMP3, MMP7, MMP9, MT1-MMP, MT3-MMP, ADAM17, ADAMTS1, ADAMTS4, trypsin, and chymotrypsin. In some cases, the proteoglycan protein core comprises one or more of serglycin, a syndecan, a glypican, perlecan, and CD44 core protein (CD44E). In some cases, the serglycin comprises a human serglycin, a pig serglycin, a mouse serglycin. In some cases, the number of GAG attachment sites in the serglycin is modified. In some cases, the syndecan is selected from the group consisting of syndecan-1, syndecan-2, syndecan-3, syndecan-4, and syndecan ectodomains. In some cases, the glypican is selected from the group consisting of glypican 1, glypican 2, glypican 3, glypican 4, glypican 5, and glypican 6 and ectodomains.

### Treatment of Lung Inflammation

In some embodiments, there is provided a method of treating lung inflammation in a subject in need thereof comprising administering to the subject an effective amount of heparin, and/or heparan sulfate prepared from genetically modified mastocytoma cells (e.g. MST cells) or basophil neoplastic cells via methods provided herein. Lung inflammation herein is contemplated to comprise inflammatory diseases of the lung often caused by aberrant immune responses, such as autoimmunity and other diseases associated with an over-active immune response or inappropriate immune response. In some embodiments, the lung inflammation comprises asthma, allergic asthma, interstitial lung fibrosis, chronic obstructive pulmonary disease (COPD), cystic fibrosis, alpha-1 antitrypsin deficiency, or acute respiratory distress syndrome (ARDS). In some embodiments, treatment of the inflammation improves breathing and blood oxygenation, reduces incidence of shortness of breath, wheezing, chest tightness, cough, and respiratory infections. In some cases, the treatment reduces need for supplemental oxygen, use of rescue inhalers or steroid medication.

Methods of treating inflammation in a subject in need thereof provided herein comprise administering a composition comprising a heparin or a heparan sulfate isolated from a genetically modified cell line. In some cases, the genetically modified cell line comprises at least one of a mastocytoma (MST) cell line and a basophil neoplastic cell line. In some cases, the genetically modified cell line is deficient for one or more of chondroitin sulfate synthase 1 (CHSY1), chondroitin sulfate N-acetylgalactosaminyltransferase 2 (CSGALNACT2), chondroitin sulfate N-acetylgalactosaminyltransferase 1 (CSGALNACT1), and heparan sulfate C5-epimerase (GLCE). In some cases, the genetically modified cell line is deficient for a heparan sulfate catabolic enzyme. In some cases, the heparan sulfate catabolic enzyme comprises one or more of heparanase (HPSE), beta-glucoronidase (GUSB), sulfamidase (SGSH), heparan alpha-glucosaminide N-acetyltransferase (HGNAT), alpha-N-acetyl glucosaminidase (GSNAT), uronate-2-O-sulfatases (IDS and GDS), alpha-L-iduronidase (IDUA), and heparan sulfate 6-O-endosulfatase (SULF1-2). In some cases, the genetically modified MST cell line overexpresses one or more of heparanase, a protease, a polymerase, a sulfotransferase, an endosulfatase, and a proteoglycan protein core. In some cases, the sulfotransferase is heparan sulfate 2-O-sulfotransferase. In some cases, the protease comprises one or more of matrix metalloproteinase (MMP), MMP2, MMP3, MMP7, MMP9, MT1-MMP, MT3-MMP, ADAM17, ADAMTS1, ADAMTS4, trypsin, and chymotrypsin. In some cases, the proteoglycan protein core comprises one or more of serglycin, a syndecan, a glypican, perlecan, and CD44 core protein (CD44E). In some cases, the serglycin comprises a human serglycin, a pig serglycin, a mouse serglycin. In some cases, the number of GAG attachment sites in the serglycin is modified. In some cases, the syndecan is selected from the group consisting of syndecan-1, syndecan-2, syndecan-3, syndecan-4, and syndecan ectodomains. In some cases, the glypican is selected from the group consisting of glypican 1, glypican 2, glypican 3, glypican 4, glypican 5, and glypican 6 and ectodomains.

### Treatment of Cancer

In some embodiments, there is provided a method of treating cancer in a subject in need thereof comprising administering to the subject an effective amount of heparin, and/or heparan sulfate prepared from genetically modified mastocytoma cells (e.g. MST cells) or basophil neoplastic cells via methods provided herein. In some embodiments, the cancer comprises Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, or other type of cancer. In some embodiments, the cancer comprises a metastasis of one or more of the above cancers.

Methods of treating cancer in a subject in need thereof provided herein comprise administering a composition comprising a heparin or a heparan sulfate isolated from a genetically modified cell line. In some cases, the genetically modified cell line comprises at least one of a mastocytoma (MST) cell line and a basophil neoplastic cell line. In some cases, the genetically modified cell line is deficient for one or more of chondroitin sulfate synthase 1 (CHSY1), chondroitin sulfate N-acetylgalactosaminyltransferase 2 (CSGALNACT2), chondroitin sulfate N-acetylgalactosaminyltransferase 1 (CSGALNACT1), Heparan sulfate 2-O-sulfotransferase (HS2ST), and heparan sulfate C5-epimerase (GLCE). In some cases, the genetically modified cell line is deficient for a heparan sulfate catabolic enzyme. In some cases, the heparan sulfate catabolic enzyme comprises one or more of heparanase (HPSE), beta-glucoronidase (GUSB), sulfamidase (SGSH), heparan alpha-glucosaminide N-acetyltransferase (HGNAT), alpha-N-acetyl glucosaminidase (GSNAT), uronate-2-O-sulfatases (IDS and GDS), alpha-L-iduronidase (IDUA), and heparan sulfate 6-O-endosulfatase (SULF1-2). In some cases, the genetically modified MST cell line overexpresses one or more of heparanase, a protease, a polymerase, a sulfotransferase, an endosulfatase, and a proteoglycan protein core. In some cases, the protease comprises one or more of matrix metalloproteinase (MMP), MMP2, MMP3, MMP7, MMP9, MT1-MMP, MT3-MMP, ADAM17, ADAMTS1, ADAMTS4, trypsin, and chymotrypsin. In some cases, the proteoglycan protein core comprises one or more of serglycin, a syndecan, a glypican, perlecan, and CD44 core protein (CD44E). In some cases, the serglycin comprises a human serglycin, a pig serglycin, a mouse serglycin. In some cases, the number of GAG attachment sites in the serglycin is modified. In some cases, the syndecan is selected from the group consisting of syndecan-1, syndecan-2, syndecan-3, syndecan-4, and syndecan ectodomains. In some cases, the glypican is selected from the group consisting of glypican 1, glypican 2, glypican 3, glypican 4, glypican 5, and glypican 6 and ectodomains.

Efficacy in treating cancer in particular may be measured by any suitable metric. In some embodiments, therapeutic efficacy is measured based on an effect of treating a proliferative disorder, such as cancer. In general, therapeutic efficacy of the methods and compositions of the invention, with regard to the treatment of a proliferative disorder (e.g. cancer, whether benign or malignant), may be measured by the degree to which the methods and compositions promote inhibition of tumor cell proliferation, the inhibition of tumor vascularization, the eradication of tumor cells, and/or a reduction in the size of at least one tumor such that a human is treated for the proliferative disorder. Several parameters to be considered in the determination of therapeutic efficacy are discussed herein. The proper combination of parameters for a particular situation can be established by the clinician. The progress of the inventive method in treating cancer (e.g., reducing tumor size or eradicating cancerous cells) can be ascertained using any suitable method, such as those methods currently used in the clinic to track tumor size and cancer progress. In some embodiments, the primary efficacy parameter used to evaluate the treatment of cancer preferably is a reduction in the size of a tumor. Tumor size can be determined using any suitable technique, such as measurement of dimensions, or estimation of tumor volume using available computer software, such as FreeFlight software developed at Wake Forest University that enables accurate estimation of tumor volume. Tumor size can be determined by tumor visualization using, for example, CT, ultrasound, SPECT, spiral CT, MRI, photographs, and the like. In embodiments where a tumor is surgically resected after completion of the therapeutic period, the presence of tumor tissue and tumor size can be determined by gross analysis of the tissue to be resected, and/or by pathological analysis of the resected tissue.

Desirably, the growth of a tumor is stabilized (i.e., one or more tumors do not increase more than 1%, 5%, 10%, 15%, or 20% in size, and/or do not metastasize) as a result of treatment. In some embodiments, a tumor is stabilized for at least about 1, 2, 3, 4, 5, 6, 7, 8, 9, 10, 11, 12, or more weeks. In some embodiments, a tumor is stabilized for at least about 1, 2, 3, 4, 5, 6, 7, 8, 9, 10, 11, 12, or more months. In some embodiments, a tumor is stabilized for at least about 1, 2, 3, 4, 5, 6, 7, 8, 9, 10, or more years. In some embodiments, the size of a tumor is reduced at least about 5% (e.g., at least about 10%, 15%, 20%, or 25%). In some embodiments, tumor size is reduced at least about 30% (e.g., at least about 35%, 40%, 45%, 50%, 55%, 60%, or 65%). In some embodiments, tumor size is reduced at least about 70% (e.g., at least about 75%, 80%, 85%, 90%, or 95%). In some embodiments, the tumor is completely eliminated, or reduced below a level of detection. In some embodiments, a subject remains tumor free (e.g. in remission) for at least about 1, 2, 3, 4, 5, 6, 7, 8, 9, 10, 11, 12, or more weeks following treatment. In some embodiments, a subject remains tumor free for at least about 1, 2, 3, 4, 5, 6, 7, 8, 9, 10, 11, 12, or more months following treatment. In some embodiments, a subject remains tumor free for at least about 1, 2, 3, 4, 5, 6, 7, 8, 9, 10, or more years after treatment.

When a tumor is subject to surgical resection following completion of the therapeutic period, the efficacy of the inventive method in reducing tumor size can be determined by measuring the percentage of resected tissue that is necrotic (i.e., dead). In some embodiments, a treatment is therapeutically effective if the necrosis percentage of the resected tissue is greater than about 20% (e.g., at least about 30%, 40%, 50%, 60%, 70%, 80%, 90%, or 100%), more preferably about 90% or greater (e.g., about 90%, 95%, or 100%). Most preferably, the necrosis percentage of the resected tissue is about 100%, that is, no tumor tissue is present or detectable.

A number of secondary parameters can be employed to determine the efficacy of the inventive method. Examples of secondary parameters include, but are not limited to, detection of new tumors, detection of tumor antigens or markers (e.g., CEA, PSA, or CA-125), biopsy, surgical downstaging (i.e., conversion of the surgical stage of a tumor from unresectable to resectable), PET scans, survival, disease progression-free survival, time to disease progression, quality of life assessments such as the Clinical Benefit Response Assessment, and the like, all of which can point to the overall progression (or regression) of cancer in a human. Biopsy is particularly useful in detecting the eradication of cancerous cells within a tissue. Radioimmunodetection (RAID) is used to locate and stage tumors using serum levels of markers (antigens) produced by and/or associated with tumors (“tumor markers” or “tumor-associated antigens”), and can be useful as a pre-treatment diagnostic predicate, a post-treatment diagnostic indicator of recurrence, and a post-treatment indicator of therapeutic efficacy. Examples of tumor markers or tumor-associated antigens that can be evaluated as indicators of therapeutic efficacy include, but are not limited to, carcinembryonic antigen (CEA) prostate-specific antigen (PSA), CA-125, CA19-9, ganglioside molecules (e.g., GM2, GD2, and GD3), MART-1, heat shock proteins (e.g., gp96), sialyl Tn (STn), tyrosinase, MUC-1, HER-2/neu, c-erb-B2, KSA, PSMA, p53, RAS, EGF-R, VEGF, MAGE, and gp100. Other tumor-associated antigens are known in the art. RAID technology in combination with endoscopic detection systems also efficiently distinguishes small tumors from surrounding tissue.

In some embodiments, the treatment of cancer in a human patient is evidenced by one or more of the following results: (a) the complete disappearance of a tumor (i.e., a complete response), (b) about a 25% to about a 50% reduction in the size of a tumor for at least four weeks after completion of the therapeutic period as compared to the size of the tumor before treatment, (c) at least about a 50% reduction in the size of a tumor for at least four weeks after completion of the therapeutic period as compared to the size of the tumor before the therapeutic period, and (d) at least a 2% decrease (e.g., about a 5%, 10%, 20%, 30%, 40%, 50%, 60%, 70%, 80% or 90% decrease) in a specific tumor-associated antigen level at about 4-12 weeks after completion of the therapeutic period as compared to the tumor-associated antigen level before the therapeutic period. While at least a 2% decrease in a tumor-associated antigen level is preferred, any decrease in the tumor-associated antigen level is evidence of treatment of a cancer in a patient. For example, with respect to unresectable, locally advanced pancreatic cancer, treatment can be evidenced by at least a 10% decrease in the CA19-9 tumor-associated antigen level at 4-12 weeks after completion of the therapeutic period as compared to the CA19-9 level before the therapeutic period. Similarly, with respect to locally advanced rectal cancer, treatment can be evidenced by at least a 10% decrease in the CEA tumor-associated antigen level at 4-12 weeks after completion of the therapeutic period as compared to the CEA level before the therapeutic period.

With respect to quality of life assessments, such as the Clinical Benefit Response Criteria, the therapeutic benefit of the treatment in accordance with the invention can be evidenced in terms of pain intensity, analgesic consumption, and/or the Karnofsky Performance Scale score. The Karnofsky Performance Scale allows patients to be classified according to their functional impairment. The Karnofsky Performance Scale is scored from 0-100. In general, a lower Karnofsky score is predictive of a poor prognosis for survival. Thus, the treatment of cancer in a human patient alternatively, or in addition, is evidenced by (a) at least a 50% decrease (e.g., at least a 60%, 70%, 80%, 90%, or 100% decrease) in pain intensity reported by a patient, such as for any consecutive four week period in the 12 weeks after completion of treatment, as compared to the pain intensity reported by the patient before treatment, (b) at least a 50% decrease (e.g., at least a 60%, 70%, 80%, 90%, or 100% decrease) in analgesic consumption reported by a patient, such as for any consecutive four week period in the 12 weeks after completion of treatment as compared to the analgesic consumption reported by the patient before treatment, and/or (c) at least a 20 point increase (e.g., at least a 30 point, 50 point, 70 point, or 90 point increase) in the Karnofsky Performance Scale score reported by a patient, such as for any consecutive four week period in the 12 weeks after completion of the therapeutic period as compared to the Karnofsky Performance Scale score reported by the patient before the therapeutic period.

The treatment of a proliferative disorder (e.g. cancer, whether benign or malignant) in a human patient desirably is evidenced by one or more (in any combination) of the foregoing results, although alternative or additional results of the referenced tests and/or other tests can evidence treatment efficacy.

In some embodiments, tumor size is reduced preferably without significant adverse events in the subject. Adverse events are categorized or “graded” by the Cancer Therapy Evaluation Program (CTEP) of the National Cancer Institute (NCI), with Grade 0 representing minimal adverse side effects and Grade 4 representing the most severe adverse events. The NCI toxicity scale (published April 1999) and Common Toxicity Criteria Manual (updated August 1999) is available through the NCI, e.g., or in the Investigator's Handbook for participants in clinical trials of investigational agents sponsored by the Division of Cancer Treatment and Diagnosis, NCI (updated March 1998). Desirably, methods described herein are associated with minimal adverse events, e.g. Grade 0, Grade 1, or Grade 2 adverse events, as graded by the CTEP/NCI. However, reduction of tumor size, although preferred, is not required in that the actual size of tumor may not shrink despite the eradication (such as in necrosis) of tumor cells. Eradication of cancerous cells is sufficient to realize a therapeutic effect. Likewise, any reduction in tumor size is sufficient to realize a therapeutic effect.

Detection, monitoring, and rating of various cancers in a human are further described in Cancer Facts and Figures 2001, American Cancer Society, New York, N.Y. Accordingly, a clinician can use standard tests to determine the efficacy of the various embodiments of the inventive method in treating cancer. However, in addition to tumor size and spread, the clinician also may consider quality of life and survival of the patient in evaluating efficacy of treatment.

### Treatment of Neurodegenerative Disorders

In some embodiments, there is provided a method of treating a neurodegenerative disorder in a subject in need thereof comprising administering to the subject an effective amount of heparin, and/or heparan sulfate prepared from genetically modified mastocytoma cells (e.g. MST cells) or basophil neoplastic cells via methods provided herein. In some embodiments, the neurodegenerative disorder comprises Alzheimer's disease, Parkinson's disease, Huntington's disease, Amyotrophic lateral sclerosis, Dementia, Transmissible spongiform encephalopathy, Dentatorubro-pallidoluysian atrophy, Spinal and bulbar muscular atrophy, Spinocerebellar ataxia Type 1, Spinocerebellar ataxia Type 2, Spinocerebellar ataxia Type 3, Spinocerebellar ataxia Type 6, Spinocerebellar ataxia Type 7, or Spinocerebellar ataxia Type 17. In some embodiments, the method reduces symptoms of a neurodegenerative disorder such as memory loss, disorientation, confusion, mood and/or personality disorder, tremor, bradykinesia, muscle rigidity, balance impairment, speech disorder, choria, dystonia, ataxia, swallowing disorder, irritability, sadness, apathy, social withdrawal, insomnia, fatigue, suicidal thoughts, weakness, speech disorder, muscle cramping, impaired coordination, stumbling, unsteady gait, uncontrolled movements, slurred speech, vocal changes, or headache. In some embodiments, the method delays onset of more severe symptoms. In some embodiments, the delay is 1, 2, 3, 4, 5, 6 or more weeks, 1, 2, 3, 4, 5, 6, 7, 8, 9, 10, 11, 12, or more months, 1, 2, 3, 4, 5, 6, 7, 8, 9, 10, 11, 12, 13, 14, 15, 20, 25, 30, or more years.

Methods of treating neurodegenerative disorders in a subject in need thereof provided herein comprise administering a composition comprising a heparin or a heparan sulfate isolated from a genetically modified cell line. In some cases, the genetically modified cell line comprises at least one of a mastocytoma (MST) cell line and a basophil neoplastic cell line. In some cases, the genetically modified cell line is deficient for one or more of chondroitin sulfate synthase 1 (CHSY1), chondroitin sulfate N-acetylgalactosaminyltransferase 2 (CSGALNACT2), chondroitin sulfate N-acetylgalactosaminyltransferase 1 (CSGALNACT1), heparan sulfate 2-O-sulfotransferase (HS2ST), and heparan sulfate C5-epimerase (GLCE). In some cases, the genetically modified cell line is deficient for a heparan sulfate catabolic enzyme. In some cases, the heparan sulfate catabolic enzyme comprises one or more of heparanase (HPSE), beta-glucoronidase (GUSB), sulfamidase (SGSH), heparan alpha-glucosaminide N-acetyltransferase (HGNAT), alpha-N-acetyl glucosaminidase (GSNAT), uronate-2-O-sulfatases (IDS and GDS), alpha-L-iduronidase (IDUA), and heparan sulfate 6-O-endosulfatase (SULF1-2). In some cases, the genetically modified MST cell line overexpresses one or more of heparanase, a protease, a polymerase, a sulfotransferase, an endosulfatase, and a proteoglycan protein core. In some cases, the protease comprises one or more of matrix metalloproteinase (MMP), MMP2, MMP3, MMP7, MMP9, MT1-MMP, MT3-MMP, ADAM17, ADAMTS1, ADAMTS4, trypsin, and chymotrypsin. In some cases, the proteoglycan protein core comprises one or more of serglycin, a syndecan, a glypican, perlecan, and CD44 core protein (CD44E). In some cases, the serglycin comprises a human serglycin, a pig serglycin, a mouse serglycin. In some cases, the number of GAG attachment sites in the serglycin is modified. In some cases, the syndecan is selected from the group consisting of syndecan-1, syndecan-2, syndecan-3, syndecan-4, and syndecan ectodomains. In some cases, the glypican is selected from the group consisting of glypican 1, glypican 2, glypican 3, glypican 4, glypican 5, and glypican 6 and ectodomains.

### Methods of Treating Bone Fractures

In some embodiments, there is provided a method of treating a bone fracture in a subject in need thereof comprising administering to the subject an effective amount of heparin, and/or heparan sulfate prepared from genetically modified mastocytoma cells (e.g. MST cells) or basophil neoplastic cells via methods provided herein. Bone fractures suitable for treatment via methods provided herein include but are not limited to stress fractures, pathologic fractures, oblique fractures, transverse fractures, comminuted fractures, greenstick fractures, buckle fractures, growth plate fractures, and other suitable fractures. In some cases, treatment of bone fractures herein speeds healing of fractures by at least about 1 week, 2 weeks, 3, weeks, 4 weeks, 5 weeks, or more. In some cases, treatment of bone fractures herein eliminates need for surgery to correct the fracture.

Methods of treating thrombosis in a subject in need thereof provided herein comprise administering a composition comprising a heparin or a heparan sulfate isolated from a genetically modified cell line. In some cases, the genetically modified cell line comprises at least one of a mastocytoma (MST) cell line and a basophil neoplastic cell line. In some cases, the genetically modified cell line is deficient for one or more of chondroitin sulfate synthase 1 (CHSY1), chondroitin sulfate N-acetylgalactosaminyltransferase 2 (CSGALNACT2), chondroitin sulfate N-acetylgalactosaminyltransferase 1 (CSGALNACT1), Heparan sulfate 2-O-sulfotransferase (HS2ST), and heparan sulfate C5-epimerase (GLCE). In some cases, the genetically modified cell line is deficient for a heparan sulfate catabolic enzyme. In some cases, the heparan sulfate catabolic enzyme comprises one or more of heparanase (HPSE), beta-glucoronidase (GUSB), sulfamidase (SGSH), heparan alpha-glucosaminide N-acetyltransferase (HGNAT), alpha-N-acetyl glucosaminidase (GSNAT), uronate-2-O-sulfatases (IDS and GDS), alpha-L-iduronidase (IDUA), and heparan sulfate 6-O-endosulfatase (SULF1-2). In some cases, the genetically modified MST cell line overexpresses one or more of heparanase, a protease, a polymerase, a sulfotransferase, an endosulfatase, and a proteoglycan protein core. In some cases, the protease comprises one or more of matrix metalloproteinase (MMP), MMP2, MMP3, MMP7, MMP9, MT1-MMP, MT3-MMP, ADAM17, ADAMTS1, ADAMTS4, trypsin, and chymotrypsin. In some cases, the proteoglycan protein core comprises one or more of serglycin, a syndecan, a glypican, perlecan, and CD44 core protein (CD44E). In some cases, the serglycin comprises a human serglycin, a pig serglycin, a mouse serglycin. In some cases, the number of GAG attachment sites in the serglycin is modified. In some cases, the syndecan is selected from the group consisting of syndecan-1, syndecan-2, syndecan-3, syndecan-4, and syndecan ectodomains. In some cases, the glypican is selected from the group consisting of glypican 1, glypican 2, glypican 3, glypican 4, glypican 5, and glypican 6 and ectodomains.

### Methods of Dermatological Treatment

In some embodiments, there is provided a method of treating a dermatological condition in a subject in need thereof comprising administering to the subject an effective amount of heparin, and/or heparan sulfate prepared from genetically modified mastocytoma cells (e.g. MST cells) or basophil neoplastic cells via methods provided herein. Dermatological conditions suitable for treatment via methods provided herein include but are not limited to a wrinkle, a pimple, a hyperpigmentation, an age-related skin condition, dryness, lack of skin elasticity, lack of skin firmness, and fine lines. In further embodiments, the skin condition is fine lines and wrinkles; age spots and dyspigmentation; skin texture, tone and elasticity; roughness and photo damage; the ability of skin to regenerate itself; environmental damage; smoothness and tightness skin; age spots, fine and coarse lines and wrinkles, fine and course periocular wrinkles, nasolabial folds, facial fine and coarse lines, skin radiance and evenness, skin firmness, hyperpigmentation, dark spots and/or patches, skin brightness and youthful appearance, photo aged skin, intrinsically and extrinsically aged skin, skin cellular turnover, skin barrier, skin's ability to retain moisture, brown and red blotchiness, redness, skin epidermal thickness, dermal epidermal junction, pore size and number of pores, or a combination thereof. In some cases, the methods rejuvenate sun damaged and aging skin; improves the appearance of fine lines and wrinkles; promotes cell renewal; diminishes the appearance of age spots and dyspigmentation; improves skin tone, texture and elasticity; reduces roughness and photo damage; prevents or reduces environmental damage; plumps the skin; brightens the skin; lightens the skin; strengthens the ability of skin to regenerate itself; improves the appearance of age spots; brightens and lightens age spots; improves skin firmness, elasticity, resiliency; smooths, tightens, or fills in fine lines on the skin; reduces the appearance of dark circles under the eye; improves lip texture or condition; enhances natural lip color; increases lip volume; promotes epithelialization of post-procedure skin; restores the skin's barrier or moisture balance; improves the appearance of age spots; improves the appearance of skin pigmentation, or a combination thereof In one embodiment, the compositions reduce the appearance of fine lines and wrinkles; diminish the appearance of age spots and dyspigmentation; improve skin texture, tone and elasticity; reduce roughness and photo damage; strengthen the ability of skin to regenerate itself; prevent or reduce environmental damage; smooth and tightens skin; brighten and lighten age spots, reduce in fine and coarse lines and wrinkles, improve appearance of fine and course periocular wrinkles, improve appearance of nasolabial folds, improve perioral wrinkles, improve facial fine and coarse lines, improve skin tone, radiance and evenness, improve skin firmness, reduce tactile roughness, improve skin texture, overall photo damage, overall hyperpigmentation, global improvement, reduce in appearance of dark spots and/or patches, improve appearance of skin brightness and youthful appearance, improve overall condition of skin, improve the appearance of photo aged skin, improve appearance of intrinsically and extrinsically aged skin, improve skin cellular turnover, improve skin barrier, improve skin's ability to retain moisture, reduce the appearance of brown and red blotchiness, redness, increase skin epidermal thickness, strengthen dermal epidermal junction, reduce the appearance of pore size and pores, improve smoothness, or a combination thereof.

Methods of treating a skin condition in a subject in need thereof provided herein comprise administering a composition comprising a heparin or a heparan sulfate isolated from a genetically modified cell line. In some cases, the genetically modified cell line comprises at least one of a mastocytoma (MST) cell line and a basophil neoplastic cell line. In some cases, the genetically modified cell line is deficient for one or more of chondroitin sulfate synthase 1 (CHSY1), chondroitin sulfate N-acetylgalactosaminyltransferase 2 (CSGALNACT2), chondroitin sulfate N-acetylgalactosaminyltransferase 1 (CSGALNACT1), Heparan sulfate 2-O-sulfotransferase (HS2ST), and heparan sulfate C5-epimerase (GLCE). In some cases, the genetically modified cell line is deficient for a heparan sulfate catabolic enzyme. In some cases, the heparan sulfate catabolic enzyme comprises one or more of heparanase (HPSE), beta-glucoronidase (GUSB), sulfamidase (SGSH), heparan alpha-glucosaminide N-acetyltransferase (HGNAT), alpha-N-acetyl glucosaminidase (GSNAT), uronate-2-O-sulfatases (IDS and GDS), alpha-L-iduronidase (IDUA), and heparan sulfate 6-O-endosulfatase (SULF1-2). In some cases, the genetically modified MST cell line overexpresses one or more of heparanase, a protease, a polymerase, a sulfotransferase, an endosulfatase, and a proteoglycan protein core. In some cases, the protease comprises one or more of matrix metalloproteinase (MMP), MMP2, MMP3, MMP7, MMP9, MT1-MMP, MT3-MMP, ADAM17, ADAMTS1, ADAMTS4, trypsin, and chymotrypsin. In some cases, the proteoglycan protein core comprises one or more of serglycin, a syndecan, a glypican, perlecan, and CD44 core protein (CD44E). In some cases, the serglycin comprises a human serglycin, a pig serglycin, a mouse serglycin. In some cases, the number of GAG attachment sites in the serglycin is modified. In some cases, the syndecan is selected from the group consisting of syndecan-1, syndecan-2, syndecan-3, syndecan-4, and syndecan ectodomains. In some cases, the glypican is selected from the group consisting of glypican 1, glypican 2, glypican 3, glypican 4, glypican 5, and glypican 6 and ectodomains.

### Heparin and Heparan Sulfate Compositions

Provided herein are compositions comprising heparin or heparan sulfate having reduced affinity for a platelet factor compared to unfractionated heparin. In some cases, the platelet factor is a platelet factor 4 (PF4). Such compositions herein often have a lower risk of complications often observed in subjects treated with unfractionated heparin. Heparin and heparan sulfate compositions herein are often isolated from genetically modified cell lines, such as mastocytoma cell lines or basophil neoplastic cell lines. In some cases, the genetically modified cell lines are selected from the group consisting of MST cells, P815 cells, MC/9 cells, SI/SI4 cells, 10P2 cells, 11P0-1 cells, and 10P12 cells. In some cases, the composition is purified from a mastocytoma (MST) cell line or a basophil neoplastic cell line genetically modified to be deficient for In some embodiments, the genetically modified cell line is RT4 cells, 682B cells, 751G cells, 1016T cells, KK-47, MGH-U1, MHG-U2, MGH-U3, or MGH-U4. Heparan sulfate 2-O-sulfotransferase (HS2ST). In some cases, the composition is purified from a MST cell line or a basophil neoplastic cell line genetically modified to overexpress Heparan sulfate-6-O-endosulfatase 1 and 2 (SULF1-2).

### High-Throughput Methods of Measuring Heparin and Heparan Sulfate

Also provided herein are high throughput methods of quantifying heparan sulfate in a group of samples. High throughput analysis of heparin and heparan sulfate samples is valuable in optimizing methods of production of heparin and heparan sulfate disclosed herein. In some embodiments, the methods comprise: (a) binding each sample to a well of a multi-well chromatography column; (b) digesting the samples bound to the column with an enzyme; (c) eluting the samples from the column with a solution comprising a salt; and (d) measuring the heparan sulfate in the sample using liquid chromatography. In some embodiments, the chromatography column is selected from at least one of an ion exchange column and a size exclusion column. In some embodiments, the enzyme is selected from at least one of a nuclease and a protease. In some embodiments, the salt is a volatile salt. In some embodiments, the liquid chromatography is an ultra performance liquid chromatography. In some embodiments, the method comprises liquid chromatography with fluorescently tagged heparan sulfate disaccharides.

Further provided herein are high throughput methods of quantifying heparan sulfate in a group of samples, the methods comprising: (a) contacting each sample to a well in a multi-well plate, wherein each well is coated with a guanidinylated antibiotic, thereby binding the heparan sulfate in the sample to the plate; (b) contacting the bound heparan sulfate to a heparan sulfate binding protein; (c) contacting the bound heparan sulfate binding protein to a detection reagent; and (d) measuring a signal from the detection reagent, wherein the signal from the detection reagent corresponds to the amount of heparan sulfate in the sample. In some embodiments, the guanidinylated antibiotic comprises guanidinylated neomycin. In some embodiments, the heparan sulfate binding protein is selected from at least one of FGF-2, PF4, ATIII, and VEGF. In some embodiments, the signal is selected from at least one of a fluorescent signal; a luminescent signal; and a colorimetric signal. In some embodiments, the signal is generated enzymatically.

Suitable heparin and heparan sulfate binding proteins for detection of heparin and heparan sulfate include but are not limited to 4F2 cell-surface antigen heavy chain (4F2hc); 5′-nucleotidase (5′-NT); Alpha-1-antitrypsin (Alpha-1 protease inhibitor); Alpha-1B-glycoprotein (Alpha-1-B glycoprotein); Alpha-2-macroglobulin (Alpha-2-M); Amyloid beta A4 protein (ABPP); Alpha-1-antichymotrypsin (ACT); Angio-associated migratory cell protein; Bile salt export pump (ATP-binding cassette sub-family B member 11); ATP-binding cassette sub-family G member 2 (CD antigen CD338); ATP-binding cassette sub-family G member 5 (Sterolin-1); Amiloride-sensitive amine oxidase [copper-containing] (DAO) (Diamine oxidase); Alpha-1B adrenergic receptor (Alpha-1B adrenoreceptor); Agouti-related protein; Aminoacyl tRNA synthase complex-interacting multifunctional protein 1 (Multisynthase complex auxiliary component p43); Aldose reductase (AR); Protein AMBP; Inter-alpha-trypsin inhibitor light chain (ITI-LC); Alpha-2-macroglobulin receptor-associated protein (Alpha-2-MRAP); Angiogenin (RNase 5); Angiotensinogen (Serpin A8); Antithrombin-III (ATIII); Annexin A1; Annexin A2; Annexin A3; Annexin A5; Annexin A6; Amyloid-like protein 1 (APLP-1); Amyloid-like protein 2 (APLP-2); Apolipoprotein A-V (Apo-AV); Apolipoprotein B-100 (Apo B-100); Apolipoprotein E (Apo-E); Beta-2-glycoprotein 1 (Beta(2)GPI); Aquaporin-1 (AQP-1); Arginase-1; Artemin; Agouti-signaling protein (ASP); Sodium/potassium-transporting ATPase subunit alpha-1 (Na(+)/K(+) ATPase alpha-1 subunit); Sodium/potassium-transporting ATPase subunit beta-1 (Sodium/potassium-dependent ATPase subunit beta-1); Sodium/potassium-transporting ATPase subunit beta-3 (Sodium/potassium-dependent ATPase subunit beta-3); Plasma membrane calcium-transporting ATPase 1 (PMCA1); Copper-transporting ATPase 2; ATP synthase subunit alpha, mitochondrial; Attractin (DPPT-L); A disintegrin and metalloproteinase with thrombospondin motifs 1 (ADAM-TS 1); A disintegrin and metalloproteinase with thrombospondin motifs 3 (ADAM-TS 3); A disintegrin and metalloproteinase with thrombospondin motifs 5 (ADAM-TS 5); A disintegrin and metalloproteinase with thrombospondin motifs 8 (ADAM-TS 8); A disintegrin and metalloproteinase with thrombospondin motifs 9 (ADAM-TS 9); Beta-2-microglobulin; Band 3 anion transport protein (Anion exchange protein 1); cDNA FLJ57339, highly similar to Complement C3; Beta-secretase 1; Bone morphogenetic protein 2 (BMP-2); Bone morphogenetic protein 3 (BMP-3); Bone morphogenetic protein 4 (BMP-4); Bone morphogenetic protein 6 (BMP-6); Bone morphogenetic protein 7 (BMP-7); Probetacellulin; Complement C1q subcomponent subunit A; Complement C1q subcomponent subunit B; Complement C1q subcomponent subunit C; C4b-binding protein alpha chain (C4bp); Voltage-dependent L-type calcium channel subunit alpha-1S; Cadherin-8; Azurocidin (Heparin-binding protein); Cathepsin B; Cathepsin G; Corticosteroid-binding globulin; Carboxypeptidase B2; Carboxypeptidase D; Coiled-coil domain-containing protein 134; Coiled-coil domain-containing protein 80; C-C motif chemokine 1; Eotaxin (C-C motif chemokine 11); C-C motif chemokine 13 (CK-beta-10); C-C motif chemokine 15 (Chemokine CC-2); C-C motif chemokine 17 (CC chemokine TARC); C-C motif chemokine 19; C-C motif chemokine 2 (HC11); C-C motif chemokine 21 (6Ckine); C-C motif chemokine 22 (CC chemokine STCP-1); C-C motif chemokine 23 (CK-beta-8); C-C motif chemokine 24 (CK-beta-6); C-C motif chemokine 25 (Chemokine TECK); C-C motif chemokine 27 (CC chemokine ILC); C-C motif chemokine 28 (Mucosae-associated epithelial chemokine); C-C motif chemokine 3; C-C motif chemokine 4; C-C motif chemokine 5 (EoCP); C-C motif chemokine 7 (Monocyte chemoattractant protein 3); C-C motif chemokine 8 (HC14); Fibronectin type-III domain-containing protein C4orf31; Antigen-presenting glycoprotein CD1d (R3G1) (CD antigen CD1d); Platelet glycoprotein 4; Leukocyte surface antigen CD47; Bile salt-activated lipase (BAL); Ceruloplasmin (Ferroxidase); Uncharacterized protein C6orf15 (Protein STG); Complement factor B; Complement factor D; Complement factor H (H factor 1); Complement factor I; Chordin; UPF0765 protein C10orf58; Clusterin; Chymase; Collagen alpha-1(I) chain (Alpha-1 type I collagen); Collagen alpha-2(I) chain (Alpha-2 type I collagen); Complement C2; Collagen alpha-1(II) chain; Complement C3; Collagen alpha-1(III) chain; Complement C4-A (Acidic complement C4); Collagen alpha-1(IV) chain; Collagen alpha-2(IV) chain; Complement C5; Collagen alpha-1(V) chain; Collagen alpha-3(V) chain; Complement component C6; Collagen alpha-3(VI) chain; Complement component C7; Complement component C8 alpha chain; Complement component C8 beta chain (Complement component 8 subunit beta); Complement component C8 gamma chain; Complement component C9; Collagen alpha-1(IX) chain; Collagen alpha-1(XI) chain; Collagen alpha-2(XI) chain; Collagen alpha-1(XII) chain; Collagen alpha-1(XIII) chain (COLXIIIA1); Collagen alpha-1(XIV) chain (Undulin); Collagen alpha-1(XVIII) chain; Collagen alpha-1(XIX) chain (Collagen alpha-1(Y) chain); Acetylcholinesterase collagenic tail peptide (AChE Q subunit); Cartilage oligomeric matrix protein (COMP) (Thrombospondin-5) (TSPS); Catechol O-methyltransferase; Collagen alpha-1(XXIII) chain; Collagen alpha-1(XXV) chain; Calcium release-activated calcium channel protein 1; Cysteine-rich secretory protein LCCL domain-containing 2; Granulocyte-macrophage colony-stimulating factor (GM-CSF); Connective tissue growth factor; Low affinity cationic amino acid transporter 2 (CAT-2); Gap junction beta-1 protein (Connexin-32); C-X-C motif chemokine 2; C-X-C motif chemokine 6 (Chemokine alpha 3); Platelet basic protein (PBP); C-X-C motif chemokine 10; C-X-C motif chemokine 11; C-X-C motif chemokine 13; C-X-C motif chemokine 16; Cytochrome c; Protein CYR61; Netrin receptor DCC; Estradiol 17-beta-dehydrogenase 11; Estradiol 17-beta-dehydrogenase 12; 17-beta-hydroxysteroid dehydrogenase 13; 3-keto-steroid reductase; Dipeptidyl peptidase 4; Endothelin-converting enzyme 1; Extracellular matrix protein 2; Ephrin-A1 (EPH-related receptor tyrosine kinase ligand 1); Ephrin-A3 (EFL-2); Ephrin-A5 (AL-1); Elastin (Tropoelastin); Neutrophil elastase; Alpha-enolase; Ectonucleotide pyrophosphatase/phosphodiesterase family member 1 (E-NPP 1); Ectonucleotide pyrophosphatase/phosphodiesterase family member 3 (E-NPP 3); Receptor tyrosine-protein kinase erbB-2; Coagulation factor X; Coagulation factor XI; Coagulation factor XII; Protein FAM55A; Coagulation factor IX; Fibulin-7 (FIBL-7); Fibrillin-1; Fibrillin-2; Fibrosin-1; IgG receptor FcRn large subunit p51 (FcRn); Fetuin-B; Heparin-binding growth factor 1 (HBGF-1); Fibroblast growth factor 10 (FGF-10); Fibroblast growth factor 12 (FGF-12); Fibroblast growth factor 14 (FGF-14); Fibroblast growth factor 16 (FGF-16); Fibroblast growth factor 17 (FGF-17); Fibroblast growth factor 18 (FGF-18); Heparin-binding growth factor 2 (HBGF-2) (bFGF); Fibroblast growth factor 2 (FGF-2); Fibroblast growth factor 20 (FGF-20); Fibroblast growth factor 22 (FGF-22); Fibroblast growth factor 3 (FGF-3); Fibroblast growth factor 4 (FGF-4); Fibroblast growth factor 5 (FGF-5); Fibroblast growth factor 6 (FGF-6); Keratinocyte growth factor (KGF); Fibroblast growth factor 8 (FGF-8); Glia-activating factor (GAF); Fibroblast growth factor-binding protein 1 (FGF-BP); Fibroblast growth factor-binding protein 3 (FGF-BP3); Basic fibroblast growth factor receptor 1 (FGFR-1); Fibroblast growth factor receptor 2 (FGFR-2); Fibroblast growth factor receptor 3 (FGFR-3); Fibroblast growth factor receptor 4 (FGFR-4); Fibrinogen alpha chain; Fibrinogen beta chain; Fibrinogen gamma chain; Fibronectin (FN); Follistatin (FS); Follistatin-related protein 1; Furin; Protein G6b; Glia-derived nexin (GDN); Glial cell line-derived neurotrophic factor (hGDNF); Gelsolin (AGEL); Growth hormone receptor (GH receptor); G-protein coupled receptor 182; Transmembrane glycoprotein NMB (Transmembrane glycoprotein HGFIN); Growth-regulated alpha protein (C-X-C motif chemokine 1); Solute carrier family 2, facilitated glucose transporter member 2 (GLUT-2); Proheparin-binding EGF-like growth factor; Hepatoma-derived growth factor (HDGF); Heparin cofactor 2 (Heparin cofactor II) (HC-II); Hereditary hemochromatosis protein (HLA-H); Hepatocyte growth factor (Hepatopoeitin-A); High mobility group protein B1 (HMG-1); Haptoglobin; Histidine-rich glycoprotein (HPRG); Islet amyloid polypeptide (Amylin); Insulin-like growth factor-binding protein 2 (IBP-2); Insulin-like growth factor-binding protein 3 (IBP-3); Insulin-like growth factor-binding protein 4 (IBP-4); Insulin-like growth factor-binding protein 5 (IBP-5); Insulin-like growth factor-binding protein 6 (IBP-6); Plasma protease C1 inhibitor (C1 Inh); Interferon gamma (IFN-gamma); Indian hedgehog protein (IHH); Interferon-inducible GTPase 5; Interleukin-10 (IL-10); Interleukin-12 subunit beta (IL-12B); Interleukin-2 (IL-2); Interleukin-3 (IL-3); Interleukin-4 (IL-4); Interleukin-5 (IL-5); Interleukin-6 (IL-6); Interleukin-7 (IL-7); Interleukin-8 (IL-8); Interphotoreceptor matrix proteoglycan 2; Inhibin beta A chain; Insulin receptor (IR); Plasma serine protease inhibitor; Integrin alpha-1; Integrin alpha-5; Integrin alpha-M; Integrin alpha-V; Integrin beta-1; Integrin beta-3; Inter-alpha-trypsin inhibitor heavy chain H3; Integral membrane protein 2B; Anosmin-1; Putative keratinocyte growth factor-like protein 1; Putative keratinocyte growth factor-like protein 2; Kininogen-1 (Alpha-2-thiol proteinase inhibitor); Laminin subunit alpha-1 (Laminin A chain); Laminin subunit alpha-2 (Laminin M chain); Laminin subunit alpha-3; Laminin subunit alpha-4; Laminin subunit alpha-5; Laminin subunit gamma-2; Leucyl-cystinyl aminopeptidase; Low-density lipoprotein receptor (LDL receptor); Galectin-9 (Gal-9); Leucine-rich repeat-containing G-protein coupled receptor 4 (G-protein coupled receptor 48); Leukemia inhibitory factor receptor (LIF receptor); Hepatic triacylglycerol lipase (HL); Endothelial lipase; Lipoprotein lipase (LPL); Platelet-activating factor acetylhydrolase IB subunit alpha (Lissencephaly-1 protein); Latrophilin-2; Latent-transforming growth factor beta-binding protein 1 (LTBP-1); L-selectin; P-selectin; Mannose-binding protein C (MBP-C); Multidrug resistance protein 1; Multidrug resistance protein 3; Hepatocyte growth factor receptor (HGF receptor); Macrophage migration inhibitory factor (MIF); Midkine (MK); Matrix metalloproteinase-14 (MMP-14); 72 kDa type IV collagenase; Matrilysin; Matrix metalloproteinase-9 (MMP-9); Monocarboxylate transporter 1 (MCT 1); Monocarboxylate transporter 8 (MCT 8); Multidrug resistance-associated protein 6 (ATP-binding cassette sub-family C member 6); Myosin regulatory light polypeptide 9; Neuron navigator 2; Neural cell adhesion molecule 1 (N-CAM-1); Netrin-1; Nicastrin; Noggin; Pro-neuregulin-1; Neuropilin-1; Neurturin; Sodium/bile acid cotransporter; Occludin; Zinc finger protein OZF; Calcium-dependent phospholipase A2; Phospholipase A2, membrane associated; Plasminogen activator inhibitor 1 (PAI); Plasminogen activator inhibitor 1 RNA-binding protein (PAI1 RNA-binding protein 1); Proton-coupled folate transporter (G21); Procollagen C-endopeptidase enhancer 2; Proprotein convertase subtilisin/kexin type 5; Proprotein convertase subtilisin/kexin type 6; Programmed cell death protein 5; Platelet-derived growth factor subunit A (PDGF subunit A); Platelet-derived growth factor subunit B (PDGF subunit B); Protein disulfide-isomerase (PDI); Protein disulfide-isomerase A6; Phosphatidylethanolamine-binding protein 1 (PEBP-1); Platelet endothelial cell adhesion molecule (PECAM-1); Pigment epithelium-derived factor (PEDF); Myeloperoxidase (MPO); Platelet factor 4 variant; Basement membrane-specific heparan sulfate proteoglycan core protein (Perlecan); Biglycan; Polymeric immunoglobulin receptor (PIgR); Putative phospholipase B-like 1; Platelet factor 4 (PF4); Placenta growth factor (P1GF); Plasminogen; Serum paraoxonase/arylesterase 1 (PON 1); Serum paraoxonase/arylesterase 2 (PON 2); Serum paraoxonase/lactonase 3 (EC 3.1.1.2) (EC 3.1.1.81) (EC 3.1.8.1); Periostin (PN); Peptidyl-prolyl cis-trans isomerase B (PPIase B); Peroxiredoxin-4; Prolargin; Bone marrow proteoglycan (BMPG); Major prion protein (PrP); Prolactin (PRL); Vitamin K-dependent protein C; Properdin (Complement factor P); Presenilin-1 (PS-1); Protein patched homolog 1 (PTC1); Pleiotrophin (PTN); Receptor-type tyrosine-protein phosphatase C; Stromal cell-derived factor 1 gamma; Liver-specific organic anion transporter 3TM13 (Organic anion transporter LST-3c); SLCO1A2 protein; Mannan-binding protein (Fragment); 60S ribosomal protein L22; 60S ribosomal protein L29 (Cell surface heparin-binding protein HIP); Roundabout homolog 1 (H-Robo-1); R-spondin-1 (hRspo1); R-spondin-2 (hRspo2); R-spondin-3 (hRspo3); R-spondin-4 (hRspo4); 40S ribosomal protein SA; Solute carrier family 12 member 9; Sodium-dependent phosphate transporter 2; Solute carrier family 22 member 1 (hOCT1); Solute carrier family 22 member 7 (hOAT2); Solute carrier family 22 member 18; Sodium-coupled neutral amino acid transporter 3 (N-system amino acid transporter 1); Sodium-coupled neutral amino acid transporter 4; Zinc transporter ZIP4; Electrogenic sodium bicarbonate cotransporter 1 (kNBC1); Serum amyloid A protein (SAA); Serum amyloid P-component (SAP); Sodium channel protein type 5 subunit alpha (HH1); Stromal cell-derived factor 1 (SDF-1); Semaphorin-5A; Semaphorin-5B; Secreted frizzled-related protein 1 (FRP-1); Sonic hedgehog protein (SHH); Beta-galactoside alpha-2,6-sialyltransferase 1; Slit homolog 1 protein (Slit-1); Slit homolog 2 protein (Slit-2); Antileukoproteinase (ALP); Synaptogyrin-1; Superoxide dismutase; Extracellular superoxide dismutase; Sortilin; Sclerostin; Stabilin-2; Metalloreductase STEAP4; Stromal interaction molecule 1; Alpha-synuclein; Microtubule-associated protein tau; Teneurin-1 (Ten-1); Tenascin (TN); Tenascin-X (TN-X); Tissue factor pathway inhibitor (TFPI); Transferrin receptor protein 1 (TR); Transferrin receptor protein 2 (TfR2); Transforming growth factor beta receptor type 3; Transforming growth factor beta-1 (TGF-beta-1); Transforming growth factor beta-2 (TGF-beta-2); Protein-glutamine gamma-glutamyltransferase 2 (TGase-2); Thioredoxin (Trx); Prothrombin; Thyroglobulin (Tg); Metalloproteinase inhibitor 3; T-cell immunomodulatory protein (Protein TIP); Tumor necrosis factor ligand superfamily member 13; Tumor necrosis factor (TNF-alpha); Tissue-type plasminogen activator (t-PA); Tumor necrosis factor receptor superfamily member 11B; Serotransferrin (Transferrin); Lactotransferrin (Lactoferrin); Trypsin-1; Tryptase alpha/beta-1 (Tryptase-1); Tryptase beta-2 (Tryptase-2); Tumor necrosis factor-inducible gene 6 protein; Thrombospondin-1; Thrombospondin-2; Thrombospondin-3; Thrombospondin-4; Transthyretin (ATTR); Urokinase-type plasminogen activator (uPA); Vascular endothelial growth factor (VEGF); Vascular endothelial growth factor A (VEGF-A); Vascular endothelial growth factor B (VEGF-B); Vascular endothelial growth factor receptor 1 (VEGFR-1); Vascular endothelial growth factor receptor 2 (VEGFR-2); Vitamin D-binding protein (DBP); Vitronectin; von Willebrand factor (vWF); Proto-oncogene Wnt-1; Fractalkine (C-X3-C motif chemokine 1); Lymphotactin; Xanthine dehydrogenase/oxidase; Zinc transporter 1 (ZnT-1); and Protein Z-dependent protease inhibitor (PZI).

### Treatment and Prevention of Viral Infection

In some embodiments, there is provided a method of treating or preventing a viral infection in a subject in need thereof comprising administering to the subject an effective amount of one or more heparin or heparan sulfate compositions produced by methods described herein. In some embodiments, the heparin or heparan sulfate inhibits viral attachment to a cell. In some embodiments, the heparin or heaparan sulfate lacks anti-coagulant or anti-clotting activity. In some embodiments, the viral infection comprises a Adenoviridae such as, an Adenovirus; a Herpesviridae such as a Herpes simplex, type 1, a Herpes simplex, type 2, a Varicella-zoster virus, an Epstein-barr virus, a Human cytomegalovirus, a Human herpesvirus, type 8; a Papillomaviridae such as a Human papillomavirus; a Polyomaviridae such as a BK virus or a JC virus; a Poxviridae such as a Smallpox; a Hepadnaviridae such as a Hepatitis B virus; a Parvoviridae such as a Human bocavirus or a Parvovirus; a Astroviridae such as a Human astrovirus; a Caliciviridae such as a Norwalk virus; a Picornaviridae such as a coxsackievirus, a hepatitis A virus, a poliovirus, a rhinovirus; a Coronaviridae such as a Severe acute respiratory syndrome virus or a COVID-19 virus; a Flaviviridae such as a Hepatitis C virus, a yellow fever virus, a dengue virus, a West Nile virus; a Togaviridae such as a Rubella virus; a Hepeviridae such as a Hepatitis E virus; a Retroviridae such as a Human immunodeficiency virus (HIV); a Orthomyxoviridae such as an Influenza virus; a Arenaviridae such as a Guanarito virus, a Junin virus, a Lassa virus, a Machupo virus, a Sabia virus; a Bunyaviridae such as a Crimean-Congo hemorrhagic fever virus ; a Filoviridae such as a Ebola virus, a Marburg virus; a Paramyxoviridae such as a Measles virus, a Mumps virus, a Parainfluenza virus, a Respiratory syncytial virus, a Human metapneumovirus, a Hendra virus, a Nipah virus; a Rhabdoviridae such as a Rabies virus; a Hepatitis D virus; or a Reoviridae such as a Rotavirus, a Orbivirus, a Coltivirus, a Banna virus infection. In some embodiments, treatment or prevention of the viral infection reduces one or more symptoms such as fever, diarrhea, fatigue, shortness of breath, or pain.

### Definitions

The term “glycosaminoglycan” or “GAG” as used herein refers to long unbranched polysaccharides consisting of a repeating disaccharide unit. The repeating unit (except for keratan) consists of an amino sugar (N-acetylglucosamine or N-acetylgalactosamine) along with a uronic sugar (glucuronic acid or iduronic acid) or galactose.

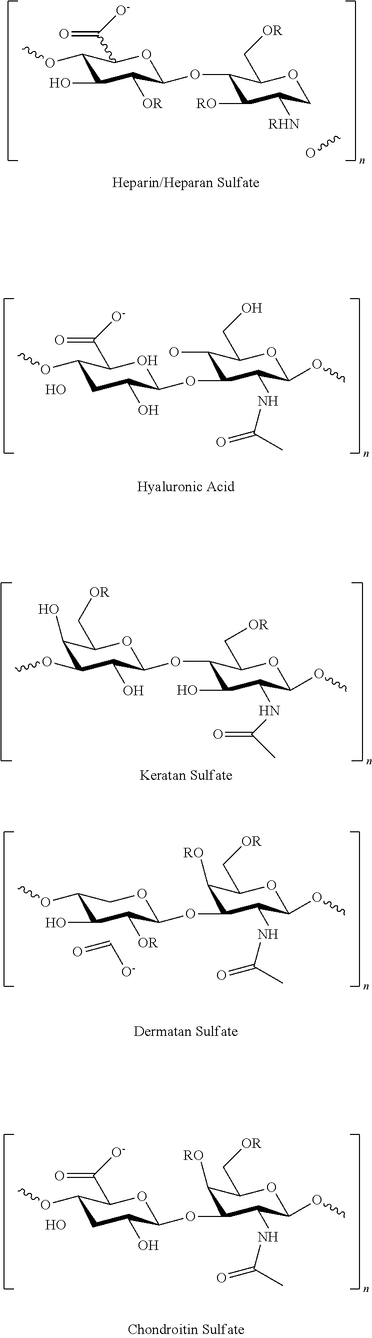

The term “proteoglycan” as used herein refers to proteins that are heavily glycosylated. The basic proteoglycan unit comprises a core protein with one or more covalently attached glycosaminoglycan or GAG chains.

The term “core protein” as used herein refers to a protein component of a proteoglycan.

The term “heparin” as used herein refers to a glycosaminoglycan made of repeating disaccharide units comprising one or more of β-D-glucuronic acid (GlcA), 2-deoxy-2-acetamido-α-D-glucopyranosyl (GlcNAc), α-L-iduronic acid (IdoA), 2-O-sulfo-α-L-iduronic acid (IdoA2S), 2-deoxy-2-sulfamido-α-D-glucopyranosyl (GlcNS), 2-deoxy-2-sulfamido-α-D-glucopyranosyl-6-O-sulfate (GlcNS6S) or 2-deoxy-2-sulfamido-α-D-glucopyranosyl-3,6-O-disulfate (GlcNS3S6S) or 2-deoxy-2-sulfamido-α-D-glucopyranosyl-3-O-sulfate (GlcNS3S).

The term “heparan sulfate” as used herein refers to a linear polysaccharide with the structure. Heparan sulfate is made of repeating disaccharide units. The repeating disaccharide units can comprise one or more of β-D-glucuronic acid (GlcA), 2-deoxy-2-acetamido-α-D-glucopyranosyl (GlcNAc), α-L-iduronic acid (IdoA), 2-O-sulfo-α-L-iduronic acid (IdoA2S), 2-deoxy-2-sulfamido-α-D-glucopyranosyl (GlcNS), 2-deoxy-2-sulfamido-α-D-glucopyranosyl-6-O-sulfate (GlcNS6S) or 2-deoxy-2-sulfamido-α-D-glucopyranosyl-3,6-O-disulfate (GlcNS3S6S) or 2-deoxy-2-sulfamido-α-D-glucopyranosyl-3-O-sulfate (GlcNS3S).

The term “chondroitin sulfate” as used herein refers to a linear polysaccharide with the structure. Chondroitin sulfate is made of repeating dissacharide units. The repeating disaccharide units can comprise one or more of N-acetylgalactosamine (GalNAc), N-acetylgalactosamine-4-sulfate (GalNAc4S), N-acetylgalactosamine-6-sulfate (GalNAc6S), N-acetylgalactosamine-4,6-disulfate (GalNAc4S6S) and β-D-glucuronic acid (GlcA), D-glucuronic acid-2-sulfate (GlcA2S), D-glucuronic acid-3-sulfate (GlcA3S), L-iduronic acid (IdoA), L-iduronic acid-2-sulfate (IdoA2S).

The terms “sulfation pattern”, “defined pattern of sulfation”, and “defined modification pattern” as used herein refer to enzymatic modifications made to the glycosaminoglycan including but not limited to include sulfation, deacetylation, and epimerization. This also includes heparin and heparan sulfate compositions having a defined disaccharide composition.

The term “genetically modified cell line” as used herein refers to a cell line with specific modifications made to the genome of the cell line. In some embodiments, the cell line is mammalian. In some embodiments, the cell line is human or murine. In some embodiments, the modifications comprise genetic knockouts, whereby the cell line becomes genetically deficient for one or more genes. In some embodiments, the modifications comprise making transgenic cell lines, whereby the cell line obtains genetic material not present in the wildtype cell line or genetic material under the control of active promoter.

The term “genetically deficient” as used herein refers to a genome that is modified to be missing one or more genes of interest. In some embodiments, the modification is made using a cre/lox system, CRISPR, siRNA, shRNA, antisense oligonucleotide, miRNA, or other genetic modification or mutagenesis method known in the art.

The term “transgenic” as used herein refers to a genome that is modified to include additional genetic material encoding one or more genes of interest. In some embodiments, the modification is made using transfection, infection with a virus, cre/lox knock-in, CRISPR/cas mediated knock-in, or other method of introducing genetic material to a cell that is known in the art.

The terms “subject”, “individual”, “recipient”, “host”, and “patient”, are used interchangeably herein and refer to any mammalian subject for whom diagnosis, treatment or therapy is desired, particularly humans. “Mammal” for purposes of treatment refers to any animal classified as a mammal, including humans, domestic and farm animals, and laboratory, zoo, spots, or pet animals, such as dogs, horses, cats, cows, sheep, goats, pigs, mice rats, rabbits, guinea pigs, monkeys, etc. In some embodiments, the mammal is human.

As used herein, the terms “treatment”, “treating” and the like, refer to administering an agent or carrying out a procedure, for the purposes of obtaining an effect. The effect may be prophylactic in terms of completely or partially preventing a disease or symptom thereof and/or may be therapeutic in terms of effecting a partial or complete cure for a disease and/or symptoms of the disease. “Treatment”, as used herein, may include treatment of a disease in a mammal, particularly in a human and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease. Treating may refer to any indicia of success in the treatment or amelioration or prevention of a disease, including any objective or subjective parameter such as abatement; remission; diminishing of symptoms or making the disease more tolerable to the patient; slowing in the rate of degeneration or decline; or making the final point of degeneration with less debilitation. The treatment or amelioration of symptoms can be based on objective or subjective parameters; including the results of an examination by a physician. Accordingly, the term “treating” includes the administration of the compounds or agents disclosed hereinto prevent or delay, to alleviate, or to arrest or inhibit development of the symptoms of conditions associated with the disease. The term “therapeutic effect refers to the reduction, elimination, or prevention of the disease, symptoms of the disease, or side effects of the disease in the subject.

“In combination with”, “combination therapy” and “combination products” refer, in certain embodiments, to concurrent administration to a patient of a first therapeutic and the compounds used herein. When administered in combination, each component can be administered at the same time or sequentially in any order at different points in time. Thus, each component can be administered separately but sufficiently closely in time as to provide the desired therapeutic effect.

“Dosage unit” refers to physically discrete units suited as unitary dosages for the particular individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or in the case of an aerosol composition, gaseous.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

A “therapeutically effective amount” means that the amount that, when administered to a subject for treating a disease, is sufficient to effect treatment for that disease.

The term “substantially free” as used herein means most or all of one or more of a contaminant, such as the materials with which it typically associates with in nature, is absent from the composition. Thus a heparin or heparan sulfate composition with defined modification patterns described herein that is “substantially free” from one or more contaminating glycosaminoglycans that do not have the desired defined modification pattern and/or biological and/or therapeutic effect has no or little of the contaminant. For example, a heparan sulfate composition is “substantially free” from a contaminant such as other glycosaminoglycans such as: chondroitin sulfate, keratan sulfate and/or hyaluronic acid; nucleic acids; and/or proteins, found with the heparan sulfate composition in nature, has very little or none of the contaminant, for example less than 5%, less than 4%, less than 3%, less than 2%, less than 1%, or less than 0.5% of the composition is made up by the contaminant. In some embodiments, the composition is 60%, 70%, 80%, 85%, 90%, 95%, 96%, 97%, 98%, 99%, or 100% free from one or more of a contaminating glycosaminoglycan, nucleic acids, and or proteins. In some embodiments, the composition is at least 95% free from contaminating glycosaminoglycans, nucleic acids, and or proteins. In some embodiments, the composition is at least 99% free from contaminating glycosaminoglycans, nucleic acids, and or proteins.

The term “substantially pure” as used herein means that the composition is free of most or all of the materials with which it typically associates with in nature. Thus a “substantially pure” glycosaminoglycan and/or heparan sulfate composition with defined modification patterns described herein does not include other contaminating glycosaminoglycan and/or heparan sulfate compositions that do not have the desired defined modification pattern and/or biological and/or therapeutic effect. For example, a “substantially pure” heparan sulfate composition is free from most other glycosaminoglycans such as: chondroitin sulfate, keratan sulfate and/or hyaluronic acid; nucleic acids; and/or proteins, found with the heparan sulfate composition in nature. In some embodiments, the composition is 60%, 70%, 80%, 85%, 90%, 95%, 96%, 97%, 98%, 99%, or 100% free from contaminating glycosaminoglycans, chondroitin sulfate, dermatan sulfate, keratan sulfate, nucleic acids, and or proteins. In some embodiments, the composition is 95% free from contaminating glycosaminoglycans, chondroitin sulfate, dermatan sulfate, keratan sulfate, nucleic acids, and or proteins. In some embodiments, the composition is 99% free from contaminating glycosaminoglycans, chondroitin sulfate, dermatan sulfate, keratan sulfate, nucleic acids, and or proteins. In some embodiments, the composition is greater than 99% free from contaminating glycosaminoglycans, chondroitin sulfate, dermatan sulfate, keratan sulfate, nucleic acids, and or proteins.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Cellular Production of Heparin in MST Cells

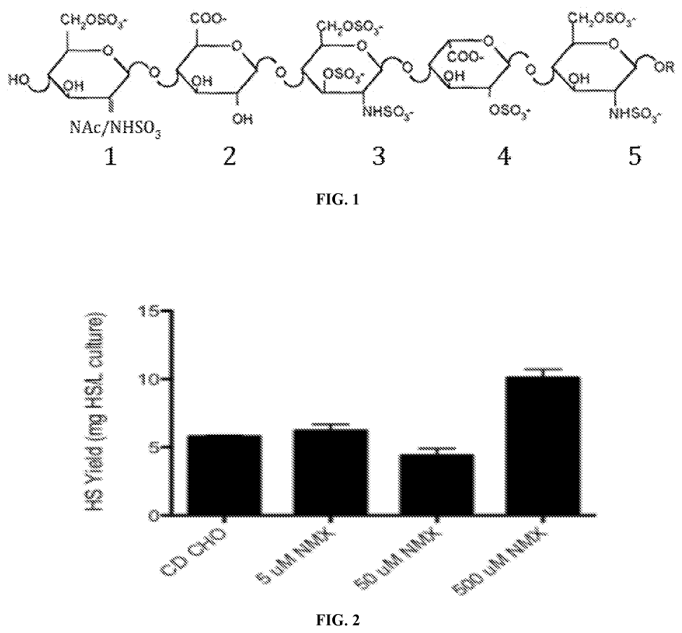

Producing recombinant heparin entails a higher level of complexity than producing a recombinant protein. Unlike recombinant proteins that are expressed by a single gene, heparin is synthesized in a complex metabolic pathway involving over 20 enzymes. Most cells produce heparan sulfate (HS), a similar polysaccharide, produced by the same biosynthetic pathway, but with a lower sulfate content and 100-1000-fold less anticoagulant potency. Heparin's anticoagulant properties critically depend on sulfate group additions to specific sugar residues along the polysaccharide chains, enabling heparin to bind and activate antithrombin. These sulfate modifications in turn, depend on the activities of specific sulfotransferase enzymes in the biosynthetic pathway. Unlike cell surface/extracellular matrix heparan sulfate, heparin polysaccharides are uniquely produced in mast cells where they are stored in cytoplasmic granules to be released along with inflammatory mediators upon degranulation. As primary mast cells are difficult to propagate and maintain, cellular production of heparin was conducted in MST cells, a stable clonal line derived from the Furth murine mastocytoma. MST cells are exceptional in producing highly sulfated polysaccharide chains in cytoplasmic granules similar to heparin, however they lack critical 3,6-O-sulfo-glucosamine residues required for anticoagulant activity. Presumably this is due to the minute levels of heparan sulfate 3-O-sulfortransferase 1 (Hs3st1) expressed in MST cells (undetectable by Western blotting) (Gasimli, L., Glass, C. A., Datta, P., Yang, B., Li, G., Gemmill, T. R., Baik, J. Y., Sharfstein, S. T., Esko, J. D., and Linhardt, R. J. (2014) Bioengineering murine mastocytoma cells to produce anticoagulant heparin. Glycobiology 24, 272-280). To compensate for this lack of activity, the MST cells were stably transfected using a retroviral vector containing the Hs3st1 sequence. Upon transfection, the new cell line (MST-10H) expressed key heparin biosynthetic enzymes, N-deacetylase-N-sulfotransferase 2 (NDST2), heparan sulfate 6-O-sulfotransferase 1 (HS6ST1), heparan sulfate 2-O-sulfotransferase (HS2ST) and HS3ST1 that are required for the formation of the antithrombin 3 (AT3) binding pentasaccharide motif (FIG. 1). Polysaccharide chains associated with these cells closely resemble heparin with appropriately positioned 3-O-sulfo group-containing glucosamine residues and significant anticoagulant potency. Similarities in composition and structure between commercial anticoagulant heparin and heparin produced by the MST-10H cells suggests that the MST-10H cells may be an excellent source of cell produced heparin.

### Example 2: GAG Yield and Viable Cell Density from MST Cells

Method: MST cells were grown in 30 mL or 1 L shaker flasks in CD CHO (with and without supplementation with Efficient Feed B) or in DMEM/F12+15% FBS. The viable cell density was determined on various days. The culture was harvested on day 10.

Glycosaminoglycans associated with the cell pellet were released by incubation with Pronase and Triton X-100. The glycosaminoglycans from both the cell pellet and the conditioned medium were purified on a DEAE-Sephacel column followed by digestion of contaminants with DNase and Pronase. The digested product was purified on DEAE again and desalted on PD10 columns. The yield of GAG purified from the medium was determined using the carbazole assay. In some cases, the glycosaminoglycan was exhaustively digested with heparin lyases or chondroitinase ABC and the amount of product formed was measured by absorbance at 232 nm.

Results: In 1 L flasks, the MST cells density reached a maximum on day 7. MST cells attained a higher cell density in DEME/F12 medium than in CD CHO medium. Maximum viable cell density was 2.58×10⁶ cells/mL in the DMEM/F12 and 0.94×10⁶ cells/mL in CD CHO medium (FIG. 2).

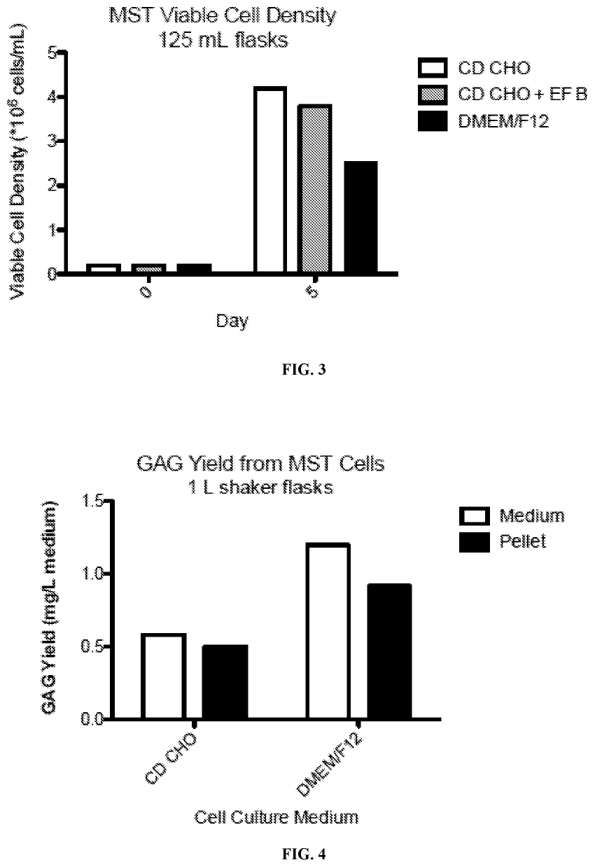

In 125 mL flasks, the CD CHO medium produced the maximum viable cell density on day 5, which was 4.2×10⁶ cells/mL. CD CHO plus supplementation with Efficient Feed B gave a viable cell density of 3.8×10⁶ cells/mL and DMEM/F12 gave a viable cell density of 2.5×10⁶ cells/mL (FIG. 3).

In 1 L flasks, the GAG in MST cells grown in CD CHO medium was distributed roughly equally between the medium and the cell pellet. For growth in DMEM/F12+15% serum, the medium yielded 1.2 mg of GAG per L in the medium and 0.9 mg of GAG per L in the cell pellet (FIG. 4).

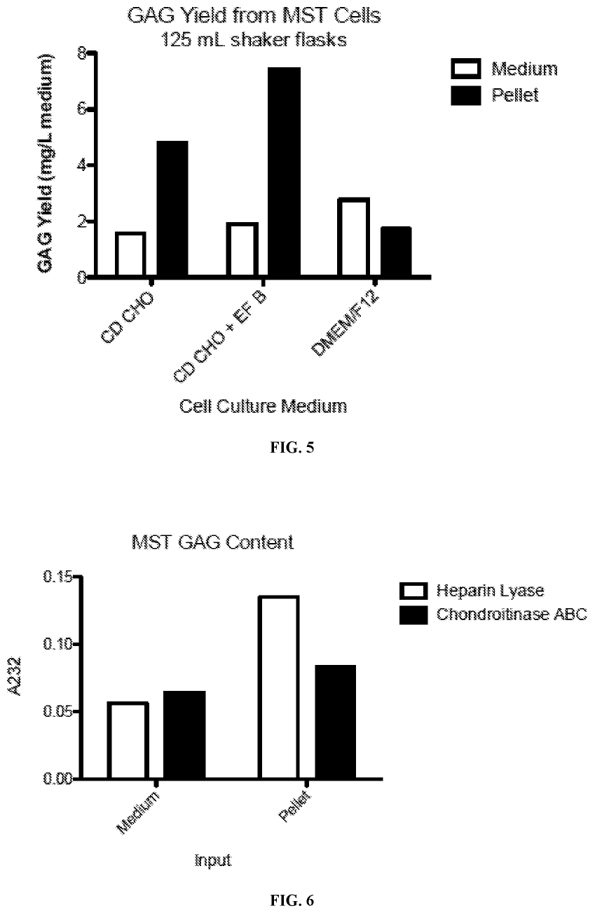

For cells grown in the 125 mL flasks, the yields of GAG per cell culture volume were higher than in the 1 L flasks. A larger proportion of the GAG was found in the cell pellet in the CD CHO medium whereas a lesser portion was found in the cell pellet in DMEM/F12 cells (FIG. 5).

For MST cells grown in CD CHO medium plus Efficient Feed B supplement, the HS/CS content was roughly equal in the GAG purified from the medium. For the GAG purified from the pellet, roughly 60 percent was heparan sulfate. Overall, two-thirds of the GAG was found in the cell pellet (FIG. 6)

TABLE 3
MST GAG Content - % of total

| | Cell Pellet | Medium | Total |
|---|---|---|---|
| Heparan Sulfate | 39.9 | 16.6 | 56.5 |
| Chondroitin Sulfate | 24.6 | 18.9 | 43.5 |
| Total GAG | 64.5 | 35.5 | 100 |

### Example 3: MST HS Disaccharide Content

Method: Purified MST HS was digested exhaustively with heparin lyases. Then, the digest was dried and aniline was chemically conjugated to the disaccharides. The aniline-disaccharide profile was determined by LCMS by comparison to isotopically labeled disaccharide standards. Pharmaceutical heparin was analyzed in parallel for comparison.

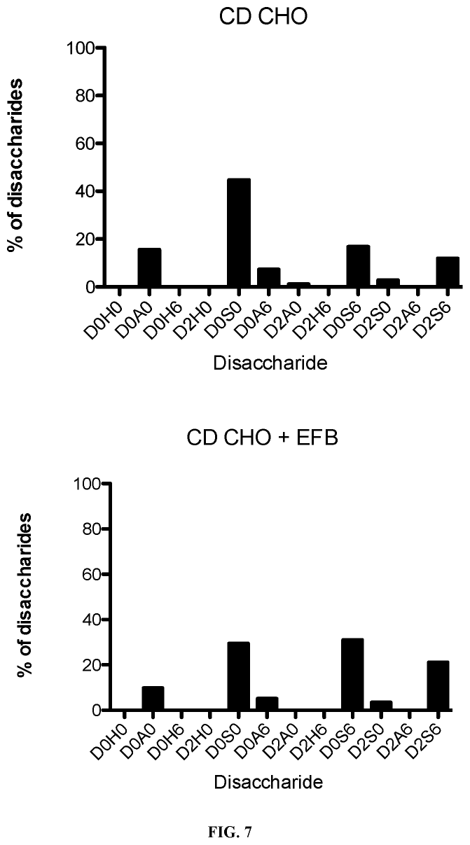

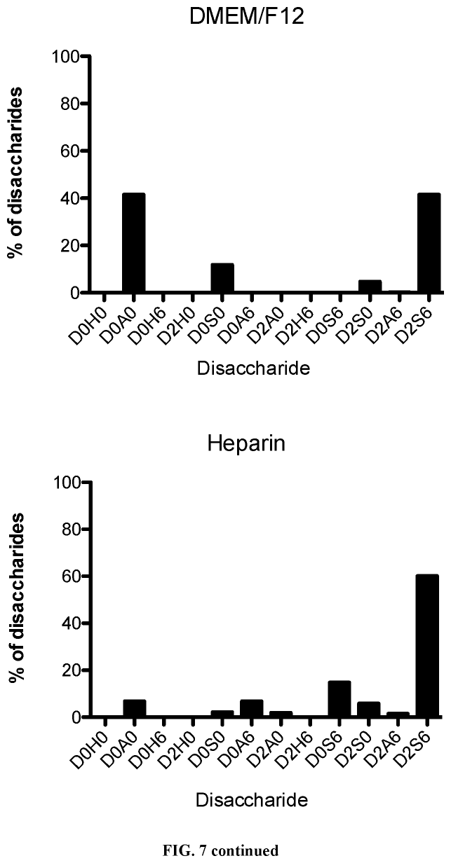

Results: The disaccharide composition differs based on the type of medium used to grow the cells. Of particular importance is the trisulfated disaccharide, D2S6, which is abundant in pharmaceutical heparin at more than 60 percent of the total disaccharides. DMEM/F12 medium plus 15% FBS gives heparan sulfate with a D2S6 content most similar to pharmaceutical heparin (FIG. 7).

### Example 4: MST-10H Anticoagulant Activity

Method: Purified MST and MST -10H heparan sulfate was used in a Factor Xa inactivation assay that depends on antithrombin binding to heparan sulfate to inactivate Factor Xa. Pharmaceutical heparin was used in parallel as standards. The extent of Factor Xa inactivation was determined by loss of signal from a fluorescent substrate.

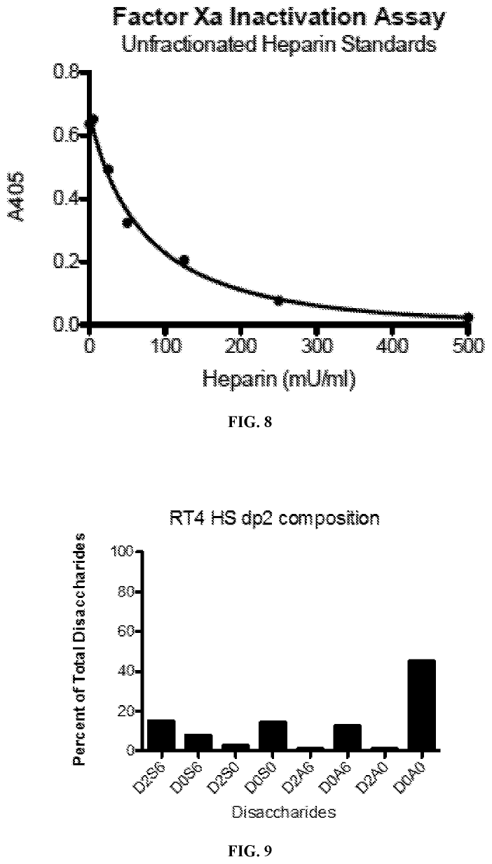

Results: The Factor Xa signal given by known concentrations of MST and MST-10H heparan sulfate was compared to the standard curve to calculate the specific activity of the heparan sulfate. MST heparan sulfate had a specific anti-Factor Xa activity of 0.61 U/mg heparan sulfate while MST-10H heparan sulfate had 35.1 U/mg of heparan sulfate. Pharmaceutical heparin by way of comparison has ˜180 U of anti-Factor Xa activity per mg of heparin (FIG. 8).

TABLE 4
Specific anti-Factor Xa activity (U/mg) of MST and MST-10H HS

| | MST | MST-10H |
|---|---|---|
| Anti-FXa activity (U/mg HS) | 0.61 | 35.1 |

### Example 5: Heparin from Engineered MST Cells

Retroviral transduction is used to overexpress additional heparan sulfate biosynthetic enzymes (Hs6st1 and Hs6st2) in MST cells. A retroviral transduction system with multiple expression vectors encoding different antibiotic resistance cassettes (such as, pQCXIN—G418, pQCXIH—hygromycin, pQCXIP—puromycin), which enables antibiotic selection for overexpression of three transgenes simultaneously. A packaging system, such as the Retro-X Universal Packaging System with the GP2-293 packaging cell line enables production of viruses with envelope proteins for pantropic or ecotropic infection of cell lines. Coding sequences, currently in other expression systems, are spliced into the multiple cloning sites of retroviral vectors pQCXIN, pQCXIH or pQCXIP (Clonetech). These vectors are cotransfected into the GP2-293 packaging cell line along with the pVSV-G (pantropic) packaging vector using the Xfect Transfection reagent (Clontech). Virus is harvested 48 hours after transfection and titered using the Retro-X qRT-PCR Titration Kit (Clontech).

MST-10H cells are cultured in DMEM/F12+15% FBS and transduced in suspension using a spinoculation protocol. Briefly, MST-10H cells are diluted to 1×10⁵ cells/ml, mixed with retrovirus at various multiplicities of infection (e.g. 0, 0.1, 0.5, 1.0, 5.0, 10.0), centrifuged at 800×g for 30 minutes at room temperature, resuspended in growth medium and transferred to 6-well dishes. After three days, the medium is replaced with antibiotic selection medium and the cells maintained until cell viability returns to >95%. We have already determined MST dose response curves for G418, hygromycin and puromycin. Following transfection cells are returned to serum free defined medium. Prior to HS isolation, a linear salt gradient is run to identify elution conditions for best selecting the high affinity heparin chains.

### Example 6: Chondroitin Sulfate Knockout

CRISPR/Cas9 is used to knock out the chondroitin sulfate (CS) branch of the GAG synthesis pathway in the MST-10H cells. This method was used to knock out the CS pathway in CHO cells and similar procedures are used here. In CHO cells, targeting either of two CS biosynthetic genes, chondroitin sulfate synthase I (CHSY1) or chondroitin sulfate N-acetylgalactosaminyltransferase 2 (CSGALNACT2), resulted in complete elimination of CS. An RNAseq analysis has identified expression of the corresponding mouse genes in the MST cells. CHSY1 is initially targeted as CHO cells with this gene inactivated had the highest HS production levels. The gene is inactivated by transfection with vectors expressing Cas9 and specifically targeted sgRNA (single guide RNA). Multiple targeting sequences are used for each gene. SURVEYOR analyses are used to reveal doublet bands indicative of indel (insertion/deletion) mutations. CS in fixed, permeabilized (BD Cytofix/Cytoperm Kit: #554714) cell populations are digested with chondroitinase ABC and subjected to flow cytometry with the anti-CS antibody 2B6 (detects the 4-O-sulfated CS stub remaining after chondroitinase digest) to estimate the inactivation frequency. Transfected cells are plated in limiting dilutions (1 cell/well in 96-well plates) to obtain single cell colonies. Colonies are analyzed by flow cytometry as described above and for the absence of CS verified by GAG quantification after digestion with chondroitinase or heparin lyases.

### Example 7: Increased Degranulation

Degranulation agents are tested for the ability to stimulate heparin release into the growth medium while maintaining cell proliferative capacity. The various agents are added to the cells growing in 6-well plates with and without agitation. Compound 48/80 and A23187 are added to the cell growth medium. TPA and Substance P are added to the cells in calcium & magnesium free-Hepes buffered salt solution because they are ineffective in calcium containing solutions (Jozaki, K., Kuriu, A., Waki, N., Adachi, S., Yamatodani, A., Tarui, S., and Kitamura, Y. (1990) Proliferative potential of murine peritoneal mast cells after degranulation induced by compound 48/80, substance P, tetradecanoylphorbol acetate, or calcium ionophore A23187. J Immunol 145, 4252-4256). After the prescribed incubation period samples of the cells and media are taken for analysis and the remaining cells are returned for continued growth. Heparin quantities in the medium are determined using heparin ELISA. Cell samples are stained with neutral red for counting the number of intact granules. May-Grunwald-Giemsa staining will be used for further morphological analysis of the degranulated cells if necessary. Cell viability and proliferation is monitored over the next 3-5 days by staining with trypan blue. Multiple agents could be tested in combination. Initial conditions are outlined in Table 5.

TABLE 5
Degranulation Agents

| | | |
|---|---|---|
| | Agent | |
| Agent ID | Concentration | Incubation Time |

| | | | |
|---|---|---|---|
| Cmpd 48/80 | 1 | μg/ml | 30 minutes |
| Cmpd 48/80 | 5 | μg/ml | 30 minutes |
| Cmpd 48/80 | 10 | μg/ml | 30 minutes |
| A23187 | 0.1 | μg/ml | 15 minutes |
| A23187 | 0.5 | μg/ml | 15 minutes |
| A23187 | 1.0 | μg/ml | 15 minutes |
| TPA | 1 | ng/ml | 60 minutes |
| TPA | 10 | ng/ml | 60 minutes |
| TPA | 30 | ng/ml | 60 minutes |
| Substance P | 50 | μM | 45 minutes |
| Substance P | 100 | μM | 45 minutes |
| Substance P | 200 | μM | 45 minutes |

| | | |
|---|---|---|
| No Agent | N/A | N/A |

### Example 8: Modified Heparin for Reduced Risk of Heparin Induced Thrombocytopenia

CRISPR/Cas9 is used to knock out Hs2st in the MST-10H cells. Since FGF2 binding to heparin is highly dependent on HS2ST, screening knockout colonies is accomplished with FGF2 binding to permeabilized cells using flow cytometry. Heparin from these cells is evaluated by disaccharide compositional analysis, PF4 and AT3 binding, factor Xa inhibition and assays for HIT. PF4 binding is also reduced by overexpressing SULF1-2 in the MST-10H cells using retroviral transduction. As described above, cell lines are also produced that lack HS2ST and overexpress SULF1-2. Heparin from these cells is evaluated by disaccharide compositional analysis, PF4 and AT3 binding, factor Xa inhibition. Heparin samples are also evaluated for HIT by platelet aggregation assays as well as various anticoagulation assays.

In addition to characterization described above, serotonin-release assays are used to detect anti-PF4/heparin antibodies in patient serum to diagnose HIT.

Engineered cell lines that are advanced to production are cloned and screened for high production capacity and stability. Screening clonal variation is an important, routine selection step used in industry for optimizing production. As recommended, high throughput ELISA protocols are used with appropriate ligands (e.g. FGF2) to assess production in at least 10,000 clones. Production in cell lines with the best characteristics is scaled up. Working cell lines are banked and used to produce a master cell line. Material from the master cell line is used in further chemistry manufacturing controls (CMC), efficacy and safety studies to prepare for a pre-IND meeting with the FDA and advancement to IND enabling studies.

### Example 9: Heparan Sulfate from RT4 Cells

Methods

RT4 cells (ATCC HTB-2), derived from a human urinary bladder papilloma, were grown in McCoy's Medium 5A+10% FBS. The adherent cell layer was washed with PBS and then lysed/digested with DEAE wash buffer (50 mM NaOAc, pH 6, 250 mM NaCl)+0.1% Triton-X100+0.5 mg/mL Pronase. The clarified digest was fractionated on DEAE-Sephacel, and then DNA and chondroitin sulfate were digested by addition of DNase and chondroitinase ABC. The mass of the HS was determined by the carbazole assay. The anti-FXa activity of the HS was determined using the FXa activity assay. The purified HS was digested exhaustively using a mixture of heparin lyases I, II and III. The resulting disaccharides were tagged with 2-aminoacridone and disaccharide composition was determined by UPLC with fluorescence detection.

Results

Notably, the anti-FXa activity of HS from these cells is higher than any other cellular source of HS that we have encountered (132 U/mg). By way of comparison, CHO cells with Hs3st1 overexpressed have 1 U/mg of activity and MST cells with Hs3st1 overexpressed have 80 U/mg of activity. Furthermore, the sulfate content of RT4 HS (97 SO₄ per 100 disaccharides) is much lower than pharmaceutical heparin (228 SO₄ per 100 disaccharides) or MST+Hs3st1 HS (229 SO₄ per 100 disaccharides). The lower sulfate content may result in less platelet factor 4 binding and reduced incidence of heparin induced thrombocytopenia with respect to pharmaceutical heparin. The disaccharide composition of RT4 cells is shown in FIG. 9.

### Example 10: Production of Heparan Sulfate by Engineered MST Cell Lines

This study focused on the development of a genetically engineered cell line with the heparan sulfate (HS) biosynthetic pathway tailored to the production of anticoagulant recombinant HS (rHS). This product would serve as a replacement for pharmaceutical heparin derived from porcine intestine. It was also intended to reduce the risk of heparin-induced thrombocytopenia (HIT) by modifying HS biosynthesis to reduce binding of platelet factor 4 (PF4). Finally, the efforts in bioprocess development were directed at increasing production levels through genetic engineering, medium formulation and optimization of growth conditions. This example outlines the results in each of these areas.

A mastocytoma cell line (MST) was determined to be much better suited to production of anticoagulant rHS. MST cells naturally produce HS with higher sulfate content than CHO, which approximates the sulfate content of pharmaceutical heparin. HS from MST cells lacks 3-O-sulfate, a modification that is required for anticoagulant activity. Otherwise, this cell line was a promising starting point to make a heparin substitute because of the natural similarity of its HS to pharmaceutical heparin. Since MST cells are not commonly used for production, several technical issues had to be addressed to adopt the cells for production. MST cells produce chondroitin sulfate (CS), a contaminant that co-purifies with HS. Also, instead of secreting HS (and CS) into the medium, MST cells store these glycosaminoglycans in intracellular granules. Finally, this cell line had never been grown in serum free medium (SFM) or grown with agitation. Each of these issues was addressed in this example.

In summary, lentiviral transductions of CHO and MST cells were performed to modify the HS biosynthetic pathway to produce anticoagulant rHS. Several genes, alone and in combination, were tested for their ability to produce the heparin-like product. No engineered CHO cells produced rHS with sufficient anti-Factor Xa (FXa) activity, a measure of inhibition of the coagulation cascade. Transduced MST cells, however, had much higher anti-FXa activity and it was found that at least one MST cell line produced rHS with anti-FXa activity, anti-FIla activity, protamine reversibility and in vivo potency equal to pharmaceutical heparin. These results show that a rHS product has been produced with quality equivalent to pharmaceutical heparin.

Two genetic approaches were pursued to reduce binding of the rHS to platelet factor 4 (PF4), which should decrease the risk of heparin-induced thrombocytopenia (HIT). Genetic elimination of 2-O-sulfate in MST cells reduced PF4 binding but also reduced the anti-FXa inhibition activity to unacceptable levels. Overexpression of Sulf2 yielded colonies with reduced PF4 binding that retained the majority of their anti-FXa activity. Thus, overexpressing Sulf2 would very likely yield a heparin replacement with reduced risk of HIT.

MST cells were successfully transitioned to SFM in shaker flask culture. Growth in SFM improved cell growth, increased rHS yield and maintained high anti-FXa activity. These findings suggest that rHS can be produced from MST cells in a biotechnology industry setting. Currently production of rHS in SFM grown in shaker flasks is 8 mg/L culture volume. To further improve the yield, xylosides were added to prime rHS synthesis, which increased the yield by about 80 percent. This indicates that production may be limited by core protein production or by the xylosyltransferase that initiates synthesis on the core protein.

Eliminating CS in MST Cells

To eliminate production of CS, the biosynthetic enzymes responsible for production of CS in MST cells (ChSy1, CSGalNAcT1 and CSGalNAcT2) were knocked out using CRISPR/Cas9. CS production was previously eliminated in CHO-S cells using a similar approach, to produce the ChA27 cell line. Pairs of sgRNAs (Synthego) were used strategically to excise a large piece of DNA to inactivate the target genes. PCR was used to verify that all three genes had been mutated in a population of cells.

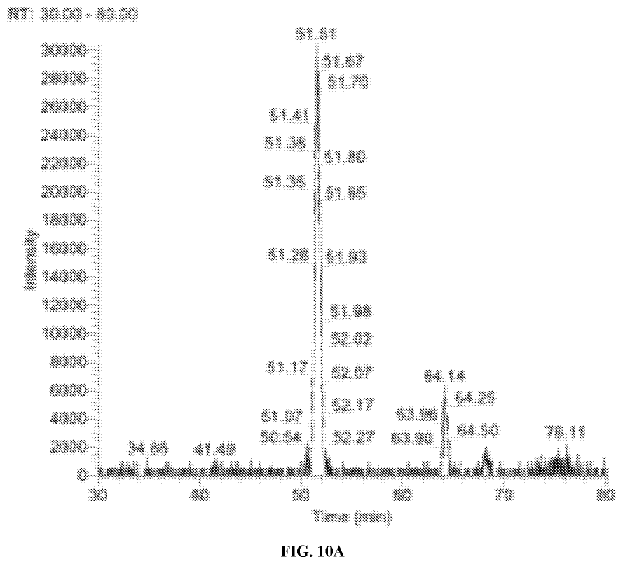

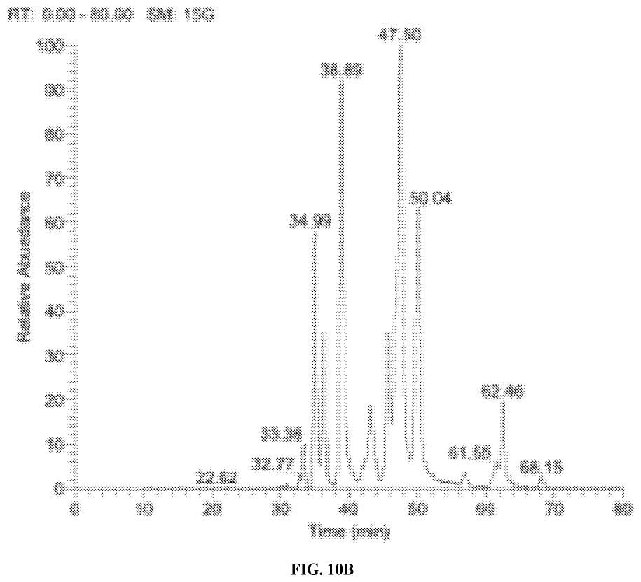

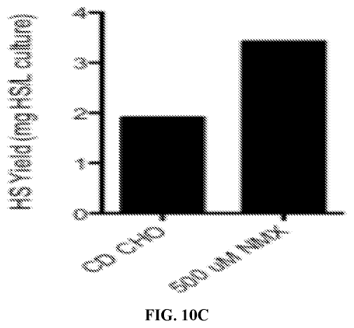

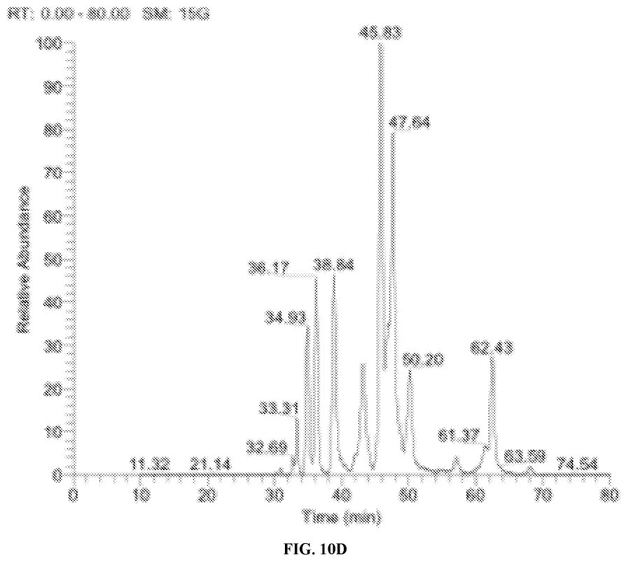

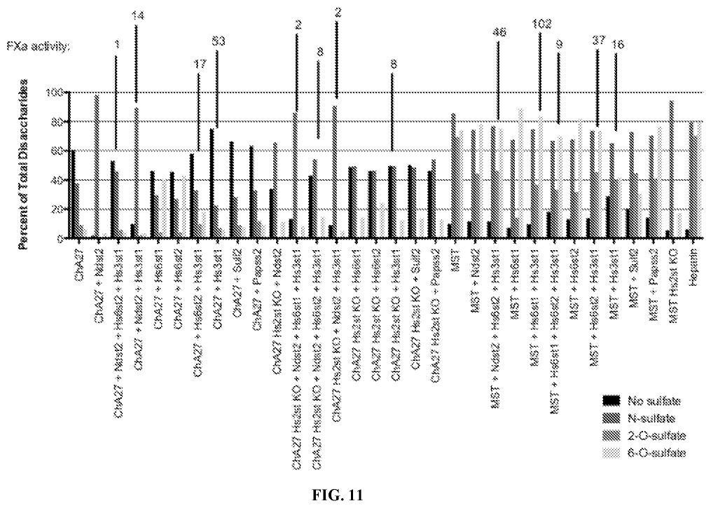

From 160 single cell colonies, seven were found with all three genes mutated. HS and CS were purified from the triple mutants and from a wildtype control cell line. Colony MST17B10 produced the highest HS yield (0.73 mg HS/L of DMEM/F12+15% FBS medium) and LCMS analysis verified that CS synthesis had been eliminated (FIG. 10). MST17B10 was used as the parental cell line for subsequent modification of the HS biosynthetic pathway.

Engineering Cells to Increase rHS Anticoagulant Potency

The preliminary RNAseq expression data, HS composition data and literature references indicated that HS sulfotransferases Ndst2, Hs6st2, and Hs3st1 were limiting and therefore candidates for overexpression in CHO cells. A similar analysis of MST cells indicated that Hs6st1 or Hs6st2 should be overexpressed along with Hs3st1. cDNAs for each of these genes were purchased (GenScript) and cloned into a lentiviral plasmids.

Spinoculation was used to transduce the cells under optimal conditions determined using a GFP inducible lentivirus detectable by flow cytometry. CHO and MST cells were then transduced with single or mixed lentiviral vectors. A method was developed to determine HS composition by liquid chromatography on a Waters Ultra High Pressure Liquid Chromatograph (UPLC). Briefly, purified rHS was digested exhaustively using heparin lyases, tagged with 2-aminoacridone and the disaccharides quantified by UPLC against standards of known mass.

For CHO cells, UPLC analysis showed the expected changes in composition based on the known activities of the transduced genes (FIG. 11). Specifically, overexpression of Hs6sts increased the prevalence of 6-O-sulfate. Overexpression of Ndst2 increased the abundance of N-sulfate at the expense of sulfate groups in other positions, which is consistent with previous reports. In MST cells, the effect of overexpression was not expected since these cells already have high levels of sulfate in each position although the abundance of 2-O-sulfation was reduced by overexpression of some other sulfotransferases. It is noted that overexpression of biosynthetic genes may have altered the sulfation patterns even though the overall abundance of sulfation at a certain position was unchanged.

Factor Xa Inhibition

FXa inhibition activity was determined for purified rHS from mixed cell populations (FIG. 11). The CHO+Hs3st1 transfected cell population had anti-FXa activity of 53 U/mg. All other CHO cell derived populations had activity lower than 20 U/mg. The MST population with the highest activity was MST+Hs6st1+Hs3st1 with 102 U/mg. It is emphasized that these compositions and FXa activity assay results were generated using rHS from transduced populations, which contain cells with different transgene copy numbers and insertion positions. Therefore, it was expected to find significant cell-to-cell variability in the transduced populations with rHS from some cells having much higher anti-FXa activity than the mean.

The mixed populations that had the highest anti-FXa activity were selected for limiting dilution cloning to create single cell colonies. These included CHO+Hs3st1, MST+Ndst2+Hs6st2+Hs3st1, MST+Hs6st2+Hs3st1, MST+Hs6st1+Hs3st1 and MST+Hs3st1. Two methods were developed to screen colonies.

In the first method, PCR primers were designed that were complementary to regions of the lentiviral plasmid that flanked the gene of interest. In this way, the same PCR reaction could be used to detect any of the transgenes that were inserted into the same viral vector. Since the cDNAs for these genes were all different sizes, the specific genes could be identified by the size of the PCR product on a gel.

Second, a streamlined purification method was developed that allowed for purification of rHS and assay anti-FXa activity from a 6-well dish in just a few hours. This shortens the typical HS purification process that normally requires 2-3 days. Briefly, the cells were grown in 6-well plates for 5 days. The cells and medium were lysed and protease digested before purification of the HS on DEAE-Sephacel. The FXa activity assay was adapted to directly analyze the column eluate to identify the wells with the highest anti-FXa activity.

PCR was employed to screen colonies from 96-well plates. Lentiviral transduction with a single gene typically resulted in 70-90 percent efficiency of transduction. The level of efficiency/gene was lower when multiple genes were transduced simultaneously where the frequency of colonies positive for three genes together could be as little at 10 percent. In this case, the PCR screen allowed us to rapidly eliminate untransfected colonies and move the positive colonies forward to the second screening method.

TABLE 6
Anti-FXa Activity of Single Cell Colonies

| | No. | Average anti- | Highest anti- |
|---|---|---|---|
| | Colonies | FXa activity | FXa activity |
| Genotype | Screened | per well (mU) | per well (mU) |
| MST + Hs3st1 | 181 | 413 | 1075 |
| MST + Hs3st1 + | 182 | 522 | 950 |
| Hs6st1 | | | |
| MST + Hs3st1 + | 27 | 160 | 750 |
| Hs6st2 | | | |
| MST + Hs3st1 + | 17 | 154 | 405 |
| Hs6st1 + Ndst2 | | | |
| MST + Hs3st1 + | 10 | 18 | 66 |
| Hs6st1 + Hs6st2 | | | |

Anti-FXa activity in the rHS from the CHO colonies was consistently low regardless of which genes were transduced (17.6 to 47.3 mU of activity per well). Therefore, MST cells were the focus as the rHS from these colonies appeared much more active (see Table 6).

Among the five genotypes, rHS from MST+Hs3st1 and MST+Hs3st1+Hs6st1 had the highest anti-FXa activities and the top colonies were grown in 30 mL shaker cultures to produce material for more thorough analyses. rHS was purified and quantified using the carbazole assay. The anti-FXa specific activity was assayed and the disaccharide composition was determined. To enable more extensive analyses on a clone with high anti-FXa activity (MST+Hs3st1+Hs6st1 38 or “MST38”), this colony was expanded to generate a 7 L culture. The cell pellet and conditioned medium were harvested separately and rHS was purified from each. 8.8 mg of rHS was purified from the cell pellet and 2.3 mg of rHS from the conditioned medium.

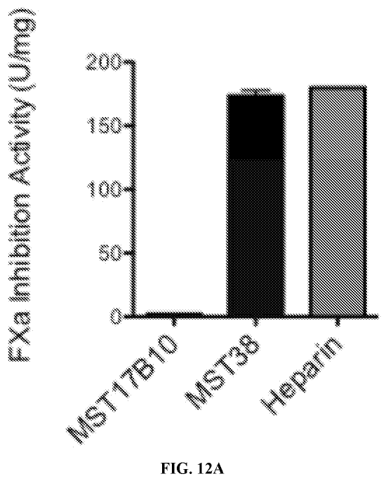

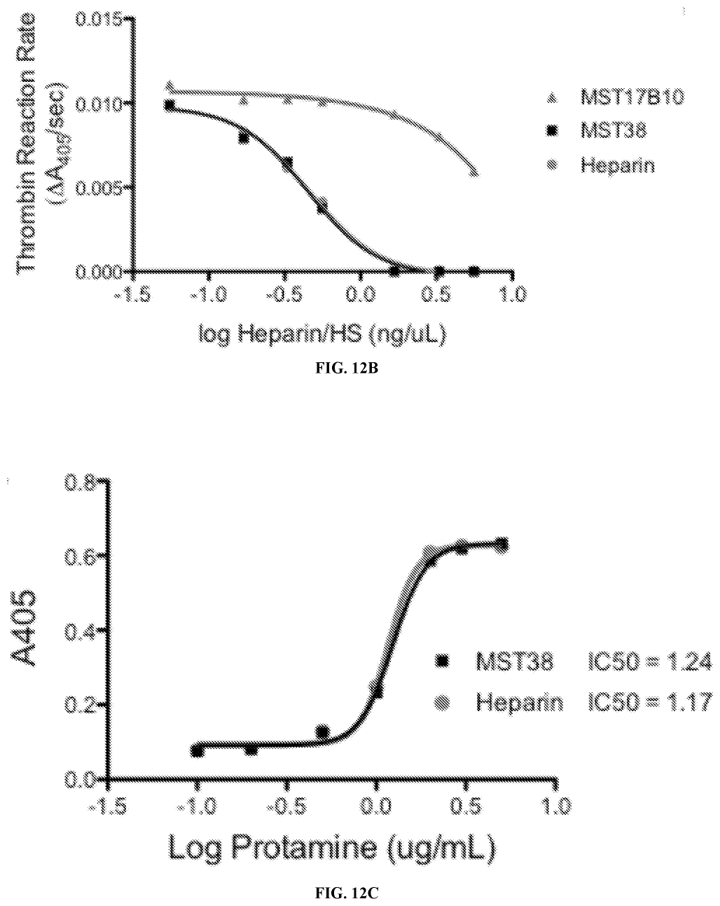

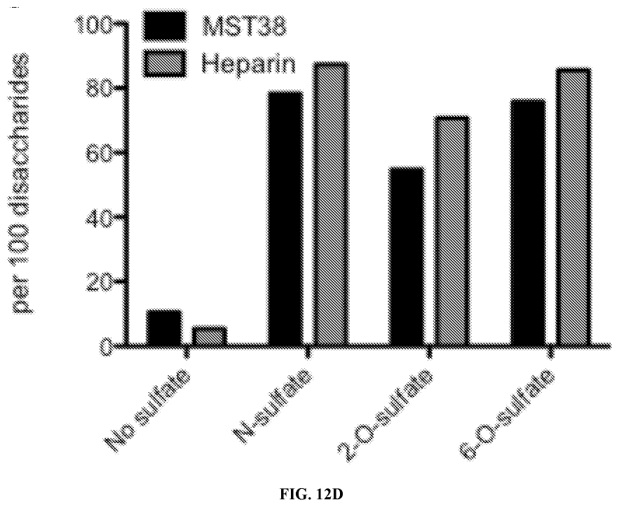

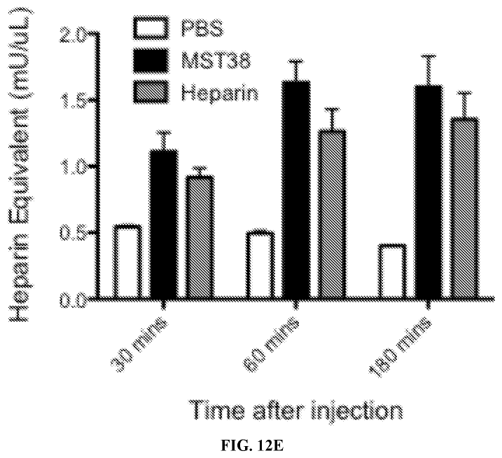

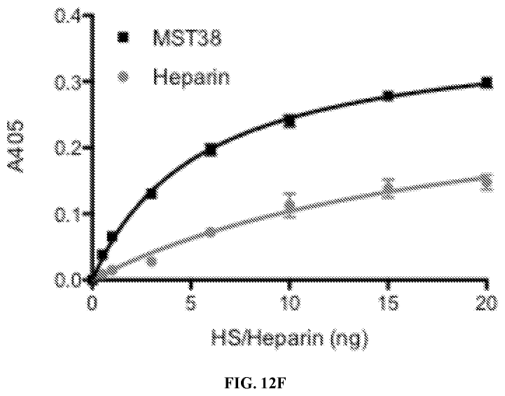

Analysis of activity showed that the cellular material had heparin-like anti-FXa specific activity of 172 U/mg (pharmaceutical heparin=180 U/mg) (FIG. 12A). The secreted material had lower specific activity (66 U/mg). Anti-FIIa activity in the cellular material was indistinguishable from pharmaceutical unfractionated heparin (FIG. 12B) in this assay. Importantly, protamine is used routinely in the clinic to reverse the anticoagulant effects of heparin for procedures like cardiopulmonary bypass surgery. The anti-FXa activity of MST38 rHS was completely neutralized by protamine, which was also indistinguishable from heparin (FIG. 12C). Thus, MST38 rHS was heparin-like in terms of its ability to inhibit enzymes in the coagulation cascade and its reversibility with protamine.

The disaccharide composition of MST38 rHS was determined and compared it to pharmaceutical heparin. It was found that MST38 rHS carried 209 sulfates per 100 disaccharides, which is lower than 244 sulfates per 100 disaccharides in pharmaceutical heparin. Overall lower sulfate content was attributed to an equal reduction of sulfation at each position in the rHS (FIG. 12D).

To test the activity of this material in vivo, MST38 rHS and pharmaceutical heparin were both injected subcutaneously into C57B16 mice (n=4, 3 mg/kg). Other mice were injected with vehicle (PBS) control. Blood samples were taken by tail bleed at 30 minutes, 60 minutes and 180 minutes after the injection. Assays of the plasma samples showed that the anti-FXa activity in plasma for mice treated with both pharmaceutical heparin and MST38 rHS was elevated above the PBS control at all time points. The potency of MST38 rHS trended higher than pharmaceutical heparin although this effect was not statistically significant. (FIG. 12E).

In summary, extensive genetic engineering and cell line screening was performed to identify several colonies that produce rHS with anti-FXa activity comparable to that of pharmaceutical heparin. rHS from MST38 had anti-FIla activity and reversibility by protamine equal to heparin. This material had lower sulfate content than pharmaceutical heparin but was also equally effective at inhibiting FXa activity in mouse plasma after a subcutaneous injection.

Engineering Cells to Reduce the Risk of HIT

Heparin induced thrombocytopenia is an immune response to heparin bound to PF4 and is the most common side effect of treatment with heparin. The immune reaction manifests clinically as a sharp drop in platelet count and, in some cases, venous thromboembolism. The reaction can be life threatening. Producing a heparin substitute from genetically engineered cells creates an opportunity to modify the biosynthetic pathway to limit PF4 binding.

An assay was developed to measure PF4 binding to HS. Briefly, HS was immobilized in a 96-well plate and biotin-PF4 is incubated in the wells followed by detection using streptavidin-HRP and a chromogenic substrate. Using this assay, it was found that MST38 has higher PF4 binding than pharmaceutical heparin (FIG. 12F). Based on information in the literature, two approaches were tested to reduce PF4 binding to rHS.

First, it has been established that PF4 binding to HS is sensitive to the level of 2-O-sulfate. There is also evidence that 2-O-sulfate is dispensable for high affinity antithrombin binding, which is required for inhibition of FXa and FIIa. The strategy was to eliminate 2-O-sulfate by knocking out the HS 2-O-sulfotransferase (Hs2st) gene. Using the CRISPR/Cas9 strategy described above, Hs2st was targeted in MST cells and the knockout was verified by PCR. Hs2st knockout cells were then transduced by lentivirus to overexpress Hs3st1 and single cell colonies were produced from this modification by limiting dilution cloning. PCR analysis showed the presence of the Hs3st1 transgene and the mutation of endogenous Hs2st. The rHS from eight colonies was tested for anti-FXa activity. The activity of these colonies was low with an average anti-FXa activity of 185 mU per well. Since these activities were less than half the activity that was observed in MST+Hs3st1 and MST+Hs3st1+Hs6st1, it was concluded that this approach would not give high enough anti-FXa activity to justify further pursuit.

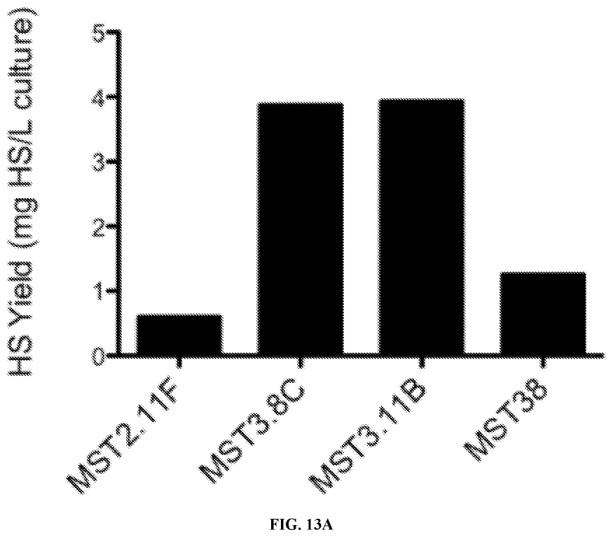

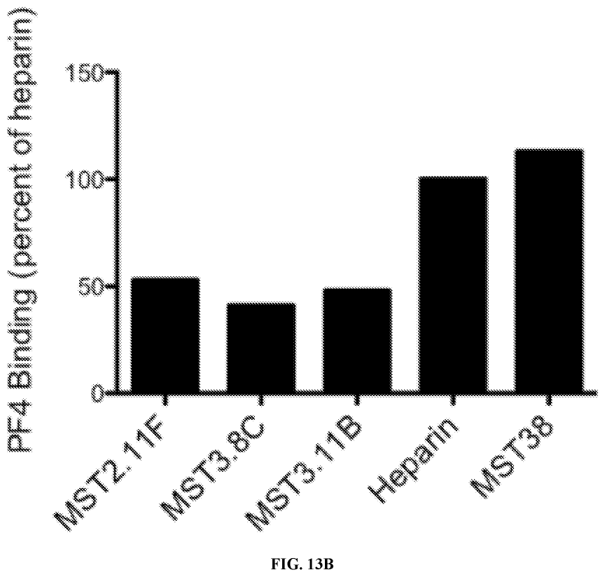

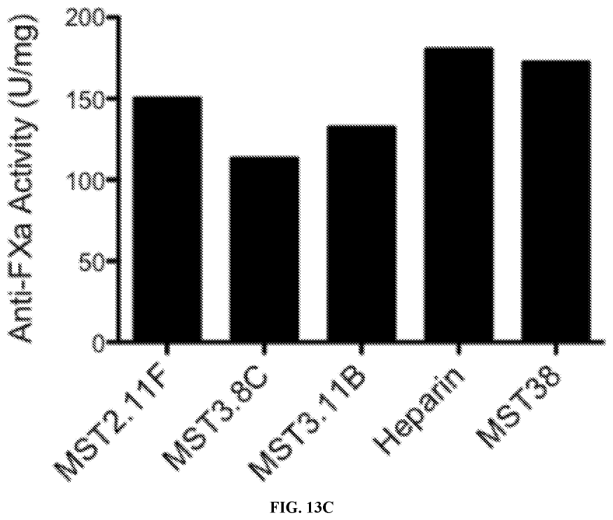

Second, it has been reported that Sulf2 (an extracellular HS 6-O-endosulfatase) selectively removes 6-O-sulfate groups that are required for PF4 binding without reducing anticoagulant potency. This report suggests that overexpression of Sulf2 in MST cells could reduce the risk of HIT. MST cells were transduced to overexpress Sulf2 and compositional analysis of rHS from the cells confirmed that the level of 6-O-sulfate was reduced (FIG. 11). Sulf2 was subsequently transduced into the MST+Hs3st1+Hs6st1 mixed population and limiting dilution cloning was performed to generate colonies from single cells. 285 colonies were screened by PCR to identify 41 that were positive for all three transgenes. The average anti-FXa activity of these colonies was 261 mU per well with the max activity of 855 mU per well. rHS was screened from these colonies for PF4 binding with comparison to MST38 rHS and pharmaceutical heparin. 13 of the 41 colonies had lower PF4 binding than pharmaceutical heparin. All of the colonies had lower binding than rHS from MST38. Ten colonies with the highest anti-FXa activity were transitioned into larger cultures for more extensive analysis. rHS purified from these cells had lower PF4 binding than pharmaceutical heparin and variable anti-FXa activity and yield. Two colonies in particular had low PF4 binding, high yield and high anti-FXa activity (FIG. 13A, FIG. 13B, FIG. 13C).

These results show that overexpression of Sulf2 is a promising strategy to limit PF4 binding in rHS while maintaining high anti-FXa activity. Additional screening likely yields a cell line with high anti-FXa activity and low PF4 binding that also has higher levels of production. Sulf2 is overexpressed in the final cell line to reduce PF4 binding.

Increase rHS production by optimizing growth conditions and medium formulation Adapting MST cells to suspension culture and serum free medium (SFM)

MST cells are traditionally grown in static suspension culture in DMEM/F12+15% FBS. Typical bioprocess methods use stir tanks or wave bags with suspension cells in SFM. MST cells growing in static culture were transferred into shaker flasks and found that the MST cells grew well with shaking.

MST cells were first tested in CD CHO medium (Gibco). At the beginning of this study, the MST38 cells had not been produced as yet, so the MST17B10 cells were tested in these experiments. To explore other media formulations, HyClone medium ActiPro, SFM4MAb and CDM4NSO were tried. The CHO-S variant (ChA27) was also tested in these media. Addition of even trace amounts of FBS in CD CHO or ActiPro media prevented MST cell growth, however the cells adapted to each of the SFM through a direct switch to the new medium. Cell growth lagged initially and then resumed its normal rate after a few days.

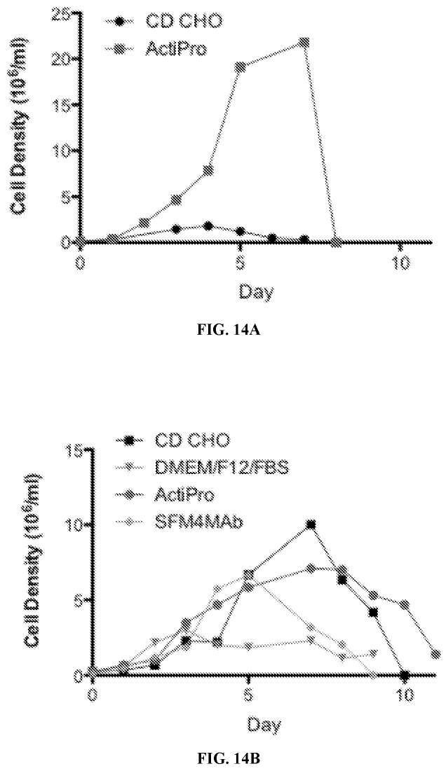

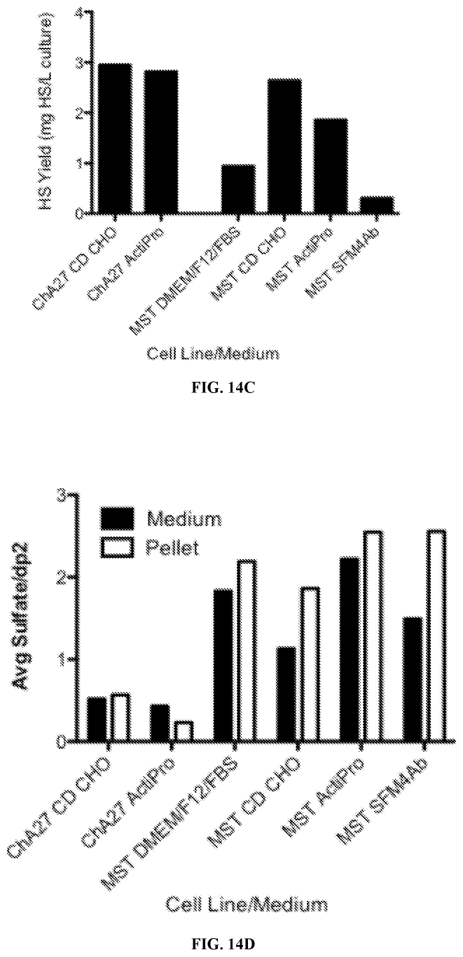

The growth characteristics and rHS production were investigated in each of the media. Adapted cells were seeded at 0.2×10\hat{ }6 cells/mL. Cell number and viability were counted daily. On day 10, total rHS (cellular and secreted) was purified from the cultures, quantified and characterized for disaccharide composition. For ChA27 cells, it was found that ActiPro supported about 20-fold higher integrated viable cell density (IVCD) than CD CHO medium (FIG. 14A). For MST17B10 cells, the IVCD was similar between CD CHO and ActiPro medium, both of which were about 2-fold higher than DMEM/F12+15% FBS and SFM4MAb (FIG. 14B). Despite having much higher IVCD in ActiPro, ChA27 cells produced roughly equivalent amounts of rHS (FIG. 14C). CD CHO resulted in the highest rHS yield for MST cells, which was about 30% higher than ActiPro and about 150% higher than DMEM/F12+FBS (FIG. 14C). While the level of sulfation in the rHS from ChA27 cells was similar between CD CHO and ActiPro, there were differences in MST cells where ActiPro resulted in the highest sulfation levels (FIG. 14D).

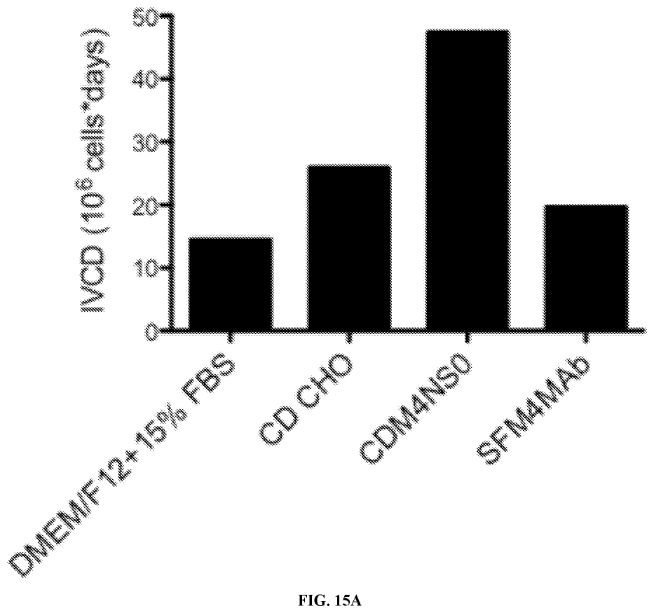

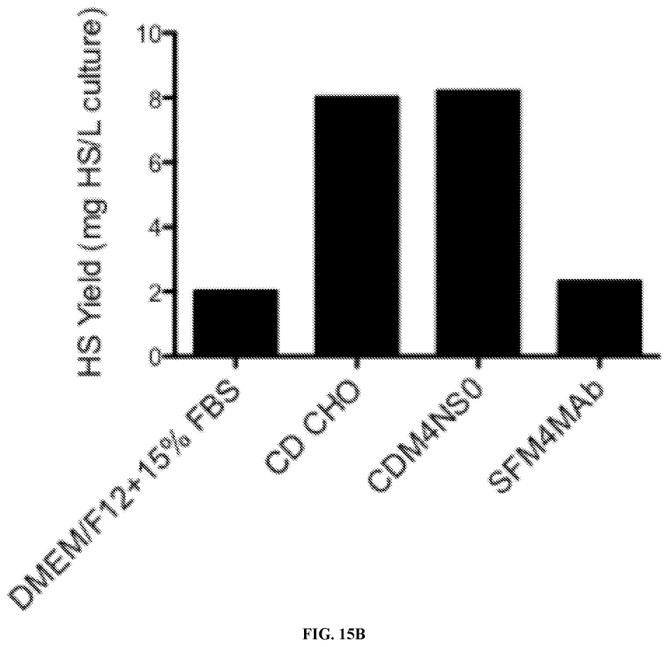

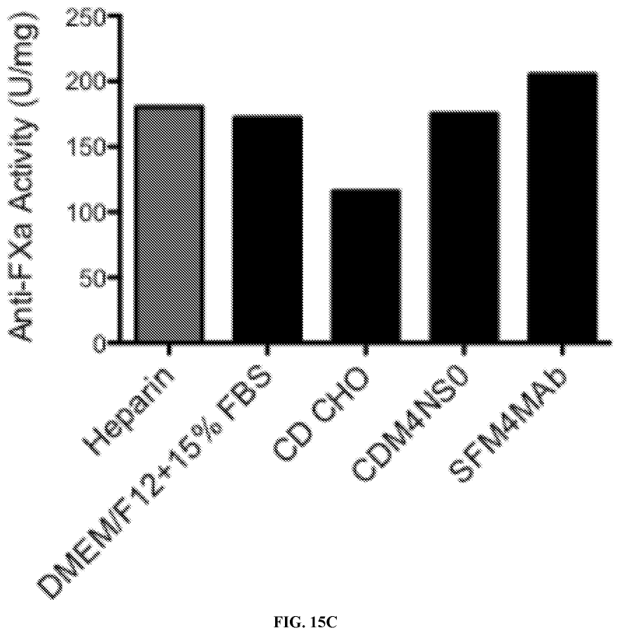

To investigate production of anticoagulant rHS in SFM, MST38 cells were adapted to the SFM and characterized the purified rHS. It was found that MST38 cells had better growth in SFM (FIG. 15A). The rHS yields were higher for CD CHO and CDM4NS0 medium than SFM4MAb and medium with serum (FIG. 15B). Furthermore, the CDM4NS0 and SFM4MAb both supported production of rHS with high anti-FXa activity (FIG. 15C). CDM4NS0 is a particularly promising starting point for future media formulation based on its high yield and high activity.

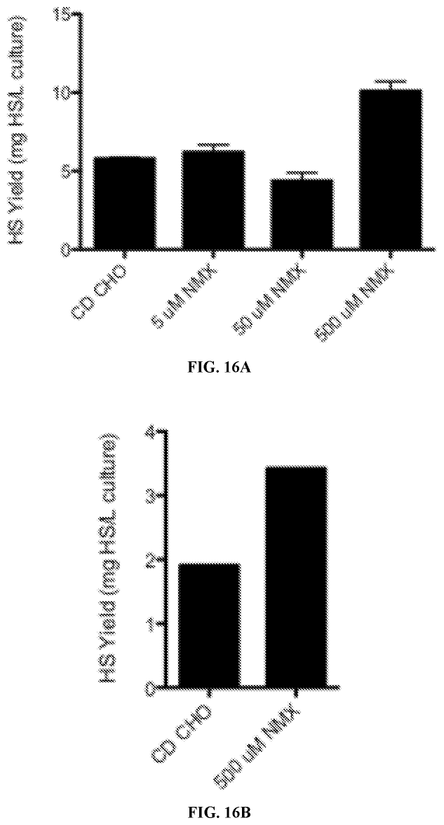

With respect to improving rHS yield from the cells, supplementation of the medium with xylosides was tested. Xylosides consist of xylose coupled to an aglycone that serves as a non-protein primer for HS synthesis. Inclusion of 500 μM xyloside with either ChA27 or MST17B10 cells increased the yield of glycosaminoglycans by ˜80 percent (FIG. 16A, FIG. 16B). This finding suggests that xylosides could be used to enhance production or that core protein synthesis or HS initiation by xylosyltransferase could be limiting production of rHS. Experiments with overexpression of serglycin core protein also are underway.

Hollow Fiber Bioreactor

Hollow fiber bioreactors (HFB) are an alternative to stir-tank bioreactors and have some advantages for culturing cells. In HFBs cells are grown on the outside of hollow fibers that create a semi-permeable barrier between the compartment where the cells are growing (cartridge) and the growth medium that flows through the inside of the hollow fibers. Nutrients are delivered to the cells through the porous fibers but the secreted macromolecules (proteins and polysaccharides) are retained in the extra-capillary space where they can accumulate to concentrations 100X higher than in other cell culture formats facilitating purification from much smaller volumes. Small diameter fibers provide large surface areas for nutrient and waste exchange enabling the cells to grow at over 10\hat{ }8 cells/ml achieving tissue-like densities. These densities could influence production but also enhance performance in SFM, an important consideration for producing high specific activity rHS from MST cells. One disadvantage is that the current cartridge volumes limit production to 10 mg to 1 g quantities. However, FiberCell Systems has developed a large-scale system using a new generation of high flux fibers in a 1 L cartridge that has the potential to replace 10,000 L stirred tank reactors for mammalian production over a 100-day period of culture. The system features an environmental enclosure that can be fitted with HEPA filters to create a Class 100 clean room environment for cGMP production as well as sensors for pH, dissolved oxygen and glucose monitoring.

MST cells were tested in these bioreactors to increase the concentration of the product and streamline production. These experiments tested the cell line MST10H which produces rHS with detectable anticoagulant activity. Two HFB C2008 20 ml cartridges (FiberCell Systems®) with 5 kDa 50% MWCO fibers, were inoculated with MST10H cells as specified by the manufacturer. Cartridges were maintained in DMEM/F12+15% FBS with medium changes and glucose addition as needed. Cells and media were harvested at various times. rHS from cells and medium were purified separately and analyzed for quantity using the carbazole assay and for activity using the FXa assay (Table 7).

Experiment 1: MST10H cells and media were harvested at various times following establishment of the cells in the bioreactor cartridges and establishment of robust glucose consumption. The MST10H cells showed a range of yields and anti-FXa activity in DMEM/F12+15% FBS, some of which were higher than were observed in rHS from shaker flasks. Depletion of serum from the cartridge that occurred while harvesting cells may have contributed to the reduction in anti-FXa activity that was observed in later harvests. The variation in HS/CS production may be due to different culture durations and glucose feeds.

Experiment 2: In the second set of experiments, the two cartridges were synchronized by matching the initial MST10H cell densities. Glucose was added one time when the level dropped to 150 mg/dL (50% reduction) and afterwards cells were harvested when glucose levels dropped below 100 mg/dL. Two consecutive cell harvests were performed one week apart on cells grown continually on the same media to determine the total production capacity ofthe bioreactor/liter of medium. Total rHS production from a 500 ml bottle of DMEM/F12+15% FBS was 0.94 mg or 1.88 mg/L with anti-FXa activity measuring 109 and 148 U/mg from the two harvests. These values are higher than obtained from MST10H cells in shaker flasks.

CDM-HD (FiberCell Systems) is serum replacement supplement created specifically for use in the HFBs. In the DMEM/F12+10% CDM-HD medium, the rHS production capacity was higher, reaching 3.74 mg/L. The anti-FXa activity in CDM-HD however, was lower indicating that additional components may be needed to achieve the activity seen with serum. rHS from the medium was not analyzed as previous experiments showed lower anticoagulant activity in secreted rHS. These pilot experiments provide a foundation for subsequent experiments with the newly developed cell lines (e.g. MST38). As shown above, production of rHS in MST38 cells is increased in various SFM from GE Healthcare while maintaining potent anticoagulant activity. Therefore, various media formulations can be tested if hollow fiber bioreactors are determined to be the best format for production.

TABLE 7
rHS production from MST10H cells in hollow fiber bioreactors

| Experiment/ | Sample | | Medium | GAG | Anti-Fxa |
|---|---|---|---|---|---|
| Sample # | Type | Medium Type | Volume | (mg) | (U/mg) |
| Experiment 1/ | Cells | DMEM/F12 | N/A | 0.378 | 157 |
| Sample 1 | | 15% FBS | | | |
| Experiment 1/ | Medium | DMEM/F12 | N/A | 0.935 | 2 |
| Sample 1 | | 15% FBS | | | |
| Experiment 1/ | Cells | DMEM/F12 | N/A | 0.646 | 73 |
| Sample 2 | | 15% FBS | | | |
| Experiment 1/ | Medium | DMEM/F12 | N/A | 0.390 | ND |
| Sample 2 | | 15% FBS | | | |
| Experiment 1/ | Cells | DMEM/F12 | N/A | 0.908 | 85 |
| Sample 3 | | 15% FBS | | | |
| Experiment 1/ | Medium | DMEM/F12 | N/A | 0.700 | 15 |
| Sample 3 | | 15% FBS | | | |
| Experiment 1/ | Cells | DMEM/F12 | N/A | 1.405 | 33 |
| Sample 4 | | 15% FBS | | | |
| Experiment 1/ | Medium | DMEM/F12 | N/A | 0.385 | 12 |
| Sample 4 | | 15% FBS | | | |
| Experiment 2/ | Cells | DMEM/F12 | 500 ml | 0.68 | 109 |
| Sample 1 | | 15% FBS | | | |
| Experiment 2/ | Cells | DMEM/F12 | 500 ml | 0.26 | 148 |
| Sample 2 | | 15% FBS | | | |
| Experiment 2/ | Cells | CDM-HD | 500 ml | 1.45 | 72 |
| Sample 3 | | | | | |
| Experiment 2/ | Cells | CDM-HD | 500 ml | 0.42 | 86 |
| Sample 4 | | | | | |

Conclusion

A mammalian cell line has been developed that produces rHS with biochemical properties and anticoagulant potency equal to pharmaceutical heparin. Having identified a recombinant source of this essential anticoagulant is a major milestone. While MST cells have never before been used for bio-manufacturing, it has been shown that they grow in shaker culture with SFM, two major prerequisites for scalable GMP production, and produce high quality material in this setting. It has also been demonstrated that reduction of HIT may be possible by overexpressing Sulf2.

### Example 11: Eliminating Rate-Limiting Steps and Degradation Pathways in HS Metabolism

Production of rHS with properties comparable to pharmaceutical heparin is modified to increase the amount of rHS produced by the cells. This is accomplished by overexpressing one or more enzymes involved in HS biosynthesis. These selected enzymes are listed in Table 8.

TABLE 8
Proteins involved in HS biosynthesis for overexpression

| | | Accession | |
|---|---|---|---|
| Protein Name | Gene | Number | Purpose |
| Syndecan 1 ectodomain | SDC1 | NM_002997.5 | Increase attachment sites and secretion |
| Glypican 1 ectodomain | GPC1 | NM_002081.3 | |
| Xylosyltransferase 1 | XYLT1 | NM_022166.4 | Biosynthesis initiation |
| Xylosyltransferase 2 | XYLT2 | NM_022167.4 | |
| UDP-glucuronate | UXS1 | NM_025076.5 | Increase pool of UDP-Xyl |
| decarboxylase 1 | | | |
| UDP-glucose 6- | UGDH | NM_003359.4 | Increase pool of UDP-GlcA |
| dehydrogenase | | | |
| N-acetyglucosamine | NAGK | NM_017567.6 | Increase pool of GlcNAc-6-P |
| kinase | | | |
| UDP-galactose-4- | GALE | NM_000403.4 | Increase pool of UDP-GlcNAc and |
| epimerase | | | UDP-Gal |
| Solube carrier family | SLC35D1 | NM_015139.3 | Increase pool of UDP-GlcA in Golgi |
| 35 member D1 | | | |

Many genes are involved in the HS biosynthetic pathway and metabolism of nucleotide sugars. This genetic engineering approach is guided by preliminary data and the known roles of these enzymes. It was shown that the use of xylosides as a culture medium supplement increased the production of rHS in MST17B10 cells by 80 percent (FIG. 16B). Since the xyloside bypasses the need for core protein and Xyl, one of those steps may be limiting in rHS production. Therefore, the ectodomains of two HS proteoglycan core proteins (Syndecan 1, Glypican 1) are overexpressed. Shedding of proteoglycan ectodomains has been shown to result in secretion of the HS rather than retention on the cell surface. Endogenous production of serglycin, which is the HS proteoglycan protein stored in granules, is also knocked out in an attempt to make MST cells secrete their rHS rather than store it in granules. A secreted rHS simplifies purification, leading to reduced production costs. The two xylosyltransferase genes (XYLT1/2) is also overexpressed because the xylosyltransferase step is sometimes limiting.

Quantification of intracellular pools of nucleotide sugars and their intermediates in MST38 cells is conducted to find limitations in availability of activated sugar precursors. It has been shown that UDP-Xyl levels are low in CHO cells whereas UDP-hexose and UDP-GlcA are abundant. UDP-glucuronate decarboxylase 1 (UXS1) is overexpressed to increase intracellular pools of UDP-Xyl and possibly boost polysaccharide chain initiation. The pools of other activated sugar precursors are manipulated by overexpressing UDP-glucose 6-dehydrogenase (UGDH), N-acetylglucosamine kinase (NAGK) and UDP-galactose-4-epimerase (GALE). In addition to providing for HS biosynthesis, UDP-GlcA is used in the cytosol for glucuronidation. This may deplete the pool available for rHS synthesis in the Golgi. To divert more UDP-GlcA to the Golgi for rHS production, the relevant transporter (SLC35D1) is overexpressed. UDP-GlcNAc and UDP-GlcA are used in the cytoplasm by some cells for synthesis of hyaluronan. It has been verified by RNAseq that the hyaluronan synthases are not expressed in MST cells. The chondroitin sulfate biosynthetic pathway is also eliminated, which would also compete for resources. These genetic modifications are coupled with supplementation of medium with sugars (GalNAc, GlcNAc, Gal) to boost the pools of nucleotide sugars.

To overexpress these enzymes, cDNAs for the genes of interest are cloned into a lentiviral expression plasmid (pHIV7-CMV-MCS). Purified lentivirus is produced for each gene. Lentiviral transduction is conducted using identified conditions that give >80% transduction efficiency in DMEM/F12+FBS, which was found to be sufficient to evaluate the effect of the transgene on the transduced population, even considering cell-to-cell heterogeneity arising from differences in copy number, genomic integration site and so forth. To increase rHS production, MST38 cells are transduced with each lentivirus individually. Transduction is performed with 10\hat{ }5 cells in 0.5 mL of DMEM/F12+15% FBS with MOIs of 0, 10, 30 and 100. An MOI of 100 was previously used to engineer MST cells for high anti-FXa activity. A modified spinoculation protocol is used to drive the virus and suspension cells together at the bottom of a conical tube. A mock transduction and GFP-expressing lentivirus serves as a control. PCR with primers specific to the regions of the lentiviral plasmid that flank the transgene is used to verify that transduction was successful.

Transduced cells are scaled up to 30 mL cultures in shaker flasks. After freezing down the transduced cells, the shaker flasks are reseeded with 0.2×10\hat{ }6 cells/mL in DMEM/F12+15% FBS and incubated for 7 days. The medium and the cells are collected separately. Cells are lysed by addition of 30 mL of low salt buffer (50 mM NaOAc, pH 6.0, 250 mM NaCl)+0.1% Triton X100 and 0.5 mg/mL of Pronase, shaking for one hour. To harvest secreted material, Triton X100 and Pronase are added directly to the conditioned medium. The rHS is purified on a 0.5 mL bed of DEAE-Sephacel (GE Healthcare) with on-column DNase

(Worthington) digestion to remove contaminating DNA. The rHS is eluted with high salt buffer (50 mM NaOAc, pH 6.0, 2 M NaCl) and desalted on a PD10 desalting column (GE Healthcare). The purified samples are then lyophilized to dryness and reconstituted in water. rHS mass is determined using the carbazole assay and anti-FXa activity is determined used the FXa assay.

When more than one gene that increases rHS yields in the first round of transductions, one transduced population is transduced with the other candidate gene(s) to combine the effects. The double transduced population is handled as described above to determine if the combination of transgenes has an additive or synergistic effect on rHS production. Additionally, genes that increase production are probably rate limiting metabolic steps. Once rates are no longer limiting at these steps, other points in the pathway may become rate limiting. Therefore, this approach is an iterative process where the best cell populations from the first round of transductions are transduced again with additional genes to try and overcome the new steps that become rate limiting.

Overexpressing these enzymes is expected to give at least 12-fold increase in rHS yield. Additional modifications include a number of other candidate genes involved in HS linker production and chain elongation (β4GALT7, β3GALT6, β3GAT3, EXTL3, EXT1, EXT2).

If, increased production outpaces the supply of sulfate donors for fully modified chains, PAPSS1/2 (sulfate donor synthases) and SLC35B2 (sulfate donor transporter to the Golgi) is overexpressed or the medium is supplemented to increase sulfate donor pools in the Golgi. The highest production is achieved by screening individual clones. Single cell colonies are isolated by limiting dilution cloning. Colonies are screened by one or more of three methods.

First, PCR with primers specific to the lentiviral plasmid to identify the colonies that have one or more transgenes incorporated in the genome. Multiple transgenes are differentiated by size on gels. This method is used as a first pass screen to eliminate untransduced colonies.

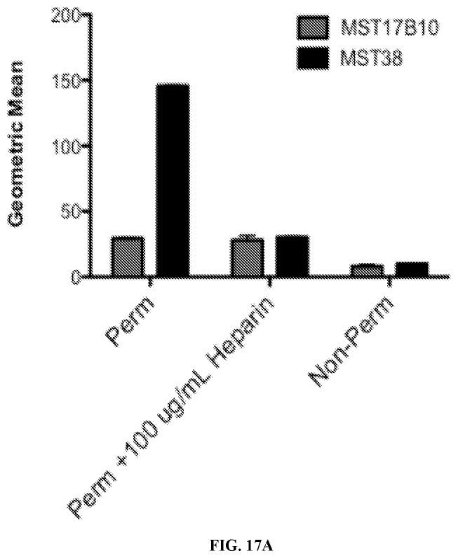

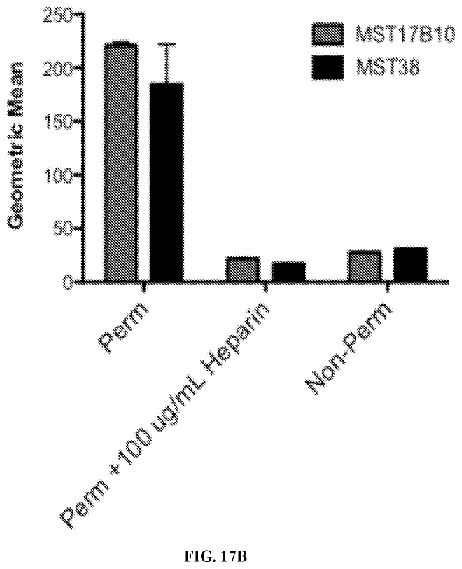

Second, Flow cytometry is used on colonies from a 96-well plate. Flow cytometry on cells in 96-well plates assesses the abundance of rHS in cells by detecting the levels of fluorophore-conjugated protein binding to the cellular rHS. Heparin binding proteins such as fibroblast growth factor 2 (FGF2) or antithrombin III (ATIII) are used to bind rHS in intracellular granules in permeabilized MST cells (Fixation/Permeabilization Kit, Beckton Dickinson) (FIG. 17A and FIG. 17B). This method facilitates screening hundreds of colonies per day with higher flow cytometry signals corresponding to greater abundance of rHS in the cell. With ATIII binding, this is also a measure of the abundance of the rHS anticoagulant motif. A secreted rHS is studied by screening cell supernatants using filter-based assays in which the rHS is immobilized to a filter and the fluorophore binding is detected using a fluorescent microplate reader.

Third, a streamlined rHS purification method to quickly assay anti-FXa activity. This method, involves growing the colony in a 6-well dish for 5 days and then releasing rHS into solution by adding Pronase and Triton X100 directly to the culture. The digest is then cleaned up by an abbreviated DEAE chromatography step. The anti-FXa activity assay is modified to accommodate the high salt concentration in elution buffer. This method provides a direct measurement of anti-FXa activity. Clone selection is based upon a combination of quality and quantity considerations.

HS degradative enzymes are targeted using CRISPR/Cas9. Briefly, MST cells are transfected with sgRNAs (Synthego) and recombinant Cas9 (Synthego). Pairs of sgRNAs against the genes are used to excise a large section from the gene. Success is verified by PCR. rHS from the population is then quantified to determine if yield has increased. Limiting dilution cloning is performed to isolate colonies with the highest rHS yield and quality.

### Example 12: Optimize Medium, Feeds, Supplements and Bioprocess Methods for Production of rHS in MST Cells

Because there is no established paradigm, the media requirements for production of rHS in MST must be established experimentally. In initial experiments the MST38 cells were adapted to four media: CD CHO (Gibco), ActiPro, SFM4MAb and CDM4NSO (GE) with improvement in IVCD, yield and anti-FXa activity (FIG. 15A, FIG. 15B, FIG. 15C). Medium formulation is improved for rHS production. MST38 cells are grown again in SFM4MAb, CDM4NS0 and ActiPro and cell number, viability and glucose concentration are determined daily. To identify the limitations of the basal medium and cell growth requirements, rHS quantity and quality and spent medium metabolite analyses are performed on days 3, 5, 7 and 10. This experiment is then repeated with the top media in combination with added glucose and HyClone Cell Boost feeds. From this data, custom media formulations and feeds are designed. Similar analyses in an iterative process continues to improve cell growth and production. A 10-fold increase in production was achieved from CHO cells by improving medium and feeds. All genetically modified lines are retested to confirm their growth and production in the optimized media.

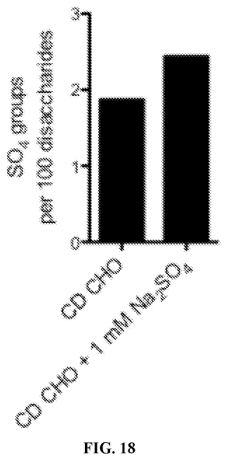

Production is further increased by the use of supplements specifically aimed at rHS. Supplementation with Gal feeds into the intracellular pools of UDP-Xyl, UDP-GlcA and UDP-Gal within just a few enzymatic steps. Supplementation with glucosamine, GlcNAc or GalNAc feeds into the pool of UDP-GlcNAc. Supplementation of culture medium with uridine, which has been shown to increase intracellular UDP-GlcNAc in CHO cells is tested. MST cells are cultured in SFM with different concentrations (1, 3, 10 mM) of each sugar or uridine. Cell growth characteristics and rHS quantity and quality are assessed. If increased biosynthesis results in undersulfated/underactive rHS, supplementing the medium with sodium sulfate is tested, which increased rHS sulfate content but slowed cell growth in preliminary experiments (FIG. 18). It is calculated that medium must have >4 mM available sulfate to support production of heparin-like rHS at 1 g/L. Providing this level of sulfate in the culture medium without excessively compromising cell growth is an important consideration in development of custom medium.

In conclusion, medium formulation and feeds are optimized for production of rHS in MST cells. Specific supplements and bioreactor process methods are explored to support rHS production. These strategies together with those described in Example 11 are expected to achieve production levels greater than 1 g/L.

### Example 13: Inhibition of Viral Infection

Heparin has been known for some time to inhibit attachment and integration of a number of viruses (Herpes, Influenza, human immunodeficiency virus, coronavirus) by blocking a viral envelope glycoprotein that binds to cell surface HS. Coronavirus entry into host cells is facilitated by the transmembrane spike glycoprotein that forms homotrimers protruding from the viral surface. Indeed, as the spike glycoprotein is exposed on the virus surface and is required for cell entry, it is the main target of neutralizing antibodies upon infection and is the focus of therapeutic vaccine design.

Work on coronaviruses from the SARS-CoV outbreak identified HS as the primary attachment factor on the surfaces of susceptible host cells. Another coronavirus strain (HCoV-NL63) that is endemic to humans and is one of the viruses responsible for the common cold also requires cell surface HS for cell adhesion. Most recently the spike protein of the SARS-CoV-2 was shown to bind HS, which induced conformational changes that may be necessary for activity. Furthermore, primary sequence analyses and modeling of the SARS-CoV-2 spike receptor binding domain shows potential heparin binding sites as patches of basic amino acids on the spike protein surface. These findings indicate that spike protein binding to cell surface HS may be an attachment strategy employed broadly by coronavirus strains as a precursor to viral invasion into the host cell.

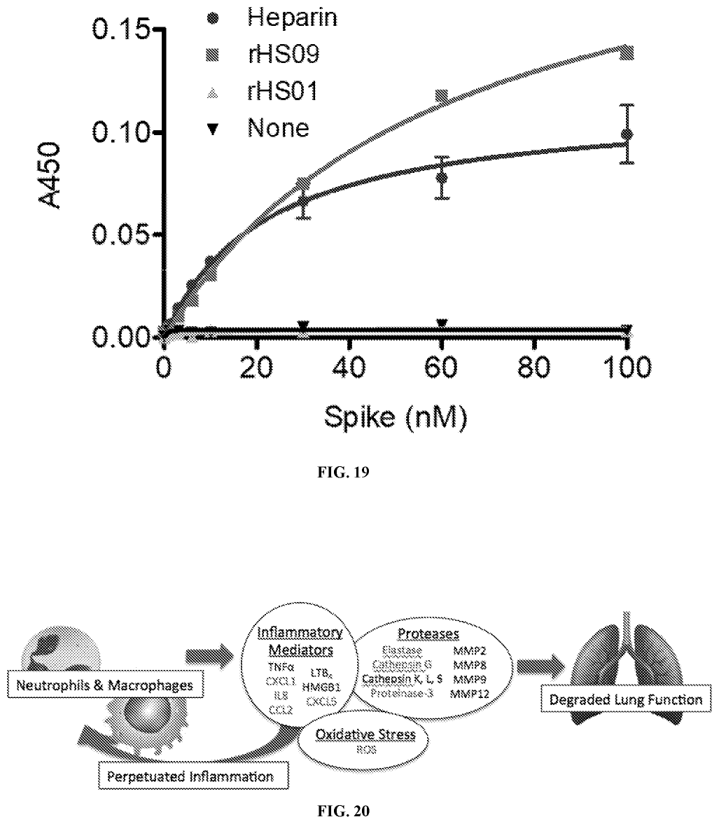

50 ng of heparin, 50 ng rHS01 (CHO) HS and 50 ng rHS09 (MST) HS was immobilized on a 96-well plate using 90% saturated ammonium sulfate. The wells were washed and blocked with PBST+0.1% BSA. Then, the wells were incubated with various concentrations of SARS-CoV-2 his-tagged spike protein extracellular domain (GenScript) followed by detection with anti-his-HRP antibody (GenScript). Binding was quantified by development of HRP substrate TMB Turbo (Pierce) and measurement at A450. While rHS01 failed to show any binding, rHS09 from MST cells supported binding at a level higher than heparin (FIG. 19).

### Example 14: Treatment of Chronic Obstructive Pulmonary Disease (COPD)

Chronic obstructive pulmonary disease (COPD) is a respiratory condition characterized by airflow limitation and difficulty breathing. It is thought that more than 5 percent of the US population is affected by COPD and it is responsible for more than 120,000 deaths annually in the US, making it the 4th highest cause of death. As a common and chronic condition, management of COPD is a large burden on the healthcare system costing an estimated $49 billion USD in 2020 and on the US economy at large with an estimated $3.9 billion USD in total absentee costs and 16.4 million work days lost in 2010.

About 80 percent of COPD cases result from long-term inhalation of noxious particles or gases, including cigarette smoke and environmental/occupational exposure to fumes and dusts. Other diseases or underlying genetic factors such as asthma and alpha-1 antitrypsin deficiency can also lead to the development of COPD. For COPD resulting from inhaled irritants, long-term exposure induces a chronic inflammatory response, which persists in the absence of the initial irritant. Pulmonary inflammation involves excessive inflammatory mediators, oxidative stress and unchecked elastolytic activity, which destroys elasticity and compromises the ability of small airways to remain open during expiration. Airflow is limited resulting in hyperinflation, reduced inspiratory capacity and gas exchange abnormalities. For progressing COPD, patients experience accelerated deterioration of lung function and acute exacerbations which are indicative of rapid decline and increased mortality.

COPD has no cure and the goal of routine treatment is to alleviate symptoms, limit the progression of the disease and reduce the number and frequency of acute exacerbations to extend the overall quality and length of life. Halting disease progression requires blocking chronic inflammation however anti-inflammatory steroids have limited benefit that must be weighed against their risk of adverse effects. Targeted therapies have shown limited benefit in treating COPD, likely because of the multi-faceted nature of the inflammation. The lack of a highly effective and well-tolerated anti-inflammatory agent underscores the importance of developing a new therapy to limit chronic inflammation and halt the progression of COPD.

Heparin has been used routinely as an anticoagulant in the clinic for more than 100 years with hundreds of thousands of doses administered daily in the US and the global heparin market currently exceeds $7 billion USD. In addition to preventing coagulation, an abundance of sulfate group negative charges on heparin results in binding to many additional proteins (including cytokines, enzymes and structural proteins) based on electrostatic interactions with basic amino acids. These interactions modify the activity of binding proteins in many ways including altered enzyme activity and cell signaling. Because of its many binding partners, many pharmacological properties have been observed in heparin. Among them is a modulatory effect on inflammation.

Heparin offers a distinct advantage over targeted therapies because it inhibits multiple targets among the three major facets of COPD—protease activity, oxidative stress and chemokines. Heparin has been used with benefit to pulmonary function in clinical trials for COPD, however the dose that can be administered is limited by anticoagulant activity and clinicians fear giving effective doses for risk of bleeding. To eliminate anticoagulant activity, heparin has been chemically modified but this drug had no efficacy in the clinic, likely because sulfate groups required to inhibit inflammation were also removed. Heparin is a highly sulfated form of heparan sulfate, a polysaccharide produced by all mammalian cells. Through genetic engineering of the heparan sulfate biosynthetic pathway in mammalian cells, recombinant heparan sulfates (rHSs) have been created with sulfate content similar to heparin but lacking specifically a rare sulfate group that is required for anticoagulation. These non-anticoagulant rHSs retain their capacity to bind to most heparin binding proteins, including inflammatory mediators of COPD. An rHS with anti-inflammatory properties equal to or superior to heparin with no anticoagulant potency is developed.

The molecular basis of COPD is complex involving multiple cytokines in what is an active area of research. COPD is characterized by chronic pulmonary inflammation with elevated numbers of macrophages, lymphocytes and neutrophils in the lungs. Macrophages and neutrophils release inflammatory mediators and reactive oxygen species (ROS) that perpetuate the cycle of inflammation and elastolytic proteases that degrade lung function (FIG. 20). First, monocytes are recruited to the lungs by CCL2 and CXCL1 where they differentiate into activated macrophages and release proteases and inflammatory mediators including TNFα, CXCL1, IL8, CCL2, LTB4, HMGB1 and reactive oxygen species (ROS). Second, neutrophils are recruited to the airways by CXCL1, CXCL5, IL8, LTB4 and HMGB1 where they release proteases including elastase, cathepsin G, proteinase-3, MMP9 and MMP12. Since multiple mediators are involved in COPD inflammation, in some cases, blocking a single factor will not have a positive clinical effect. This may explain why targeted therapies for COPD have had little or no effect in clinical trials. Heparin and heparin-like molecules offer a distinct opportunity to treat COPD because they bind to and inhibit the activity of multiple inflammatory targets including neutrophilic serine proteases, reactive oxygen species (ROS), and pro-inflammatory chemokines (FIG. 20).

Proteases—In healthy individuals, protease activity is balanced by endogenous protease inhibitors. In COPD, the proteases overwhelm inhibitors and degrade the alveolar walls. Heparin is a known inhibitor of neutrophil serine proteases elastase, cathepsin G and proteinase-3, which, together with MMPs, are responsible for the degradation of elastin, reducing lung elasticity, and generating chemotactic peptides that further drive inflammation.

Inflammatory mediators—All known chemokines bind to heparan sulfate. Binding to heparan sulfate in vivo establishes concentration gradients that facilitate directed chemotaxis for leukocytes and promotes oligomerization of chemokines. Biochemical studies have shown that heparan sulfate binding prevents primary drivers of pulmonary inflammation (CXCL1, CXCL5, IL8) from interacting with their receptors. Accordingly, addition of exogenous heparin in tissue culture was able to inhibit IL8-induced neutrophil chemotaxis.

Oxidative stress—Pulmonary oxidative stress occurs when the production of ROS exceeds intrinsic antioxidant capacity of the tissues and is a prominent feature in the lungs of patients with COPD. Activated neutrophils, macrophages and epithelial cells produce ROS that damage lipids, proteins and DNA. COPD patients have compromised response to oxidative stress and reduced activity of a tissue repair mechanism. Heparin's antioxidant effects have been appreciated for more than 30 years and include protection of endothelial cells from damage by toxic oxygen metabolites, enhancement of superoxide dismutase activity in vivo, and possibly acting as a free-radical sink. In a clinical trial, an IV dose of heparin reduced the ROS generation from leukocytes isolated from the blood.

Heparin has been tested for clinical efficacy in managing COPD. Injections of low molecular weight heparin during acute exacerbations of COPD significantly improved lung function and blood oxygenation but increased the patient's risk of bleeding. Inhaled heparin was safe and improved airway conductance, forced expiratory volume, exercise capacity, breathlessness, and ventilator free days in COPD patients but induced systemic anticoagulation at doses higher than 8 mg/kg. While the molecular mechanisms involved are under investigation, these clinical trials show a clear benefit derived from heparin in the management of COPD. Despite the success of these trials, heparin is not typically used to treat COPD because clinicians are afraid of the risk of bleeding. Indeed, the dose given to treat COPD is limited by the anticoagulant potency of the heparin, which in this application is an unwanted side effect. Use of heparin in the clinic can also cause heparin-induced thrombocytopenia, a potentially severe side effect resulting from an immune response to heparin/CXCL4 complexes.

Heparin is a linear polysaccharide comprised of uronic acid/N-acetylglucosamine repeats that are variably O-sulfated at the second carbon of uronic acids and the third and sixth carbons of N-acetylglucosamines. N-acetylglucosamine can also be converted to N-sulfoglucosamine, which predominates in heparin. Notably, pharmaceutical heparin has been chemically 2-O- and 3-O-desulfated (ODSH) to eliminate anticoagulant potency. Cantex Pharmaceuticals tested this product by infusion in patients experiencing exacerbation of COPD. While no safety issues were encountered, the clinical trial was terminated early because an interim analysis showed that there was no clinical benefit. As shown in this example, the chemical modification reduced the anti-inflammatory activities that were needed for efficacy.

Recombinant heparin-like products are produced to avoid the many problems with the current animal derived heparin supply chain. Pharmaceutical heparin is currently purified from pig intestines, primarily as a byproduct of pork production in China where hundreds of millions of pigs are slaughtered each year. Risks inherent in producing an essential drug from an animal population became apparent in 2007 when pig populations dropped suddenly because of a pig Blue Ear Disease outbreak in China. Crude heparin was adulterated with over-sulfated chondroitin sulfate to make up for the shortfall, causing hundreds of illnesses and at least 80 deaths in the US. An outbreak of African swine fever is currently spreading among the pig population in China where some reports indicate 40 percent of the swine population has been culled to limit the spread and at least one manufacturer has warned of upcoming heparin rationing

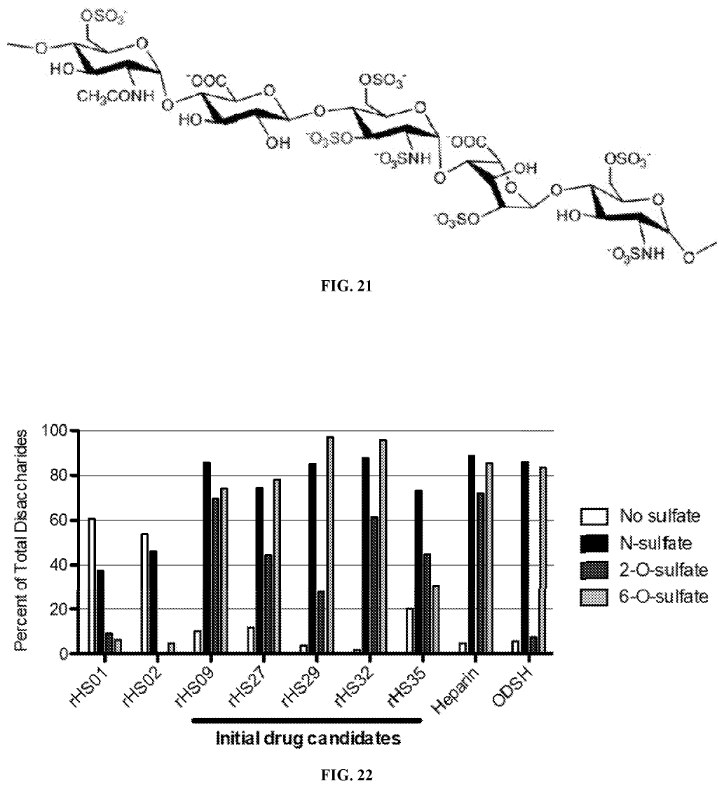

Heparin is produced specifically in mast cells and contains pentasaccharide domains (FIG. 21) with specific arrangements of sulfate groups that confer binding to antithrombin. Heparin binding increases the inhibitory activity of antithrombin against Factor II and Factor Xa in the coagulation cascade. Every mammalian cell has a biosynthetic pathway that produces heparan sulfate with generally lower sulfate content than heparin and little to no anticoagulant activity. To address the supply chain issues in animal-derived heparin, engineered mastocytoma (MST) cell lines have been developed to produce “recombinant heparin” with the same anticoagulant activity as porcine derived heparin. Importantly, reduced risk of HIT has been engineered into the recombinant heparin by reducing the binding of recombinant heparin to CXCL4.

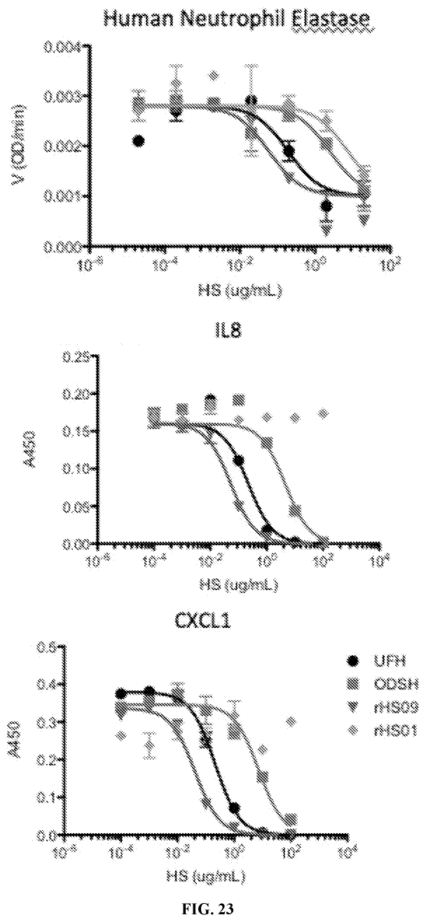

Recombinant heparan sulfate (rHS) products herein have been developed through engineering the mammalian biosynthetic pathway. The biosynthetic pathway consists of more than 20 enzymes (glycosyltransferases, sulfotransferases and an epimerase), each differentially expressed based on cell type and conditions. Heparan sulfate biosynthesis is not template driven and modifications do not go to completion resulting in great structural diversity. Heparan sulfate is typically characterized by degrading the chain to its disaccharide components and then measuring the abundance of each to determine a disaccharide composition. The frequency and arrangement of sulfate groups along the chain create binding sites for proteins. Proteins have different preferences for the frequency and spatial organization of the charges and preferences cannot be predicted without experimental data. Unlike anticoagulation, which requires a known pentasaccharide, the composition of heparan sulfate best suited for anti-inflammation cannot be predicted as it may involve inhibiting multiple components. However, a recombinant heparan sulfate (rHS09) has been produced that closely resembles pharmaceutical heparin in form and function but has no anticoagulant potency (FIG. 22). This is accomplished through targeted deletion of 3-O-sulfate, a rare but absolutely critical sulfate group for heparin's anticoagulant activity (63). Unlike ODSH, which has ˜90% of 2-O-sulfates chemically removed, this targeted approach leaves 2-O- and 6-O-sulfate intact. 3-O-sulfate appears to have limited involvement in heparin's anti-inflammatory properties.

To gauge the anti-inflammatory potential of rHSs compared to pharmaceutical heparin and ODSH, the rHSs were tested for neutrophil elastase inhibition and chemokine binding. Human neutrophil elastase was incubated with a chromogenic substrate and various concentrations of heparin, rHS01, rHS09 and ODSH. rHS01 and rHS09 are two of our rHSs where rHS01 has much lower sulfate content than heparin or rHS09. The velocity of the reaction was monitored over time at 405 nm. For chemokine assays, heparin was immobilized in microtiter wells. Recombinant IL8 or CXCL1 was incubated with different concentrations of each heparin/rHS in solution to compete for chemokine binding to the immobilized heparin. Bound chemokine was detected using biotinylated antibodies and streptavidin-HRP. Absorbance was measured at A450 and IC50 values were determined (FIG. 23). Notably, rHS09 had lower IC50 values than both ODSH and heparin for elastase and IL8/CXCL1. These results suggest that rHS09 has higher anti-inflammatory potency than ODSH and heparin.

One or more rHS types are identified from our library that are potent inhibitors against key chemokines and proteases. Disrupting the inflammatory cycle should inhibit tissue destruction and the deterioration of lung function in COPD patients. In some cases, this anti-inflammatory treatment is also effective in other lung diseases where pulmonary inflammation plays a negative role including cystic fibrosis, alpha-1 antitrypsin deficiency, and acute respiratory distress syndrome, such as that accompanying infection (e.g. COVID-19).

### Example 15: Engineering Heparan Sulfate Biosynthetic Pathway to Increase 2-O-Sulfate Content

By genetically engineering biosynthetic pathways, cell lines have been created that produce 26 varieties of non-anticoagulant heparan sulfate. Because their compositions vary, some may be better anti-inflammatory agents than heparin or rHS09. These properties must be determined empirically. Preliminary results indicate that 2-O-sulfate (FIG. 23) and 6-O-sulfate must be present for robust inhibition of elastase and binding to IL8 and CXCL1, although the optimal sulfate abundance is unknown. Given these requirements, five rHS candidates are selected from the non-anticoagulant library with high levels of 2-O- and/or 6-O-sulfate (rHS09, 27, 29, 32, 35). Additionally, CXCL4 binding should be limited to reduce the risk of HIT. CXCL4 binding also requires 2-O- and 6-O-sulfate so compositions with potent anti-inflammatory properties and low CXCL4 binding are identified to reduce the risk of HIT side effects. Different rHS compositions are produced by cell lines with different sulfotransferase expression patterns. rHS compositions in the library tend to have high levels of N-sulfate and 6-O-sulfate and lower levels of 2-O-sulfate. The binding properties of these rHS types are optimized by increasing 2-O-sulfate in these cell lines through over expression of Hs2st and then screening for the optimum combination of binding affinities.

Overexpression follows methods used to create the library of rHSs to date. The cDNA ORF for human Hs2st (NM_012262.4, GenScript) is cloned into pHIV-CMV-MCS. Lentivirus is used to transduce the selected cell lines using a spinoculation protocol with mock and GFP transductions used as controls. This protocol routinely gives >50% transduced cells with good cell viability. The success of transduction is measured using a PCR method with genomic DNA and primers that flank the multiple cloning site in the lentiviral plasmid. Single cell colonies are generated by limiting dilution cloning. Colonies from 96-well plates are screened by PCR for presence of the transgene. Twenty positive colonies are scaled up to shaker flask cultures in serum free production medium (Cytiva) for analytical heparan sulfate preps. Conditioned medium is filtered and passed over DEAE-Sephacel (Cytiva). The columns are washed with 50 mM NaOAc, pH 6, 250 mM NaCl and eluted in the same buffer with 2 M NaCl. Contaminating DNA and protein is enzymatically digested. The production of chondroitin sulfate is genetically eliminated. The heparan sulfate is purified again on DEAE-Sephacel, dialyzed to remove salt, lyophilized and then reconstituted in H₂O. Purity from DNA and protein are assessed by A260/280 and BCA protein assay (Thermo). The cell lines are characterized by the amount of heparan sulfate produced (carbazole assay) and UPLC disaccharide composition of the heparan sulfate following enzymatic digestion and fluorescent tagging of the disaccharide. Quantification is accomplished by comparison to disaccharide standards (Iduron) tagged in parallel and run on the UPLC in the same set.

Significant differences in production levels and disaccharide compositions are observed between single cell colonies from a single transduction. Cell lines with the highest heparan sulfate production levels and highest 2-O-sulfate content are selected for characterization against inflammatory targets. An additional 4-8 rHSs with 2-O-sulfate on ≥90% of disaccharides are selected. These new candidates are added to the 5 initial rHS candidates for further characterization.

### Example 16: Test Candidate Heparan Sulfates for Anti-Inflammatory Activity

The rHS with the greatest inhibitory potency against three major drivers of pulmonary inflammation in COPD will be identified—protease activity, oxidative stress and neutrophil/monocyte recruitment and activation. The rHS needed for these assays (10 mg each) is produced from 1 L of cell culture as described above. Since inhaled heparin has been shown to be beneficial for COPD patients, an rHS with in vitro potency equal to or superior to heparin is identified.

Proteases. The potency of rHSs to inhibit neutrophil elastase, cathepsin G and proteinase-3 using chromogenic substrates (0.75 mM S1384, 2 mM S7388, 5 mM M4765, respectively, Sigma) and enzymes purified from purulent human sputum (Elastin Products Company) is tested. Although the assay concentration for each enzyme has been reported (80 nM elastase; 20 nM cathepsin G; 100 nM proteinase-3) the enzymes are titered to establish the initial velocity. Each reaction is carried out in 125 mM HEPES, pH 7.4, 0.125% Triton X-100, 100 mM NaCl. Soluble rHSs (0-200 μg/mL) is added to the assay to test inhibitory activity. The extent of inhibition is determined as reduction of the initial velocity of the reaction.

Enzyme inhibition is also assessed by the top three rHS candidates in an assay with a physiological substrate (elastin-congo red, Sigma). In this assay, the insoluble macromolecular substrate releases congo red into solution when the elastin is cleaved. Enzyme activity is monitored by absorbance changes (A497) over several hours. This assay allows determination of whether protease inhibition is long-lived, which would be beneficial in treating patients. Protease (0.5 uM) is incubated with elastin-congo red (5 mg/mL) in reaction buffer above. 100 ug/mL of rHS is added to measure the extent of inhibition compared to no inhibitor. Absorbance is measured at multiple time points over several hours to determine the extent and longevity of inhibition.

Oxidative stress. The anti-oxidative capacity of rHSs is tested by monitoring the formation of a chromogenic radical cation (ABTS*+) from ABTS (2,2′-azinobis(3-ethyl-benzolthiazoline-6-sulphonic acid) by hydrogen peroxide (Cayman Chemical Co.). Various concentrations (0-100 ug/mL) of heparin and rHSs are tested for their ability to block oxidation as monitored by the initial velocity of the reaction at A750. The antioxidant Trolox (Cayman Chemical Co.) is used as a control.

Anti-oxidative capacity is also assessed in cell culture using reduced glutathione (rGSH) as a marker for oxidative stress. A549 (alveolar epithelial cells) are incubated with 0.1 mM H2O2±100 ug/mL of rHS. To determine total GSH (tGSH), cells are lysed and supernatants are incubated with 5,5′-dithiobis-(2-nitrobenzoic acid) (DTNB) and glutathione reductase (GR) for 30 sec at room temperature. Then, β-nicotinamide adenine dinucleotide phosphate (βNADPH) is added and A412 is monitored. Oxidized glutathione (GSSG) is measured by preincubating cell lysates with 2-vinyl pyridine for 1 hour followed by neutralization with triethanolamine before performing the DTNB reaction. rGSH is calculated by subtracting GSSG from tGSH and is normalized to the number of cells.

Inflammatory mediators. The ability of rHSs to compete chemokine binding away from immobilized heparin is tested as described above. 50 ng of pharmaceutical heparin is immobilized in each well of a 96—well microtiter plate using 90% saturated ammonium sulfate. Ten fold dilutions from 200 ug/mL of each rHS is pre-incubated with 10 nM CXCL1 or IL8 as described above. For CXCL5 and CCL2 (R&D Systems), the chemokine is titered in a pilot experiment to determine a working concentration with a robust signal and low background. The chemokine/soluble HS is incubated in each well for 1 hour at room temperature to reach equilibrium. The protein bound to the immobilized heparin is detected using biotinylated primary antibodies (R&D Systems) and streptavidin HRP (Jackson ImmunoResearch). Curves are fit to the data and IC50 values will be calculated (Prism, GraphPad).

The biochemical analysis is followed up with functional assays using primary human neutrophils. Neutrophils are isolated from whole human blood using a Ficoll density gradient. If primary neutrophils are problematic, PLB-985, a human myeloid cell line that can be differentiated to neutrophil-like cells that express chemokine receptors CXCR1 and CXCR2 and have been used for functional assays are used. Neutrophils generate ROS in response to soluble agonists including IL8. Chemokine-mediated oxidative burst and the effect of rHS inhibitors is easily measured in neutrophils using dihydrorhodamine 123 (DHR), which is oxidized in the cytosol to fluorescent rhodamine. Neutrophils are incubated with individual chemokines at various concentrations (0-100 ng/mL) and the oxidative burst response is determined by addition of 150 μM DHR. Subsequently, the inhibitory effect of 0-100 μg/mL rHSs will be tested. The extent of rhodamine formation in the cell is measured by flow cytometry (Guava, Luminex).

Anticoagulation and heparin induced thrombocytopenia. In order for rHS to be widely accepted for clinical use in pulmonary inflammation, the absence of anticoagulant activity must be demonstrated. Artificial substrates for Factor Xa (S2765, Chromogenix) or Thrombin (S2235, Chromogenix) are incubated with antithrombin, which inactivates the enzyme in the presence of anticoagulant heparin/rHS. rHS devoid of 3-O-sulfate has no inhibitory activity in these assays. Using these assays, the selected anti-inflammatory candidates are verified to have no anticoagulant activity. The ability of selected candidates to bind CXCL4 is determined using the competition assay described above. An acceptable candidate has platelet factor 4 binding no greater than that of pharmaceutical heparin. An ideal candidate will have lower binding.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed. It is intended that the following claims define the scope of the invention and that methods and structures within the scope of these claims and their equivalents be covered thereby.

What is claimed is:

1. A method of producing a heparin or a heparan sulfate, the method comprising culturing a genetically modified cell line comprising at least one of a mastocytoma cell line and a basophil neoplastic cell line; and isolating the heparin or heparan sulfate from the cell line.

2. The method of claim 1, wherein the mastocytoma cell line is selected from the group consisting of MST cells, P815 cells, MC/9 cells, SI/SI4 cells, 10P2 cells, 11P0-1 cells, and 10P12 cells.

3. The method of claim 1, wherein the genetically modified cell line is deficient for one or more of chondroitin sulfate synthase 1 (CHSY1), chondroitin sulfate N-acetylgalactosaminyltransferase 2 (CSGALNACT2), chondroitin sulfate N-acetylgalactosaminyltransferase 1 (CSGALNACT1), Heparan sulfate 2-O-sulfotransferase (HS2ST), and heparan sulfate C5-epimerase (GLCE).

4. The method of claim 1, wherein the genetically modified cell line is deficient for a heparan sulfate catabolic enzyme.

5. The method of claim 4, wherein the heparan sulfate catabolic enzyme comprises one or more of heparanase (HPSE), beta-glucoronidase (GUSB), sulfamidase (SGSH), heparan alpha-glucosaminide N-acetyltransferase (HGNAT), alpha-N-acetyl glucosaminidase (GSNAT), uronate-2-O-sulfatases (IDS and GDS), alpha-L-iduronidase (IDUA), and heparan sulfate 6-O-endosulfatase (SULF1-2).

6. The method of claim 1, wherein the genetically modified cell line overexpresses one or more of heparanase, a protease, a polymerase, a sulfotransferase, an endosulfatase, and a proteoglycan protein core.

7. The method of claim 6, wherein the protease comprises one or more of matrix metalloproteinase (MMP), MMP2, MMP3, MMP7, MMP9, MT1-MMP, MT3-MMP, ADAM17, ADAMTS1, ADAMTS4, trypsin, and chymotrypsin.

8. The method of claim 6, wherein the proteoglycan protein core comprises one or more of a serglycin, a syndecan, a glypican, perlecan, and CD44 core protein (CD44E).

9. The method of claim 8, wherein the serglycin comprises a human serglycin, a pig serglycin, a mouse serglycin.

10. The method of claim 8 or claim 9, wherein the number of GAG attachment sites in the serglycin is modified.

11. The method of claim 8, wherein the syndecan is selected from the group consisting of syndecan-1, syndecan-2, syndecan-3, syndecan-4, and syndecan ectodomains.

12. The method of claim 8, wherein the glypican is selected from the group consisting of glypican 1, glypican 2, glypican 3, glypican 4, glypican 5, and glypican 6 and ectodomains.

13. The method of claim 1, wherein the genetically modified cell line overexpresses one or more of Hs3st1, Hs6st1, Hs6st2, Ndst2, or Sulf2.

14. The method of claim 1, wherein the genetically modified cell line overexpresses Hs3st1.

15. The method of claim 1, wherein the genetically modified cell line overexpresses Hs3st1 and Hs6st1.

16. The method of claim 1, wherein the genetically modified cell line overexpresses Hs3st1, Hs6st1, and Sulf2.

17. The method of claim 1, wherein the method comprises degranulating the cell line.

18. The method of claim 17, wherein degranulating the cell line comprises a method selected from the group consisting of Antigen -IgE induced FcεRI aggregation on the degranulating cell surface, contacting the cell line to a degranulating agent, altering the culture temperature, altering the culture medium pH, altering the culture medium salt concentration, and agitation.

19. The method of claim 18, wherein the degranulating agent is selected from the group consisting of calcium ionophore A23187, compound 48/80, tetradecanoyl phorbol acetate (TPA), and substance P.

20. The method of claim 1, wherein the genetically modified cell line is cultured in CDM4NSO medium.

21. The method of claim 1, wherein the genetically modified cell line is cultured in a medium supplemented with xylosides.

22. The method of claim 1, wherein the genetically modified cell line is cultured in suspension culture or in a hollow fiber bioreactor.

23. A high throughput method of quantifying heparan sulfate in a group of samples, the method comprising:
(a) binding each sample to a well of a multi-well chromatography column;
(b) digesting the samples bound to the column with an enzyme;
(c) eluting the samples from the column with a solution comprising a salt; and
(d) measuring the heparan sulfate in the sample using liquid chromatography.

24. The method of claim 23, wherein the chromatography column is selected from at least one of an ion exchange column and a size exclusion column.

25. The method of claim 23, wherein the enzyme is selected from at least one of a nuclease and a protease.

26. The method of claim 23, wherein the salt is a volatile salt.

27. The method of claim 23, wherein the liquid chromatography is an ultra performance liquid chromatography.

28. The method of claim 23, wherein the method comprises liquid chromatography with fluorescently tagged heparan sulfate disaccharides.

29. A high throughput method of quantifying heparan sulfate in a group of samples, the method comprising:
(a) contacting each sample to a well in a multi-well plate, wherein each well is coated with a guanidinylated antibiotic, thereby binding the heparan sulfate in the sample to the plate;
(b) contacting the bound heparan sulfate to a heparan sulfate binding protein;
(c) contacting the bound heparan sulfate binding protein to a detection reagent; and
(d) measuring a signal from the detection reagent,
wherein the signal from the detection reagent corresponds to the amount of heparan sulfate in the sample.

30. The method of claim 29, wherein the guanidinylated antibiotic comprises guanidinylated neomycin.

31. The method of claim 29, wherein the heparan sulfate binding protein is selected from at least one of FGF-2, PF4, ATIII, and VEGF.

32. The method of claim 29, wherein the signal is selected from at least one of a fluorescent signal; a luminescent signal; and a colorimetric signal.

33. The method of claim 29, wherein the signal is generated enzymatically.

34. A method of culturing a stem cell, the method comprising contacting a stem cell to a composition comprising a heparin or a heparan sulfate isolated from a genetically modified cell line.

35. The method of claim 34, wherein the genetically modified cell line comprises at least one of a mastocytoma (MST) cell line and a basophil neoplastic cell line.

36. The method of claim 34, wherein the genetically modified cell line is deficient for one or more of chondroitin sulfate synthase 1 (CHSY1), chondroitin sulfate N-acetylgalactosaminyltransferase 2 (CSGALNACT2), chondroitin sulfate N-acetylgalactosaminyltransferase 1 (CSGALNACT1), Heparan sulfate 2-O-sulfotransferase (HS2ST), and heparan sulfate C5-epimerase (GLCE).

37. The method of claim 34, wherein the genetically modified cell line is deficient for a heparan sulfate catabolic enzyme.

38. The method of claim 37, wherein the heparan sulfate catabolic enzyme comprises one or more of heparanase (HPSE), beta-glucoronidase (GUSB), sulfamidase (SGSH), heparan alpha-glucosaminide N-acetyltransferase (HGNAT), alpha-N-acetyl glucosaminidase (GSNAT), uronate-2-O-sulfatases (IDS), alpha-L-iduronidase (IDUA), and heparan sulfate 6-O-endosulfatase (SULF1-2).

39. The method of claim 34, wherein the genetically modified cell line overexpresses one or more of heparanase, a protease, a polymerase, a sulfotransferase, an endosulfatase, and a proteoglycan protein core.

40. The method of claim 39, wherein the protease comprises one or more of matrix metalloproteinase (MMP), MMP2, MMP3, MMP7, MMP9, MT1-MMP, MT3-MMP, ADAM17, ADAMTS1, ADAMTS4, trypsin, and chymotrypsin.

41. The method of claim 39, wherein the proteoglycan protein core comprises one or more of a serglycin, a syndecan, a glypican, perlecan, and CD44 core protein (CD44E).

42. The method of claim 41, wherein the serglycin comprises a human serglycin, a pig serglycin, a mouse serglycin.

43. The method of claim 41 or claim 42, wherein the number of GAG attachment sites in the serglycin is modified.

44. The method of claim 41, wherein the syndecan is selected from the group consisting of syndecan-1, syndecan-2, syndecan-3, syndecan-4, and syndecan ectodomains.

45. The method of claim 41, wherein the glypican is selected from the group consisting of glypican 1, glypican 2, glypican 3, glypican 4, glypican 5, and glypican 6 and ectodomains.

46. A method of treating a wound in a subject, the method comprising contacting the wound to a composition comprising a heparin or a heparan sulfate isolated from a genetically modified cell line.

47. The method of claim 46, wherein the genetically modified cell line comprises at least one of a mastocytoma (MST) cell line and a basophil neoplastic cell line.

48. The method of claim 46, wherein the wound is selected from at least one of a diabetic wound, an incision, a laceration, an abrasion, an avulsion, a puncture wound, a penetration wound, a gunshot wound, a hematoma, and a crush injury.

49. The method of claim 46, wherein the genetically modified cell line is deficient for one or more of chondroitin sulfate synthase 1 (CHSY1), chondroitin sulfate N-acetylgalactosaminyltransferase 2 (CSGALNACT2), chondroitin sulfate N-acetylgalactosaminyltransferase 1 (CSGALNACT1), Heparan sulfate 2-O-sulfotransferase (HS2ST), and heparan sulfate C5-epimerase (GLCE).

50. The method of claim 46, wherein the genetically modified cell line is deficient for a heparan sulfate catabolic enzyme.

51. The method of claim 50, wherein the heparan sulfate catabolic enzyme comprises one or more of heparanase (HPSE), beta-glucoronidase (GUSB), sulfamidase (SGSH), heparan alpha-glucosaminide N-acetyltransferase (HGNAT), alpha-N-acetyl glucosaminidase (GSNAT), uronate-2-O-sulfatase (IDS and GDS), alpha-L-iduronidase (IDUA), and heparan sulfate 6-O-endosulfatase (SULF1-2).

52. The method of claim 46, wherein the genetically modified MST cell line overexpresses one or more of heparanase, a protease, a polymerase, a sulfotransferase, an endosulfatase, and a proteoglycan protein core.

53. The method of claim 52, wherein the protease comprises one or more of matrix metalloproteinase (MMP), MMP2, MMP3, MMP7, MMP9, MT1-MMP, MT3-MMP, ADAM17, ADAMTS1, ADAMTS4, trypsin, and chymotrypsin.

54. The method of claim 52, wherein the proteoglycan protein core comprises one or more of a serglycin, a syndecan, a glypican, perlecan, and CD44 core protein (CD44E).

55. The method of claim 54, wherein the serglycin comprises a human serglycin, a pig serglycin, a mouse serglycin.

56. The method of claim 54 or claim 55, wherein the number of GAG attachment sites in the serglycin is modified.

57. The method of claim 54, wherein the syndecan is selected from the group consisting of syndecan-1, syndecan-2, syndecan-3, syndecan-4, and syndecan ectodomains.

58. The method of claim 54, wherein the glypican is selected from the group consisting of glypican 1, glypican 2, glypican 3, glypican 4, glypican 5, and glypican 6 and ectodomains.

59. A method of treating a bone fracture in a subject, the method comprising administering an effective amount of a composition comprising a heparin or a heparan sulfate isolated from a genetically modified cell line.

60. The method of claim 59, wherein the genetically modified cell line comprises at least one of a mastocytoma (MST) cell line and a basophil neoplastic cell line.

61. The method of claim 59, wherein the genetically modified cell line is deficient for one or more of chondroitin sulfate synthase 1 (CHSY1), chondroitin sulfate N-acetylgalactosaminyltransferase 2 (CSGALNACT2), chondroitin sulfate N-acetylgalactosaminyltransferase 1 (CSGALNACT1), Heparan sulfate 2-O-sulfotransferase (HS2ST), and heparan sulfate C5-epimerase (GLCE).

62. The method of claim 59, wherein the genetically modified cell line is deficient for a heparan sulfate catabolic enzyme.

63. The method of claim 62, wherein the heparan sulfate catabolic enzyme comprises one or more of heparanase (HPSE), beta-glucoronidase (GUSB), sulfamidase (SGSH), heparan alpha-glucosaminide N-acetyltransferase (HGNAT), alpha-N-acetyl glucosaminidase (GSNAT), uronate-2-O-sulfatase (IDS and GDS), alpha-L-iduronidase (IDUA), and heparan sulfate 6-O-endosulfatase (SULF1-2).

64. The method of claim 59, wherein the genetically modified cell line overexpresses one or more of heparanase, a protease, a polymerase, a sulfotransferase, an endosulfatase, and a proteoglycan protein core.

65. The method of claim 64, wherein the protease comprises one or more of matrix metalloproteinase (MMP), MMP2, MMP3, MMP7, MMP9, MT1-MMP, MT3-MMP, ADAM17, ADAMTS1, ADAMTS4, trypsin, and chymotrypsin.

66. The method of claim 64, wherein the proteoglycan protein core comprises one or more of a serglycin, a syndecan, a glypican, perlecan, and CD44 core protein (CD44E).

67. The method of claim 66, wherein the serglycin comprises a human serglycin, a pig serglycin, a mouse serglycin.

68. The method of claim 66 or claim 67, wherein the number of GAG attachment sites in the serglycin is modified.

69. The method of claim 66, wherein the syndecan is selected from the group consisting of syndecan-1, syndecan-2, syndecan-3, syndecan-4, and syndecan ectodomains.

70. The method of claim 66, wherein the glypican is selected from the group consisting of glypican 1, glypican 2, glypican 3, glypican 4, glypican 5, and glypican 6 and ectodomains.

71. A method of reducing the appearance of a skin condition, the method comprising contacting the skin condition to a composition comprising a heparin or a heparan sulfate isolated from a genetically modified cell line.

72. The method of claim 71, wherein the genetically modified cell line comprises at least one of a mastocytoma (MST) cell line and a basophil neoplastic cell line.

73. The method of claim 71, wherein the skin condition is at least one of a wrinkle, a pimple, a hyperpigmentation, an age-related skin condition, dryness, lack of skin elasticity, lack of skin firmness, and fine lines.

74. The method of claim 71, wherein the genetically modified cell line is deficient for one or more of chondroitin sulfate synthase 1 (CHSY1), chondroitin sulfate N-acetylgalactosaminyltransferase 2 (CSGALNACT2), chondroitin sulfate N-acetylgalactosaminyltransferase 1 (CSGALNACT1), Heparan sulfate 2-O-sulfotransferase (HS2ST), and heparan sulfate C5-epimerase (GLCE).

75. The method of claim 71, wherein the genetically modified cell line is deficient for a heparan sulfate catabolic enzyme.

76. The method of claim 75, wherein the heparan sulfate catabolic enzyme comprises one or more of heparanase (HPSE), beta-glucoronidase (GUSB), sulfamidase (SGSH), heparan alpha-glucosaminide N-acetyltransferase (HGNAT), alpha-N-acetyl glucosaminidase (GSNAT), uronate-2-O-sulfatases (IDS and GDS), alpha-L-iduronidase (IDUA), and heparan sulfate 6-O-endosulfatase (SULF1-2).

77. The method of claim 71, wherein the genetically modified cell line overexpresses one or more of heparanase, a protease, a polymerase, a sulfotransferase, an endosulfatase, and a proteoglycan protein core.

78. The method of claim 77, wherein the protease comprises one or more of matrix metalloproteinase (MMP), MMP2, MMP3, MMP7, MMP9, MT1-MMP, MT3-MMP, ADAM17, ADAMTS1, ADAMTS4, trypsin, and chymotrypsin.

79. The method of claim 77, wherein the proteoglycan protein core comprises one or more of a serglycin, a syndecan, a glypican, perlecan, and CD44 core protein (CD44E).

80. The method of claim 79, wherein the serglycin comprises a human serglycin, a pig serglycin, a mouse serglycin.

81. The method of claim 79 or claim 80, wherein the number of GAG attachment sites in the serglycin is modified.

82. The method of claim 79, wherein the syndecan is selected from the group consisting of syndecan-1, syndecan-2, syndecan-3, syndecan-4, and syndecan ectodomains.

83. The method of claim 79, wherein the glypican is selected from the group consisting of glypican 1, glypican 2, glypican 3, glypican 4, glypican 5, and glypican 6 and ectodomains.

84. A method of treating a cancer in a subject, the method comprising to the subject an effective amount of a composition comprising a heparin or a heparan sulfate isolated from a genetically modified cell line.

85. The method of claim 84, wherein the genetically modified cell line comprises at least one of a mastocytoma (MST) cell line and a basophil neoplastic cell line.

86. The method of claim 84, wherein the cancer is selected from the group consisting of a skin cancer, a lung cancer, a breast cancer, a prostate cancer, a colorectal cancer, a bladder cancer, a melanoma, a lymphoma, a kidney cancer, and a leukemia.

87. The method of claim 84, wherein the genetically modified cell line is deficient for one or more of chondroitin sulfate synthase 1 (CHSY1), chondroitin sulfate N-acetylgalactosaminyltransferase 2 (CSGALNACT2), chondroitin sulfate N-acetylgalactosaminyltransferase 1 (CSGALNACT1), Heparan sulfate 2-O-sulfotransferase (HS2ST), and heparan sulfate C5-epimerase (GLCE).

88. The method of claim 84, wherein the genetically modified cell line is deficient for a heparan sulfate catabolic enzyme.

89. The method of claim 88, wherein the heparan sulfate catabolic enzyme comprises one or more of heparanase (HPSE), beta-glucoronidase (GUSB), sulfamidase (SGSH), heparan alpha-glucosaminide N-acetyltransferase (HGNAT), alpha-N-acetyl glucosaminidase (GSNAT), uronate-2-O-sulfatases (IDS and GDS), alpha-L-iduronidase (IDUA), and heparan sulfate 6-O-endosulfatase (SULF1-2).

90. The method of claim 84, wherein the genetically modified cell line overexpresses one or more of heparanase, a protease, a polymerase, a sulfotransferase, an endosulfatase, and a proteoglycan protein core.

91. The method of claim 90, wherein the protease comprises one or more of matrix metalloproteinase (MMP), MMP2, MMP3, MMP7, MMP9, MT1-MMP, MT3-MMP, ADAM17, ADAMTS1, ADAMTS4, trypsin, and chymotrypsin.

92. The method of claim 90, wherein the proteoglycan protein core comprises one or more of a serglycin, a syndecan, a glypican, perlecan, and CD44 core protein (CD44E).

93. The method of claim 92, wherein the serglycin comprises a human serglycin, a pig serglycin, a mouse serglycin.

94. The method of claim 92 or claim 93, wherein the number of GAG attachment sites in the serglycin is modified.

95. The method of claim 92, wherein the syndecan is selected from the group consisting of syndecan-1, syndecan-2, syndecan-3, syndecan-4, and syndecan ectodomains.

96. The method of claim 92, wherein the glypican is selected from the group consisting of glypican 1, glypican 2, glypican 3, glypican 4, glypican 5, and glypican 6 and ectodomains.

97. A method of modulating angiogenesis in a subject, the method comprising to the subject an effective amount of a composition comprising a heparin or a heparan sulfate isolated from a genetically modified cell line.

98. The method of claim 97, wherein the genetically modified cell line comprises at least one of a mastocytoma (MST) cell line and a basophil neoplastic cell line.

99. The method of claim 97, wherein the method increases angiogenesis in the subject.

100. The method of claim 97, wherein the method decreases angiogenesis in the subject.

101. The method of claim 97, wherein the genetically modified cell line is deficient for one or more of chondroitin sulfate synthase 1 (CHSY1), chondroitin sulfate N-acetylgalactosaminyltransferase 2 (CSGALNACT2), chondroitin sulfate N-acetylgalactosaminyltransferase 1 (CSGALNACT1), Heparan sulfate 2-O-sulfotransferase (HS2ST), and heparan sulfate C5-epimerase (GLCE).

102. The method of claim 97, wherein the genetically modified cell line is deficient for a heparan sulfate catabolic enzyme.

103. The method of claim 102, wherein the heparan sulfate catabolic enzyme comprises one or more of heparanase (HPSE), beta-glucoronidase (GUSB), sulfamidase (SGSH), heparan alpha-glucosaminide N-acetyltransferase (HGNAT), alpha-N-acetyl glucosaminidase (GSNAT), uronate-2-O-sulfatases (IDS and GDS), alpha-L-iduronidase (IDUA), and heparan sulfate 6-O-endosulfatase (SULF1-2).

104. The method of claim 97, wherein the genetically modified cell line overexpresses one or more of heparanase, a protease, a polymerase, a sulfotransferase, an endosulfatase, and a proteoglycan protein core.

105. The method of claim 104, wherein the protease comprises one or more of matrix metalloproteinase (MMP), MMP2, MMP3, MMP7, MMP9, MT1-MMP, MT3-MMP, ADAM17, ADAMTS1, ADAMTS4, trypsin, and chymotrypsin.

106. The method of claim 104, wherein the proteoglycan protein core comprises one or more of a serglycin, a syndecan, a glypican, perlecan, and CD44 core protein (CD44E).

107. The method of claim 106, wherein the serglycin comprises a human serglycin, a pig serglycin, a mouse serglycin.

108. The method of claim 106 or claim 107, wherein the number of GAG attachment sites in the serglycin is modified.

109. The method of claim 106, wherein the syndecan is selected from the group consisting of syndecan-1, syndecan-2, syndecan-3, syndecan-4, and syndecan ectodomains.

110. The method of claim 106, wherein the glypican is selected from the group consisting of glypican 1, glypican 2, glypican 3, glypican 4, glypican 5, and glypican 6 and ectodomains.

111. A method of treating an inflammatory disease in a subject, the method comprising to the subject an effective amount of a composition comprising a heparin or a heparan sulfate isolated from a genetically modified cell line.

112. The method of claim 111, wherein the genetically modified cell line comprises at least one of a mastocytoma (MST) cell line and a basophil neoplastic cell line.

113. The method of claim 111, wherein the inflammatory disease is selected from at least one of the group consisting of chronic obstructive pulmonary disease (COPD), acute respiratory distress syndrome (ARDS), cystic fibrosis, alpha-1 antitrypsin deficiency, a diabetes, an arthritis, psoriasis, multiple sclerosis, systemic lupus erythematosus, an inflammatory bowel disease, Graves' disease, Addison's disease, Sjögren's syndrome, Hashimoto's thyroiditis, Myasthenia gravis, vasculitis, an anemia, and celiac disease.

114. The method of claim 111, wherein the genetically modified cell line is deficient for one or more of chondroitin sulfate synthase 1 (CHSY1), chondroitin sulfate N-acetylgalactosaminyltransferase 2 (CSGALNACT2), chondroitin sulfate N-acetylgalactosaminyltransferase 1 (CSGALNACT1), Heparan sulfate 2-O-sulfotransferase (HS2ST), and heparan sulfate C5-epimerase (GLCE).

115. The method of claim 111, wherein the genetically modified cell line is deficient for a heparan sulfate catabolic enzyme.

116. The method of claim 115, wherein the heparan sulfate catabolic enzyme comprises one or more of heparanase (HPSE), beta-glucoronidase (GUSB), sulfamidase (SGSH), heparan alpha-glucosaminide N-acetyltransferase (HGNAT), alpha-N-acetyl glucosaminidase (GSNAT), uronate-2-O-sulfatases (IDS and GDS), alpha-L-iduronidase (IDUA), and heparan sulfate 6-O-endosulfatase (SULF1-2).

117. The method of claim 111, wherein the genetically modified cell line overexpresses one or more of heparanase, a protease, a polymerase, a sulfotransferase, an endosulfatase, and a proteoglycan protein core.

118. The method of claim 117, wherein the protease comprises one or more of matrix metalloproteinase (MMP), MMP2, MMP3, MMP7, MMP9, MT1-MMP, MT3-MMP, ADAM17, ADAMTS1, ADAMTS4, trypsin, and chymotrypsin.

119. The method of claim 117, wherein the proteoglycan protein core comprises one or more of a serglycin, a syndecan, a glypican, perlecan, and CD44 core protein (CD44E).

120. The method of claim 119, wherein the serglycin comprises a human serglycin, a pig serglycin, a mouse serglycin.

121. The method of claim 119 or claim 120, wherein the number of GAG attachment sites in the serglycin is modified.

122. The method of claim 119, wherein the syndecan is selected from the group consisting of syndecan-1, syndecan-2, syndecan-3, syndecan-4, and syndecan ectodomains.

123. The method of claim 119, wherein the glypican is selected from the group consisting of glypican 1, glypican 2, glypican 3, glypican 4, glypican 5, and glypican 6 and ectodomains.

124. A method of treating a neurological disease or condition in a subject, the method comprising to the subject an effective amount of a composition comprising a heparin or a heparan sulfate isolated from a genetically modified cell line.

125. The method of claim 124, wherein the genetically modified cell line comprises at least one of a mastocytoma (MST) cell line and a basophil neoplastic cell line.

126. The method of claim 124, wherein the neurological disease or condition is selected from at least one of the group consisting of Alzheimer's disease, Parkinson's disease, Huntington's disease, Amyotrophic lateral sclerosis, Dementia, Transmissible spongiform encephalopathy, Dentatorubro-pallidoluysian atrophy, Spinal and bulbar muscular atrophy, Spinocerebellar ataxia Type 1, Spinocerebellar ataxia Type 2, Spinocerebellar ataxia Type 3, Spinocerebellar ataxia Type 6, Spinocerebellar ataxia Type 7, and Spinocerebellar ataxia Type 17.

127. The method of claim 124, wherein the genetically modified cell line is deficient for one or more of chondroitin sulfate synthase 1 (CHSY1), chondroitin sulfate N-acetylgalactosaminyltransferase 2 (CSGALNACT2), chondroitin sulfate N-acetylgalactosaminyltransferase 1 (CSGALNACT1), Heparan sulfate 2-O-sulfotransferase (HS2ST), and heparan sulfate C5-epimerase (GLCE).

128. The method of claim 124, wherein the genetically modified cell line is deficient for a heparan sulfate catabolic enzyme.

129. The method of claim 128, wherein the heparan sulfate catabolic enzyme comprises one or more of heparanase (HPSE), beta-glucoronidase (GUSB), sulfamidase (SGSH), heparan alpha-glucosaminide N-acetyltransferase (HGNAT), alpha-N-acetyl glucosaminidase (GSNAT), uronate-2-O-sulfatases (IDS and GDS), alpha-L-iduronidase (IDUA), and heparan sulfate 6-O-endosulfatase (SULF1-2).

130. The method of claim 124, wherein the genetically modified cell line overexpresses one or more of heparanase, a protease, a polymerase, a sulfotransferase, an endosulfatase, and a proteoglycan protein core.

131. The method of claim 130, wherein the protease comprises one or more of matrix metalloproteinase (MMP), MMP2, MMP3, MMP7, MMP9, MT1-MMP, MT3-MMP, ADAM17, ADAMTS1, ADAMTS4, trypsin, and chymotrypsin.

132. The method of claim 130, wherein the proteoglycan protein core comprises one or more of a serglycin, a syndecan, a glypican, perlecan, and CD44 core protein (CD44E).

133. The method of claim 132, wherein the serglycin comprises a human serglycin, a pig serglycin, a mouse serglycin.

134. The method of claim 132 or claim 133, wherein the number of GAG attachment sites in the serglycin is modified.

135. The method of claim 132, wherein the syndecan is selected from the group consisting of syndecan-1, syndecan-2, syndecan-3, syndecan-4, and syndecan ectodomains.

136. The method of claim 132, wherein the glypican is selected from the group consisting of glypican 1, glypican 2, glypican 3, glypican 4, glypican 5, and glypican 6 and ectodomains.

137. A composition comprising a heparin or a heparan sulfate, wherein the heparin or heparan sulfate has reduced 2-0-sulfation and wherein the heparin or heparan sulfate has reduced affinity for platelet factor 4 (PF4) than an unfractionated heparin preparation.

138. The composition of claim 137, wherein the composition is purified from a mastocytoma (MST) cell line or a basophil neoplastic cell line genetically modified to be deficient for Heparan sulfate 2-O-sulfotransferase (HS2ST).

139. The composition of claim 137 or claim 138, wherein the composition is purified from a MST cell line or a basophil neoplastic cell line genetically modified to overexpress Heparan sulfate-6-O-endosulfatase 2 (Sulf-2).

140. A method of reducing blood clots in a subject, the method comprising administering a composition comprising a heparin or a heparan sulfate having reduced affinity for platelet factor 4 compared to an unfractionated heparin preparation.

141. The method of claim 140, wherein the composition is purified from a mastocytoma (MST) cell line or a basophil neoplastic cell line genetically modified to be deficient for Heparan sulfate 2-O-sulfotransferase (HS2ST).

142. The method of claim 140 or claim 141, wherein the composition is purified from a MST cell line or a basophil neoplastic cell line genetically modified to overexpress Heparan sulfate-6-O-endosulfatase 2 (Sulf-2).

143. The method of claim 140, wherein the composition is purified from a MST cell line or a basophil neoplastic cell line genetically modified to be deficient for one or more of chondroitin sulfate synthase 1 (CHSY1), chondroitin sulfate N-acetylgalactosaminyltransferase 2 (CSGALNACT2), chondroitin sulfate N-acetylgalactosaminyltransferase 1 (CSGALNACT1), Heparan sulfate 2-O-sulfotransferase (HS2ST), and heparan sulfate C5-epimerase (GLCE).

144. The method of claim 140, wherein the composition is purified from a MST cell line or a basophil neoplastic cell line genetically modified to be deficient for a heparan sulfate catabolic enzyme.

145. The method of claim 144, wherein the heparan sulfate catabolic enzyme comprises one or more of heparanase (HPSE), beta-glucoronidase (GUSB), sulfamidase (SGSH), heparan alpha-glucosaminide N-acetyltransferase (HGNAT), alpha-N-acetyl glucosaminidase (GSNAT), uronate-2-O-sulfatases (IDS and GDS), alpha-L-iduronidase (IDUA), and heparan sulfate 6-O-endosulfatase (SULF1-2).

146. The method of claim 140, wherein the composition is purified from a MST cell line or a basophil neoplastic cell line genetically modified to overexpress one or more of heparanase, a protease, a polymerase, a sulfotransferase, an endosulfatase, and a proteoglycan protein core.

147. The method of claim 146, wherein the protease comprises one or more of matrix metalloproteinase (MMP), MMP2, MMP3, MMP7, MMP9, MT1-MMP, MT3-MMP, ADAM17, ADAMTS1, ADAMTS4, trypsin, and chymotrypsin.

148. The method of claim 146, wherein the proteoglycan protein core comprises one or more of a serglycin, a syndecan, a glypican, perlecan, and CD44 core protein (CD44E).

149. The method of claim 148, wherein the serglycin comprises a human serglycin, a pig serglycin, a mouse serglycin.

150. The method of claim 148 or claim 149, wherein the number of GAG attachment sites in the serglycin is modified.

151. The method of claim 148, wherein the syndecan is selected from the group consisting of syndecan-1, syndecan-2, syndecan-3, syndecan-4, and syndecan ectodomains.

152. The method of claim 148, wherein the glypican is selected from the group consisting of glypican 1, glypican 2, glypican 3, glypican 4, glypican 5, and glypican 6 and ectodomains.

153. A composition comprising a heparin or a heparan sulfate, wherein the heparin or heparan sulfate has increased 2-0-sulfate compared to pharmaceutical heparin.

154. The composition of claim 153, wherein the heparin or the heparan sulfate has improved anti-inflammatory activity compared with pharmaceutical heparin.

155. The composition of claim 153, wherein the composition is purified from a mastocytoma (MST) cell line or a basophil neoplastic cell line genetically modified to be overexpress Heparan sulfate 2-O-sulfotransferase (HS2ST).

156. A method of treating a lung inflammatory disease in a subject, the method comprising to the subject an effective amount of a composition comprising a heparin or a heparan sulfate isolated from a genetically modified cell line.

157. The method of claim 156, wherein the genetically modified cell line comprises at least one of a mastocytoma (MST) cell line and a basophil neoplastic cell line.

158. The method of claim 156, wherein the inflammatory disease is selected from at least one of the group consisting of chronic obstructive pulmonary disease (COPD), acute respiratory distress syndrome (ARDS), cystic fibrosis, alpha-1 antitrypsin deficiency, and an asthma.

159. The method of claim 156, wherein the genetically modified cell line is deficient for one or more of chondroitin sulfate synthase 1 (CHSY1), chondroitin sulfate N-acetylgalactosaminyltransferase 2 (CSGALNACT2), chondroitin sulfate N-acetylgalactosaminyltransferase 1 (CSGALNACT1), and heparan sulfate C5-epimerase (GLCE).

160. The method of claim 156, wherein the genetically modified cell line is deficient for a heparan sulfate catabolic enzyme.

161. The method of claim 160, wherein the heparan sulfate catabolic enzyme comprises one or more of heparanase (HPSE), beta-glucoronidase (GUSB), sulfamidase (SGSH), heparan alpha-glucosaminide N-acetyltransferase (HGNAT), alpha-N-acetyl glucosaminidase (GSNAT), uronate-2-O-sulfatases (IDS and GDS), alpha-L-iduronidase (IDUA), and heparan sulfate 6-O-endosulfatase (SULF1-2).

162. The method of claim 156, wherein the genetically modified cell line overexpresses one or more of heparanase, a protease, a polymerase, a sulfotransferase, an endosulfatase, and a proteoglycan protein core.

163. The method of claim 162, wherein the sulfotransferase is Heparan Sulfate 2-O-Sulfotransferase 1.
